(19) United States
(12) Patent Application Publication
(10) Pub. No.: US 2002/0007053 A1
(43) Pub. Date: Jan. 17, 2002

(54) Glyphosate-tolerant 5-enolpyruvylshikimate-3-phosphate synthases

(76) Inventors: Gerard Francis Barry, St. Louis, MO (US); Ganesh Murthy Kishore, Chesterfield, MO (US); Stephen Rogers Padgette, Grover, MO (US); William Carlton Stallings, Glencoe, MO (US)

Correspondence Address: Janelle D. Waack, HOWREY SIMON ARNOLD & WHITE, LLP, 750 Bering Drive, Houston, TX, 77057-2198, US

(21) Appl. No.: 09/861,696

(22) Filed: May 21, 2001

Related U.S. Application Data
(63) Continuation of application No. 09/137,440, filed on Aug. 20, 1998, now Pat. No. 6,248,876.
Application No. 09/137,440 is a continuation of application No. 08/833,485, filed on Apr. 7, 1997, now Pat. No. 5,804,425.
Application No. 08/833,485 is a continuation of application No. 07/306,063, filed on Feb. 6, 1989, now Pat. No. 4,940,826.
Application No. 07/306,063 is a continuation-in-part of application No. 07/749,611, filed on Aug. 28, 1991, now abandoned.
Application No. 07/749,611 is a continuation-in-part of application No. 07/576,537, filed on Aug. 31, 1990, now abandoned.

(51) Int. Cl. C07H 21/04

(52) U.S. Cl. 536/23.2

(57) ABSTRACT

Genes encoding Class II EPSPS enzymes are disclosed. The genes are useful in producing transformed bacteria and plants which are tolerant to glyphosate herbicide. Class II EPSPS genes share little homology with known, Class I EPSPS genes, and do not hybridize to probes from Class I EPSPS's. The Class II EPSPS enzymes are characterized by being more kinetically efficient than Class I EPSPS's in the presence of glyphosate. Plants transformed with Class II EPSPS genes are also disclosed as weU as a method for selectively controlling weeds in a planted transgenic crop field.

This is a continuation-in-part of a U.S. patent application Ser. No. 07/749,611, filed Aug. 28, 1991 which is a continuation-in-part of U.S. patent application Ser. No. 07/576,537, filed Aug. 31, 1990, now abandoned.

### BACKGROUND OF THE INVENTION

This invention relates in general to plant molecular biology and, more particularly, to a new class of glyphosate-tolerant 5-enolpyruvylshikimate-3-phosphate synthases.

Recent advances in genetic engineering have provided the requisite tools to transform plants to contain foreign genes. It is now possible to produce plants which have unique characteristics of agronomic importance. Certainly, one such advantageous trait is more cost effective, environmentally compatible weed control via herbicide tolerance. Herbicide-tolerant plants may reduce the need for tillage to control weeds thereby effectively reducing soil erosion.

One herbicide which is the subject of much investigation in this regard is N-phosphonomethylglycine commonly referred to as glyphosate. Glyphosate inhibits the shikimic acid pathway which leads to the biosynthesis of aromatic compounds including amino acids. plant hormones and vitamins. Specifically, glyphosate curbs the conversion of phosphoenolpyruvic acid (PEP) and 3-phosphoshikimic acid to 5-enolpyruvyl-3-phosphoshikimic acid by inhibiting the enzyme 5-enolpyruvylshikimate-3-phosphate synthase (hereinafter referred to as EPSP synthase or EPSPS). For purposes of the present invention, the term “glyphosate” should be considered to include any herbicidally effective form of N-phosphonomethylglycine (including any salt thereof) and other forms which result in the production of the glyphosate anion in planta.

It has been shown that glyphosate-tolerant plants can be produced by inserting into the genome of the plant the capacity to produce a higher level of EPSP synthase in the chloroplast of the cell (Shah et al., 1986) which enzyme is preferably glyphosate-tolerant (Kishore et al. 1988). Variants of the wild-type EPSPS enzyme have been isolated which are glyphosate-tolerant as a result of alterations in the EPSPS amino acid coding sequence (Kishore and Shah, 1988; Schulz et al., 1984; Sost et al., 1984; Kishore et al., 1986). These variants typically have a higher Kᵢ for glyphosate than the wild-type EPSPS enzyme which confers the glyphosate-tolerant phenotype, but these variants are also characterized by a high Kₘ for PEP which makes the enzyme kinetically less efficient (Kishore and Shah, 1988; Sost et al., 1984; Schulz et al., 1984; Kishore et al., 1986; Sost and Amrhein, 1990). For example, the apparent Kₘ for PEP and the apparent Ki for glyphosate for the native EPSPS from E. coli are 10 μM and 0.5 μM while for a glyphosate-tolerant isolate having a single amino acid substitution of an alanine for the glycine at position 96 these values are 220 μM and 4.0 mM, respectively. A number of glyphosate-tolerant plant variant EPSPS genes have been constructed by mutagenesis. Again, the glyphosate-tolerant EPSPS was impaired due to an increase in the Kₘ for PEP and a slight reduction of the Vₘₐₓ of the native plant enzyme (Kishore and Shah, 1988) thereby lowering the catalytic efficiency (Vₘₐₓ/Kₘ) of the enzyme. Since the kinetic constants of the variant enzymes are impaired with respect to PEP, it has been proposed that high levels of overproduction of the variant enzyme, 40-80 fold, would be required to maintain normal catalytic activity in plants in the presence of glyphosate (Kishore et al., 1988).

While such variant EPSP synthases have proved useful in obtaining transgenic plants tolerant to glyphosate, it would be increasingly beneficial to obtain an EPSP synthase that is highly glyphosate-tolerant while still kinetically efficient such that the amount of the glyphosate-tolerant EPSPS needed to be produced to maintain normal catalytic activity in the plant is reduced or that improved tolerance be obtained with the same expression level.

Previous studies have shown that EPSPS enzymes from different sources vary widely with respect to their degree of sensitivity to inhibition by glyphosate. A study of plant and bacterial EPSPS enzyme activity as a function of glyphosate concentration showed that there was a very wide range in the degree of sensitivity to glyphosate. The degree of sensitivity showed no correlation with any genus or species tested (Schulz et al., 1985). Insensitivity to glyphosate inhibition of the activity of the EPSPS from the Pseudomonas sp. PG2982 has also been reported but with no details of the studies (Fitzgibbon, 1988). In general, while such natural tolerance has been reported, there is no report suggesting the kinetic superiority of the naturally occurring bacterial glyphosate-tolerant EPSPS enzymes over those of mutated EPSPS enzymes nor have any of the genes been characterized. Similarly, there are no reports on the expression of naturally glyphosate-tolerant EPSPS enzymes in plants to confer glyphosate tolerance.

For purposes of the present invention the term “mature EPSP synthase” relates to the EPSPS polypeptide without the N-terminal chloroplast transit peptide. It is now known that the precursor form of the EPSP synthase in plants (with the transit peptide) is expressed and upon delivery to the chloroplast, the transit peptide is cleaved yielding the mature EPSP synthase. Al numbering of amino acid positions are given with respect to the mature EPSP synthase (without chloroplast transit peptide leader) to facilitate comparison of EPSPS sequences from sources which have chloroplast transit peptides (i.e., plants and fungi) to sources which do not utilize a chloroplast targeting signal (i.e., bacteria).

In the amino acid sequences which follow, the standard single letter or three letter nomenclature are used. All peptide structures represented in the following description are shown in conventional format in which the amino group at the N-terminus appears to the left and the carboxyl group at the C-terminus at the right. Likewise, amino acid nomenclature for the naturally occurring amino acids found in protein is as follows: alanine (Ala;A), asparagine (Asn;N), aspartic acid (Asp;D), arginine (Arg;R), cysteine (Cys;C), glutamic acid (Glu;E), glutamine (Gln;Q), glycine (Gly;G), histidine (His;H), isoleucine (Ile;I), leucine (Leu;L), lysine (Lys;K), methionine (Met;M), phenylalanine (Phe;F), proline (Pro;P), serine (Ser;S), threonine (Thr;T), tryptophan (Trp;W), tyrosine (Tyr;Y), and valine (Val;V). An “X” is used when the amino acid residue is unknown and parentheses designate that an unambiguous assignment is not possible and the amino acid designation within the parentheses is the most probable estimate based on known information.

The term “nonpolar” amino acids include alanine, valine, leucine, isoleucine, proline, phenylalanine, tryptophan, and methionine. The term “uncharged polar” amino acids include glycine, serine. threonine, cysteine, tyrosine, asparagine and glutamine. The term “charged polar” amino acids includes the “acidic” and “basic” amino acids. The term “acidic” amino acids includes aspartic acid and glutamic acid. The term “basic” amino acid includes lysine, arginine and histidine. The term “polar” amino acids includes both “charged polar” and “uncharged polar” amino acids.

Deoxyribonucleic acid (DNA) is a polymer comprising four mononucleotide umts. dAMP (2′-Deoxyadenosine-5-monophosphate), dGMP (2′-Deoxyguanosine-5-monophosphate), dCMP (2′-Deoxycytosine-5-monophosphate) and dTMP (2′-Deoxythymosine-5-monophosphate) linked in various sequences by 3′,5′-phosphodiester bridges. The structural DNA consists of multiple nucleotide triplets called “codons” which code for the amino acids. The codons correspond to the various amino acids as follows: Arg (CGA, CGC, CGG, CGT, AGA, AGG); Leu (CTA, CTC, CTG, CTT, TTA, TTG); Ser (TCA, TCC, TCG, TCT, AGC, AGT); Thr (ACA, ACC, ACG, ACT); Pro (CCA, CCC, CCG, CCT); Ala (GCA, GCC, GCG, GCT); Gly (GGA, GGC, GGG, GGT); Ile (ATA, ATC, ATT); Val (GTA, GTC, GTG, GTT); Lys (AAA, AAG); Asn (AAC, AAT); Gln (AA, CAG); His (CAC, CAT); Glu (GAA, GAG); Asp (GAC, GAT); Tyr (TAC, TAT); Cys (TGC, TGT); Phe (rTC, T=i; Met (ATG); and Trp (UGG). Moreover, due to the redundancy of the genetic code (i.e., more than one codon for all but two amino acids), there are many possible DNA sequences which may code for a particular amino acid sequence.

### SUMMARY OF THE INVENTION

DNA molecules comprising DNA encoding kinetically efficient, glyphosate-tolerant EPSP synthases are disclosed. The EPSP synthases of the present invention reduce the amount of overproduction of the EPSPS enzyme in a transgenic plant necessary for the enzyme to maintain catalytic activity while still conferring glyphosate tolerance. The EPSP synthases described herein represent a new class of EPSPS enzymes, referred to hereinafter as Class II EPSPS enzymes. Class II EPSPS enzymes of the present invention usually share only between about 47% and 55% amino acid similarity or between about 22% and 30% amino acid identity to other known bacterial or plant EPSPS enzymes and exhibit tolerance to glyphosate while maintaining suitable K (PEP) ranges. Suitable ranges of Kₘ (PEP) for EPSPS for enzymes of the present invention are between 1-150 μM, with a more preferred range of between 1-35 μM. and a most preferred range between 2-25 μM. These kinetic constants are determined under the assay conditions specified hereinafter. An EPSPS of the present invention preferably has a Kᵢ for glyphosate range of between 15-10000 μM. The Kᵢ/Kₘ ratio should be between about 2-500, and more preferably between 25-500. The Vₘₐₓ of the purified enzyme should preferably be in the range of 2-100 units/mg (μmoles/minute.mg at 25° C.) and the Kₘ for shikimate-3-phosphate should preferably be in the range of 0.1 to 50 μM.

Genes coding for Class II EPSPS enzymes have been isolated from five (5) different bacteria: Agrobacterium tumefaciens sp. strain CP4, Achromobacter sp. strain LBAA, Pseudomonas sp. strain PG2982, Bacilus subtilis, and Staphylococcus aureus. The LBAA and PG2982 Class II EPSPS genes have been determined to be identical and the proteins encoded by these two genes are very similar to the CP4 protein and share approximately 84% amino acid identity with it. Class II EPSPS enzymes often may be distinguished from Class I EPSPS's by their inability to react with polyclonal antibodies prepared from Class I EPSPS enzymes under conditions where other Class I EPSPS enzymes would readily react with the Class I antibodies as well as the presence of certain unique regions of amino acid homology which are conserved in Class II EPSP synthases as discussed hereinafter.

Other Class II EPSPS enzymes can be readily isolated and identified by utilizing a nucleic acid probe from one of the Class II EPSPS genes disclosed herein using standard hybridization techniques. Such a probe from the CP4 strain has been prepared and utilized to isolate the Class II EPSPS genes from strains LBAA and PG2982. These genes may also optionally be adapted for enhanced expression in plants by known methodology. Such a probe has also been used to identify homologous genes in bacteria isolated de novo from soil.

The Class II EPSPS enzymes are preferably fused to a chloroplast transit peptide (CTP) to target the protein to the chloroplasts of the plant into which it may be introduced. Chimeric genes encoding this CTP-Class II EPSPS fusion protein may be prepared with an appropriate promoter and 3′ polyadenylation site for introduction into a desired plant by standard methods.

To obtain the maximal tolerance to glyphosate herbicide it is preferable to transform the desired plant with a plant-expressible Class II EPSPS gene in conjunction with another plant-expressible gene which expresses a protein capable of degrading glyphosate such as a plant-expressible gene encoding a glyphosate oxidoreductase enzyme as described in PCT Application No. WO 92/00377, the disclosure of which is hereby incorporated by reference.

Therefore, in one aspect, the present invention provides a new class of EPSP synthases that exhibit a low Kₘ for phosphoenolpyruvate (PEP), a high Vₘₐₓ/Kₘ ratio, and a high Kᵢ for glyphosate such that when introduced into a plant, the plant is made glyphosate-tolerant such that the catalytic activity of the enzyme and plant metabolism are maintained in a substantially normal state. For purposes of this discussion, a highly efficient EPSPS refers to its efficiency in the presence of glyphosate.

More particularly, the present invention provides EPSPS enzymes having a Kₘ for phosphoenolpyruvate (PEP) between 1-150 μM and a Kᵢ(glyphosate)/Kₘ(PEP) ratio between 3-500, said enzymes having the sequence domains:

—R—X₁—H—X₂—E— (SEQ ID NO:37), in which

X₁ is an uncharged polar or acidic amino acid,

X₂ is serine or threonine; and

—G—D—K—X₃-(SEQ ID NO:38), in which

X3 is serine or threonine; and

—S—A—Q-X₄—K— (SEQ ID NO:39), in which

X₄ is any amino acid; and

—N—X₅T—R— (SEQ ID:40), in which

X₅ is any amino acid.

Exemplary Class II EPSPS enzyme sequences are disclosed from seven sources: Agrobacterium sp. strain designated CP4, Achromobacter sp. strain LBAA, Pseudomonas sp. strain PG2982, Bacillus subtilis LA2, Staphylococcus aureus (ATCC 35556), Synechocystis sp. PCC6803 and Dichelobacter nodosus.

In another aspect of the present invention, a double-stranded DNA molecule comprising DNA encoding a Class II EPSPS enzyme is disclosed. Exemplary Class II EPSPS enzyme DNA sequences are disclosed from seven sources: Agrobacterium sp. strain designated CP4, Achromobacter sp. strain LBAA, Pseudomonas sp. strain PG2982, Bacillus subtilis 1A2, Staphyloccus aureus (ATCC 35556), Synechocystis sp. PCC6803 and Dichelobacter nodosus.

In a further aspect of the present invention, nucleic acid probes from EPSPS Class II genes are presented that are suitable for use in screening for Class II EPSPS genes in other sources by assaying for the ability of a DNA sequence from the other source to hybridize to the probe.

In yet another aspect of the present invention, a recombinant, double-stranded DNA molecule comprising in sequence:

a) a promoter which functions in plant cells to cause the production of an RNA sequence;

b) a structural DNA sequence that causes the production of an RNA sequence which encodes a Class II EPSPS enzyme having the sequence domains:

—R—X₁—H—X₂—E— (SEQ ID NO:37), in which

X₁ is an uncharged polar or acidic amino acid.

X₂ is serine or threonine; and

—GSD—K—X₃-(SEQ ID NO:38), in which

X₃ is serine or threonine; and

—S—A—Q—X₄-K-(SEQ ID NO:39), in which

X₄ is any amino acid; and

—N—X₅—T—R—(SEQ ID:40), in which

X₅ is any amino acid: and

a 3′ nontranslated region which functions in plant cells to cause the addition of a stretch of polyadenyl nucleotides to the 3′ end of the RNA sequence where the promoter is heterologous with respect to the structural DNA sequence and adapted to cause sufficient expression of the EPSP synthase polypeptide to enhance the glyphosate tolerance of a plant cell transformed with said DNA molecule.

In still yet another aspect of the present invention, transgenic plants and transformed plant cells are disclosed that are made glyphosatetolerant by the introduction of the above-described plant-expressible Class II EPSPS DNA molecule into the plant's genome.

In still another aspect of the present invention, a method for selectively controlling weeds in a crop field is presented by planting crop seeds or crop plants transformed with a plant-expressible Class II EPSPS DNA molecule to confer glyphosate tolerance to the plants which allows for glyphosate containing herbicides to be applied to the crop to selectively kill the glyphosate sensitive weeds, but not the crops.

Other and further objects, advantages and aspects of the invention will become apparent from the accompanying drawing figures and the description of the invention.

### BRIEF DESCRIPTION OF THE DRAWINGS

FIGS. 1A and 1B show the DNA sequence (SEQ ID NO:1) for the full-length promoter of figwort mosaic virus (FDAV35S).

FIG. 2 shows the cosmid cloning vector pMON17020.

FIGS. 3A, 3B, 3C and 3D shows the structural DNA sequence (SEQ ID NO:2) for the Class II EPSPS gene from bacterial isolate Agrobacterium sp. strain CP4 and the deduced aminozcd stqunce (SEQ ID NO:3).

FIGS. 4A-4b shows thle structural DNA sequence (SEQ ID NO:4) for the Class II EPSPS gene from the bacterial isolate Achromobacter sp. strain LBAA and the deduced amino acid sequence (SEQ ID NO:5).

FIGS. 5A-5B show the structural DNA sequence (SEQ ID NO:6) for the Class/II EPSPS gene from the bacterial isolate Pseudomonas sp. strain PG2982 and thd ced uno acid sequence (SEQ ID NO:7).

FIGS. 6A-6B show the Bestfit comparison of the CP4 EPSPS amino acid sequen that for the E. coli EPSPS (SEQ ID NO:8).

FIGS. 7A-7B show the Bestfit comparison of the CP4 EPSPS amino acid sequence (SEQ ID NO:3) with that for the LBAA EPSPS (SEQ ID NO:5).

FIGS. 8A-8B shows the structural DNA sequence (SEQ ID NO:9) for the synthetic CP4 Class II EPSPS gene.

FIG. 9 shows the DNA sequence (SEQ ID NO:10) of the chloroplast transit peptide (CTP) and encoded amino acid sequence (SEQ ID NO:11) derived from the Arabidopsis thaliana EPSPS CTP and containing a SphI restriction site at the chloroplast processing site, hereinafter referred to as CTP2. , o

FIGS. 10A and 10B shows the DNA sequence (SEQ ID NO:12) of the chloroplast transit peptide and encoded amino acid sequence (SEQ ID NO:13) derived from the Arabidopsis thaliana EPSPS gene and containing an EcoRI restriction site within the mature region of the EPSPS, hereinafter referred to as CTP3.

FIG. 11 shows the DNA sequence (SEQ ID NO: 14) of the chloroplast transit peptide and encoded amino acid sequence (SEQ ID NO:15) derived from the Petunia hybrida EPSPS CTP and containing a SphI restriction site at the chloroplast processing site and in which the amino acids at the processing site are change to -Cys-Met hereinafter referred to as CTP4.

FIGS. 12A and 12B show the DNA sequence (SEQ ID NO:16) of the chloroplast transit peptide and encoded amino acid sequence (SEQ ID NO:17) derived from the Petunia hybrida EPSPS gene with the naturally occurring EcoRI site in the mature region of the EPSPS gene. hereinafter referred to as CTP5.

FIG. 13 shows a plasmid map of CP4 plant transformation/expression vector pMON17110.

FIG. 14 shows a plasmid map of CP4 synthetic EPSPS gene plant transformation/expression vector pMON17131.

FIG. 15 shows a plasmid map of CP4 EPSPS free DNA plant transformation expression vector pMON13640.

FIG. 16 shows a plasmid map of CP4 plant transformation/direct selection vector pMON17227.

FIG. 17 shows a plasmid map of CP4 plant transformation/expression vector MON19653.

FIGS. 18A-18D show the structural DNA sequence (SEQ ID NO:41) for the Class II EPSPS gene from the bacterial isolate Bacillus subtilis and the deduced amino acid sequence (SEQ ID NO:42).

FIGS. 19A-19D show the structural DNA sequence (SEQ ID NO:43) for the Class II EPSPS gene from the bacterial isolate Staphylococcus aureus and the deduced amino acid sequence (SEQ ID NO:44).

FIGS. 20A-20 B show the Bestfit comparison of the representative Class II EPSPS amino acid sequences Pseucomonas sp. strain PG2982 (SEQ ID NO:7), Achromobacter sp. strain LBAA (SEQ ID NO:5), Agrobacterium sp. strain designated CP4 (SEQ ID NO:3), Bacillus subtilis (SEQ ID NO:42), and Staphylococcus aureus (SEQ ID NO:44) with that for representative Class I EPSPS amino acid sequences [Sacchromyces cerevisiae (SEQ ID NO:49), Aspergillus nidulans (SEQ ID NO:50), Brassica napus (SEQ ID NO:51), Arabidopsis thaliana (SEQ ID NO:52), Nicotina tobacum (SEQ ID NO:53), L. esculentum (SEQ ID NO:54), Petunia hybrida (SEQ ID NO:55), Zea mays (SEQ ID NO:56), Salmenella gallinarum (SEQ ID NO:57), Salmenella typhimurium (SEQ ID NO:58), Salmenella typhi (SEQ ID NO:65), E. coli (SEQ ID NO:8), K. pneumoniae (SEQ ID NO:59), Y. enterocolitica (SEQ ID NO:60), H. influenzae (SEQ ID NO:61). P. multocida (SEQ ID NO:62), Aeromonas salmonicida (SEQ ID NO:63), Bacillus pertussis (SEQ ID NO:64)] and illustrates the conserved regions among Class II EPSPS sequences which are unique to Class II EPSPS sequences. To aid in a comparison of the EPSPS sequences, only mature EPSPS sequences were compared. That is, the sequence corresponding to the chloroplast transit peptide, if present in a subject EPSPS, was removed prior to m atpnent.

FIGS. 21A-21E show the structural DNA sequence (SEQ ID NO:66) for the Class II EPSPS gene from the bacterial isolate Synechocystis sp. PCC6803 and the deduced amino acsquenf (SEQ ID NO:67).

FIGS. 22A-22E show the structural DNA sequence (SEQ ID NO:68) for the Class II EPSPS gene from the bacterial isolate Dichelobacter nodosus and the deduced acidsequence)(SEQ ID NO:69).

FIGS. 23A-23D show the Bestfit comparison of the representative Class II EPSPS amino acid sequences Pseudomonas sp. strain PG2982 (SEQ ID NO:7), Achromobacter sp. strain LBAA (SEQ ID NO:5), Agrobacterium sp. strain designated CP4 (SEQ ID NO:3), Synechocystis sp. PCC6803 (SEQ ID NO:67), Bacillus subtilis (SEQ ID NO:42), Dichelobacter nodosus (SEQ ID NO:69) and Staphylococcus aureus (SEQ ID NO:44).

FIG. 24 a plasmid map of canola plant transformation/expression vector pMON17209.

FIG. 25 a plasmid map of canola plant transformation/expression vector pMON17237.

### STATEMENT OF THE INVENTION

The expression of a plant gene which exists in double-stranded DNA form involves synthesis of messenger RNA (mRNA) from one strand of the DNA by RNA polymerase enzyme, and the subsequent processing of the MRNA primary transcript inside the nucleus. This processing involves a 3′ non-translated region which adds polyadenylate nucleotides to the 3′ end of the RNA.

Transcription of DNA into mRNA is regulated by a region of DNA usually referred to as the “promoter.” The promoter region contains a sequence of bases that signals RNA polymerase to associate with the DNA, and to initiate the transcription into mRNA using one of the DNA strands as a template to make a corresponding complementary strand of RNA. A number of promoters which are active in plant cells have been described in the literature. These include the nopaline synthase (NOS) and octopine synthase (OCS) promoters. (which are carried on tumor-inducing plasmids of Agrobacterium tumefaciens), the cauliflower mosaic virus (CaMV) 19S and 35S promoters, the light-inducible promoter from the small subunit of ribulose bis-phosphate carboxylase (ssRUBISCO, a very abundant plant polypeptide) and the full-length transcript promoter from the figwort mosaic virus (FMV35S), promoters from the maize ubiquitin and rice actin genes. All of these promoters have been used to create various types of DNA constructs which have been expressed in plants; see, e.g., PCT publication WO 84/02913 (Rogers et al., Monsanto).

Promoters which are known or found to cause transcription of DNA in plant cells can be used in the present invention. Such promoters may be obtained from a variety of sources such as plants and plant DNA viruses and include, but are not limited to, the CaMV35S and FMV35S promoters and promoters isolated from plant genes such as ssRLTBISCO genes and the maize ubiquitin and rice actin genes. As described below, it is preferred that the particular promoter selected should be capable of causing sufficient expression to result in the production of an effective amount of a Class II EPSPS to render the plant substantially tolerant to glyphosate herbicides. The amount of Class II EPSPS needed to induce the desired tolerance may vary with the plant species. It is preferred that the promoters utilized have relatively high expression in all meristematic tissues in addition to other tissues inasmuch as it is now known that glyphosate is translocated and accumulated in this type of plant tissue. Alternatively, a combination of chimeric genes can be used to cumulatively result in the necessary overall expression level of the selected Class II EPSPS enzyme to result in the glyphosate-tolerant phenotype.

The mRNA produced by a DNA construct of the present invention also contains a 5′ non-translated leader sequence. This sequence can be derived from the promoter selected to express the gene, and can be specifically modified so as to increase translation of the MRNA. The 5′ non-translated regions can also be obtained from viral RNAs, from suitable eukaryotic genes, or from a synthetic gene sequence. The present invention is not limited to constructs, as presented in the following examples, wherein the non-translated region is derived from both the 5′ non-translated sequence that accompanies the promoter sequence and part of the 5′ non-translated region of the virus coat protein gene. Rather, the non-translated leader sequence can be derived from an unrelated promoter or coding sequence as discussed above.

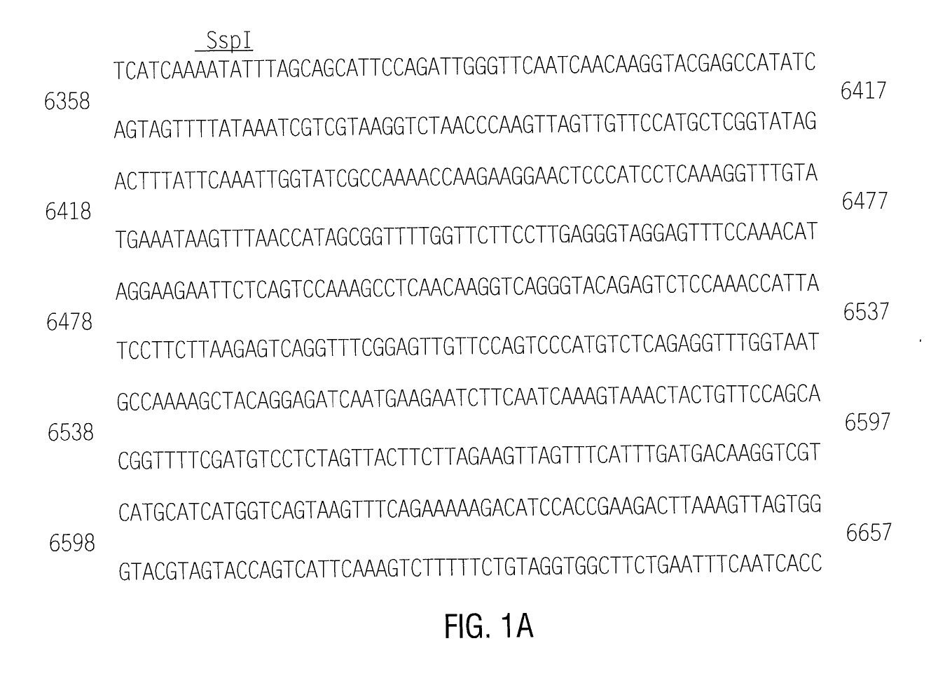

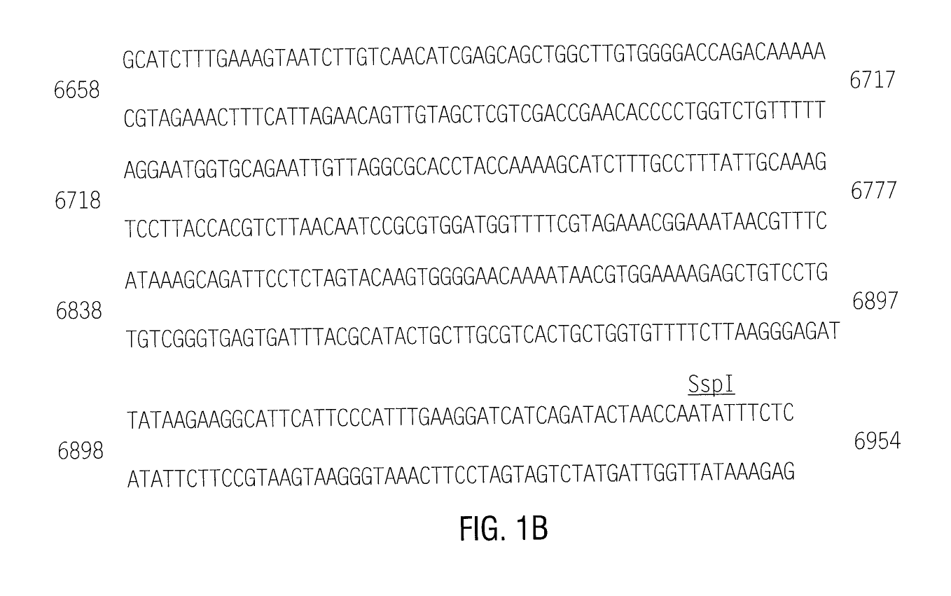

Preferred promoters for use in the present invention the the fill-length transcript (SEQ ID NO:1) promoter from the figwort mosaic virus (FMV35S) and the full-length transcript (35S) promoter from cauliflower mosaic virus (CaMV), including the enhanced CaMV35S promoter (Kay et al. 1987). The FMV35S promoter functions as strong and uniform promoter with particularly good expression in meristematic tissue for chimeric genes inserted into plants, particularly dicotyledons. The resulting transgenic plant in general expresses the protein encoded by the inserted gene at a higher and more uniform level throughout the tissues and cells of the transformed plant than the same gene driven by an enhanced CaMV35S promoter. Referring to FIG. 1, the DNA sequence (SEQ ID NO:1) of the FMV35S promoter is located between nucleotides 6368 and 6930 of the FMV genome. A 5′ non-translated leader sequence is preferably coupled with the promoter. The leader sequence can be from the FMV35S genome itself or can be from a source other than FMV35S.

For expression of heterologous genes in moncotyledonous plants the use of an intron has been found to enhance expression of the heterologous gene. While one may use any of a number of introns which have been isloated from plant genes. the use of the first intron from the maize heat shock 70 gene is preferred.

The 3′ non-translated region of the chimeric plant gene contains a polyadenylation signal which functions in plants to cause the addition of polyadenylate nucleotides to the 3′ end of the viral RNA. Examples of suitable 3′ regions are (1) the 3′ transcribed, non-translated regions containing the polyadenylated signal of Agrobacterium tumor-inducing (Ti) plasmid genes, such as the nopaline synthase (NOS) gene, and (2) plant genes like the soybean storage protein genes and the small subunit of the ribulose-1,5-bisphosphate carboxylase (ssRUBISCO) gene. An example of a preferred 3′ region is that from the ssRUBISCO gene from pea (E9), described in greater detail below.

The DNA constructs of the present invention also contain a structural coding sequence in double-stranded DNA form which encodes a glyphosate-tolerant, highly efficient Class II EPSPS enzyme.

Identification of glyphosatr-tolerant, highly efficient EPSPS enzymes

In an attempt to identify and isolate glyphosate-tolerant, highly efficient EPSPS enzymes, kinetic analysis of the EPSPS enzymes from a number of bacteria exhibiting tolerance to glyphosate or that had been isolated from suitable sources was undertaken. It was discovered that in some cases the EPSPS enzymes showed no tolerance to inhibition by glyphosate and it was concluded that the tolerance phenotype of the bacterium was due to an impermeability to glyphosate or other factors. In a number of cases, however, microorganisms were identified whose EPSPS enzyme showed a greater degree of tolerance to inhibition by glyphosate and that displayed a low Kₘ for PEP when compared to that previously reported for other microbial and plant sources. The EPSPS enzymes from these microorganisms were then subjected to further study and analysis.

Table I displays the data obtained for the EPSPS enzymes identified and isolated as a result of the above described analysis. Table I includes data for three identified Class II EPSPS enzymes that were observed to have a high tolerance to inhibition to glyphosate and a low Kₘ for PEP as well as data for the native Petunia EPSPS and a glyphosate-tolerant variant of the Petunia EPSPS referred to as GA101. The GA101 variant is so named because it exhibits the substitution of an alanine residue for a glycine residue at position 101 (with respect to Petunia). When the change introduced into the Petunia EPSPS (GA101) was introduced into a number of other EPSPS enzymes, similar changes in kinetics were observed, an elevation of the Kᵢ for glyphosate of the Kₘ for PEP.

TABLE I
Kinetic characterization of EPSPS enzymes

| ENZYME | Kₘ PEP | Kᵢ Glyphosate | |
|---|---|---|---|
| SOURCE | (μM) | (μM) | KᵢKₘ |
| Petunia | 5 | 0.4 | 0.08 |
| Petunia GA101 | 200 | 2000 | 10 |
| PG2982 | 2.1-3.1¹ | 25-82 | ˜8-40 |
| LBAA | ˜7.3-8² | 60 (est)⁷ | ˜7.9 |
| CP4 | 12³ | 2720 | 227 |
| B. subtilis 1A2 | 13⁴ | 440 | 33.8 |
| S. aureus | 5⁵ | 200 | 40 |

¹Range of PEP tested = 1-40 μM
²Range of PEP tested = 5-80 μM
³Range of PEP tested = 1.5-40 μM
⁴Range of PEP tested = 1-60 μM
⁵Range of PEP tested = 1-50 μM
⁷(est) = estimated

The Agrobacterium sp. strain CP4 was initially identified by its ability to grow on glyphosate as a carbon source (10 mM) in the presence of 1 mM phosphate. The strain CP4 was identified from a collection obtained from a fixed-bed immobilized cell column that employed Mannville R-635 diatomaceous earth beads. The column had been run for three months on a waste-water feed from a glyphosate production plant. The column contained 50 mg/ml glyphosate and NH3 as NH₄Cl. Total organic carbon was 300 mg/ml and BOD's (Biological Oxygen Demand—a measure of “soft” carbon availability) were less than 30 mg/ml. This treatment column has been described (Heitkamp et al., 1990). Dworkin-Foster minimal salts medium containing glyphosate at 10 nM and with phosphate at 1 mM was used to select for microbes from a wash of this column that were capable of growing on glyphosate as sole carbon source. Dworkin-Foster minimal medium was made up by combining in 1 liter (with autoclaved H₂O), 1 ml each of A, B and C and 10 ml of D (as per below) and thiamine HCI (5 mg).

| | |
|---|---|
| A. | D-F Salts (1000× stock; per 100 ml; autoclaved): |

| | | |
|---|---|---|
| H₃BO₃ | 1 | mg |
| MnSO₄.7H₂O | 1 | mg |
| ZnSO₄.7H₂O | 12.5 | mg |
| CuSO₄.5H₂O | 8 | mg |
| NaMoO₃.3H₂O | 1.7 | mg |

| | | | |
|---|---|---|---|
| B. | FeSO₄.7H₂O (1000× stock: per 100 ml; autoclaved) | 0.1 | g |
| C. | MgSO₄.7H₂O (1000× stock; per 100 ml; autoclaved) | 20 | g |
| D. | (NH₄)₂SO₄ (100× stock; per 100 ml; autoclaved) | 20 | g |

Yeast Extract (YE; Difco) was added to a final concentration of 0.01 or 0.001%. The strain CP4 was also grown on media composed of D-F salts, amended as described above, containing glucose, gluconate and citrate (each at 0.1 %) as carbon sources and with inorganic phosphate (0.2-1.0 mM) as the phosphorous source.

Other Class II EPSPS containing microorganisms were identified as Achromobacter sp. strain LBAA (Hallas et aL., 1988), Pseudomonas sp. strain PG2982 (Moore et al., 1983; Fitzgibbon 1988), Bacillus subtilis IA2 (Henner et al., 1984) and Staphylococcus aureus (O'Connell et al., 1993). It had been reported previously, from measurements in crude lysates, that the EPSPS enzyme from strain PG2982 was less sensitive to inhibition to glyphosate than that of E. coli, but there has been no report of the details of this lack of sensitivity and there has been no report on the Kₘ for PEP for this enzyme or of the DNA sequence for the gene for this enzyme (Fitzgibbon, 1988; Fitzgibbon and Braymer, 1990).

Relationship of the Class II EPSPS to those previously studied

All EPSPS proteins studied to date have shown a remarkable degree of homology. For example, bacterial and plant EPSPS's are about 54% identical and with similarity as high as 80%. Within bacterial EPSPS's and plant EPSPS's themselyes the degree of identity and similarity is much greater (see Table II).

TABLE II
Comparison between exemplary Class I EPSPS
protein sequences¹

| | similarity | identity |
|---|---|---|
| E. coli vs. S. typhimurium | 93 | 88 |
| P. hybrida vs. E. coli | 72 | 55 |
| P. hybrida vs. L. esculentum | 93 | 88 |

¹The EPSPS sequences compared here were obtained from the following references: E. coli. Rogers et al., 1983; S. typhimunum, Stalker et al., 1985; Petunia hybrida, Shah et al., 1986; and tomato (L. esculentum), Gasser et al., 1988.

When crude extracts of CP4 and LBAA bacteria (50 μg protein) were probed using rabbit anti-EPSPS antibody (Padgette et al., 1987) to the Petunia EPSPS protein in a Western analysis, no positive signal could be detected, even with extended exposure times (Protein A-1251 development system) and under conditions where the control EPSPS (Petunia EPSPS, 20 ng; a Class I EPSPS) was readily detected. The presence of EPSPS activity in these extracts was confirmed by enzyme assay. This surprising result, indicating a lack of similarity between the EPSPS's from these bacterial isolates and those previously studied, coupled with the combination of a low Kₘ for PEP and a high Kᵢ for glyphosate, illustrates that these new EPSPS enzymes are different from known EPSPS enzymes (now referred to as Class I EPSPS).

Glyphosate-tolerant Enzymes in Microbial Isolates

For clarity and brevity of disclosure, the following description of the isolation of genes encoding Class II EPSPS enzymes is directed to the isolation of such a gene from a bacterial isolate. Those skilled in the art will recognize that the same or similar strategy can be utilized to isolate such genes from other microbial isolates, plant or fungal sources.

Cloning of the Agrobacterium sp. strain CP4 EPSPS (Gene(s) in E. coli

Having established the existence of a suitable EPSPS in Agrobacterium sp. strain CP4, two parallel approaches were undertaken to clone the gene: cloning based on the expected phenotype for a glyphosate-tolerant EPSPS; and purification of the enzyme to provide material to raise antibodies and to obtain amino acid sequences from the protein to facilitate the verification of clones. Cloning and genetic techniques, unless otherwise indicated, are generally those described in Maniatis et al., 1982 or Sambrook et al., 1987. The cloning strategy was as follows: introduction of a cosmid bank of strain Agrobacterilum sp. strain CP4 into E. coli and selection for the EPSPS gene by selection for growth on inhibitory concentrations of glyphosate.

Chromosomal DNA was prepared from strain Agrobacterium sp. strain CP4 as follows: The cell pellet from a 200 ml L-Broth (Miller, 1972), late log phase culture of Agrobacterium sp. strain CP4 was resuspended in 10 ml of Solution I; 50 mnM Glucose, 10 mini EDTA, 25 mM Tris -CL pH 8.0 (Birnboim and Doly, 1979). SDS was added to a final concentration of 1% and the suspension was subjected to three freeze-thaw cycles, each consisting of immersion in dry ice for 15 minutes and in water at 70° C. for 10 mninutes. The lysate was then extracted four times with equal volumes of phenol:chloroform (1:1; phenol saturated with TE; TE =10 mM Tris pH8.0; 1.0 mM EDTA) and the phases separated by centrifugation (15000g; 10 minutes). The ethanol-precipitable material was pelleted from the supernatant by brief centrifugation (8000 g, 5 minutes) following addition of two volumes of ethanol. The pellet was resuspended in 5 ml TE and dialyzed for 16 hours at 4° C. against 2 liters TE. This preparation yielded a 5 ml DNA solution of 552 μg/ml.

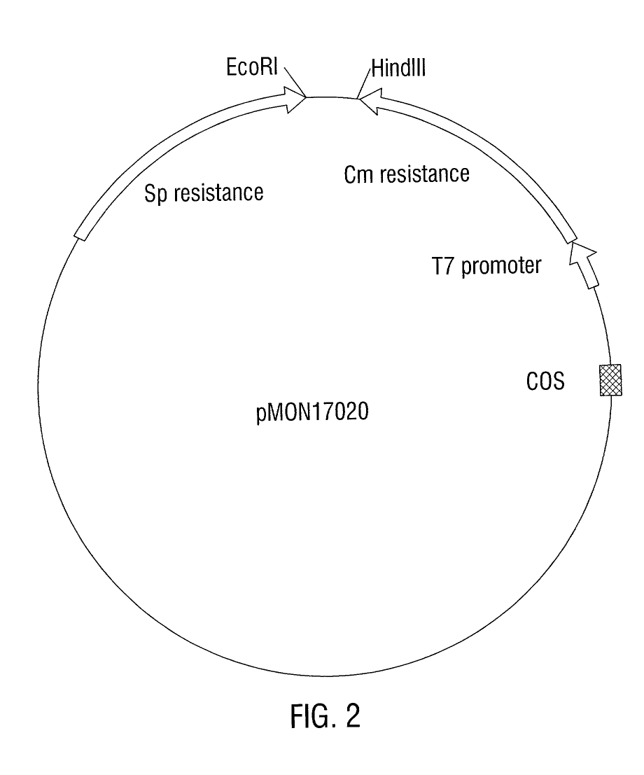

Partially-restricted DNA was prepared as follows. Three 100 μg aliquot samples of CP4 DNA were treated for 1 hour at 37° C. with restriction endonuclease HindIII at rates of 4, 2 and 1 enzyme unit/μg DNA, respectively. The DNA samples were pooled, made 0.25 mM with EDTA and extracted with an equal volume of phenol:chloroform. Following the addition of sodium acetate and ethanol, the DNA was precipitated with two volumes of ethanol and pelleted by centrifugation (12000 g; 10 minutes). The dried DNA pellet was resuspended in 500 μl TE and layered on a 10-40% Sucrose gradient (in 5% increments of 5.5 ml each) in 0.5 M NaCl, 50 mM Tris pH8.0, 5 mM EDTA. Following centrifugation for 20 hours at 26,000 rpm in a SW28 rotor, the tubes were punctured and 1.5 ml fractions collected. Samples (20 μl) of each second fraction were run on 0.7% agarose gel and the size of the DNA determined by comparison with linearized lambda DNA and HindIII-digested lambda DNA standards. Fractions containing DNA of 25-35 kb fragments were pooled, desalted on AMICONIO columns (7000 rpm; 20° C.; 45 minutes) and concentrated by precipitation. This procedure yielded 15 jg of CP4 DNA of the required size. A cosmid bank was constructed using the vector pMON17020. This vector. a map of which is presented in FIG. 2, is based on the pBR327 replicon and contains the spectinomycin/streptomycin (Spr;spc) resistance gene from Tn7 (Fling et al., 1985). the chloramphenicol resistance gene (Cmr;cat) from Tn9 (Alton et al., 1979), the gene10 promoter region from phage T7 (Dunn et al., 1983), and the 1.6 kb BglII phage lambda cos fragment from pHC79 (Hohn and Collins, 1980). A number of cloning sites are located downstream of the cat gene. Since the predominant block to the expression of genes from other microbial sources in E. coli appears to be at the level of transcription, the use of the T7 promoter and supplying the T7 polymerase in trans from the pdP1-2 plasmid (Tabor and Richardson, 1985), enables the expression of large DNA segments of foreign DNA, even those containing RNA polymerase transcription termination sequences. The expression of the spc gene is impaired by transcription from the T7 promoter such that only Cm⁴ can be selected in strains containing pGP1-2. The use of antibiotic resistances such as Cm resistance which do not employ a membrane component is preferred due to the observation that high level expression of resistance genes that involye a membrane component. i.e. J3-lactamase and Amp resistance, give rise to a glyphosate-tolerant phenotype. Presumably, this is due to the exclusion of glyphosate from the cell by the membrane localized resistance protein. It is also preferred that the selectable marker be oriented in the same direction as the T7 promoter.

The vector was then cut with HindIII and treated with calf alkaline phosphatase (CAP) in preparation for cloning. Vector and target sequences were ligated by combining the following:

| | | |
|---|---|---|
| Vector DNA (HindIII/CAP) | 3 | μg |
| Size fractionated CP4 HindIII fragments | 1.5 | μg |
| 10× ligation buffer | 2.2 | μl |
| T4 DNA ligase (New England Biolabs) (400 U/μl) | 1.0 | μl |

and adding H₂O. This mixture was incubated for 18 hours at 16° C. 10× ligation buffer is 250 mM Tris-HCI, pH 8.0; 100 mM MgCI₂; 100 mM Dithiothreitol: 2 mM Spermidine. The ligated DNA (5 μl) was packaged into lambda phage particles (Stratagene; Gigapack Gold) using the manufacturer's procedure.

A sample (200μl) of E. coli HB11 (Boyer and Rolland-Dussoix, 1973) containing the T7 polymerase expression plasmid pGP1-2 (Tabor and Richardson, 1985) and grown overnight in L-Broth (with maltose at 0.2% and kanamycin at 50 μg/ml) was infected with 50 μl of the packaged DNA. Transformants were selected at 30° C. on M9 (Miller, 1972) agar containing kanamycin (50 μg/ml), chloramphenicol (25 μg/ml), L-proline (50 μg/ml), L-leucine (50 μg(ml) and B1 (5 μg/ml), and with glyphosate at 3.0 mM. Aliquot samples were also plated on the same media lacking glyphosate to titer the packaged cosmids. Cosmid transformants were isolated on this latter medium at a rate of ˜-5×10⁵ per fig CP4 HindIII DNA after 3 days at 30° C. Colonies arose on the glyphosate agar from day 3 until day 15 with a final rate of ˜1 per 200 cosmids. DNA was prepared from 14 glyphosate-tolerant clones and, following verification of this phenotype, was transformed into E. coli GB1OOfpGP1-2 (E. coli GB100 is an aroA derivative of MM294 (Talmadge and Gilbert, 19801) and tested for complementation for growth in the absence of added aromatic amino acids and aminobenzoic acids. Other aroA strains such as SR481 (Bachman et al., 1980; Padgette et al., 1987), could be used and would be suitable for this experiment. The use of GB100 is merely exemplary and should not be viewed in a limiting sense. This aroA strain usually requires that growth media be supplemented with L-phenylalanine, L-tyrosine and L-tryptophan each at 100 μg/ml and with para-hydroxybenzoic acid, 2,3-dihydroxybenzoic acid and para-aminobenzoic acid each at 5 μg/ml for growth in minimal media. Of the fourteen cosmids tested only one showed complementation of the aroA-phenotype. Transformants of this cosmid, pMON17076, showed weak but uniform growth on the unsupplemented minimal media after 10 days.

The proteins encoded by the cosmids were determined in uivo using a T7 expression system (Tabor and Richardson, 1985). Cultures of E. coli containing pGP1-2 (Tabor and Richardson, 1985) and test and control cosmids were grown at 30° C. in L-broth (2 ml) with chloramphenicol and kanamycin (25 and 50 μl, respectively) to a Klett reading of 50. An aliquot was removed and the cells collected by centrifugation, washed with M9 salts (Miller, 1972) and resuspended in 1 ml M9 medium containing glucose at 0.2%, thiamine at 20 μg(ml and containing the 18 amino acids at 0.01% (minus cysteine and methionine). Following incubation at 30° C. for 90 minutes, the cultures were transferred to a 42° C. water bath and held there for 15 minutes. Rifampicin (Sigma) was added to 200 μg(ml and the cultures held at 42° C. for 10 additional mminutes and then transferred to 30° C. for 20 minutes. Samples were pulsed with 10 μCi of ³⁵S-methionine for 5 minutes at 30° C. The cells were collected by centrifugation and suspended in 60-120 pi cracking buffer (60 mM Tris-HCI 6.8, 1% SDS, 1% 2-mercaptoethanol, 10% glycerol, 0.01% bromophenol blue). Aliquot samples were electrophoresed on 12.5% SDIS-PAGE and following soaking for 60 minutes in 10 volumes of Acetic Acid-Methanol-water (10:30:60), the gel was soaked in ENLIGHTNING T™ (DUPONT) following manufacturer's directions, dried, and exposed at −70° C. to X-Ray film. Proteins of about 45 kd in size, labeled with 35S-methionine, were detected in number of the cosmids. including pMON17076.

Purification of EPSPS from Arobacterium spa strain CP4

All protein purification procedures were carried out at 3-5° C. EPSPS enzyme assays were performed using either the phosphate release or radioactive HPLC method, as previously described in Padgette et al., 1987, using 1 mM phosphoenol pyruvate (PEP, Boehringer) and 2 mM shikimate-3-phosphate 1S3P) substrate concentrations. For radioactive HPLC assays, 14C-PEP (Amersham) was utilized. S3P was synthesized as previously described in Wibbenmeyer et al. 1988. N-terminal amino acid sequencing was performed by loading samples onto a Polybrene precycled filter in aliquots while drying. Automated Edman degradation chemistry was used to determine the N-terminal protein sequence, using an Applied Biosystems Model 470A gas phase sequencer (Hunkapiller et at., 1983) with an Applied Biosystems 120A PTH analyzer.

Five 100litre fermentations were carried out on a spontaneous “smooth” isolate of strain CP4 that displayed less clumping when grown in liquid culture. This reduced clumping and smooth colony morphology may be due to reduced polysaccharide production by this isolate. In the following section dealing with the purification of the EPSPS enzyme, CP4 refers to the “smooth” isolate—CP4-S1. The cells from the three batches showing the highest specific activities were pooled. Cell paste of Agrobacterium sp. CP4 (300 g) was washed twice with 0.5 L of 0.9% salne and collected by centrifuation (30 minutes, 8000 rpm in a GS3 Sorvall rotor). The cell pellet was suspended in 0.9 L extraction buffer (100 maM TrisCl, 1 mM EDTA, 1 mM BAM (Benzamidine), 5 mM DTT, 10% glycerol, pH 7.5) and lysed by 2 passes through a Manton Gaulin cell. The resulting solution was centrifuiged (30 minutes, 8000 rpm) and the supernatant was treated with 0.21 L of 1.5% protamine sulfate (in 100 rmM TrisCl, pH 7.5, 0.2% w/v final protamine sulfate concentration). After stirring for 1 hour, the m e was centrifuged (50 minutes, 8000 rpm) and the resulting supernatant treated with solid ammonium sulfate to 40% saturation and stirred for I hour. After centrifugation (50 minutes, 8000 rpm), the resulting supernatant was treated with solid ammonium sulfate to 70% saturation, stirred for 50 minutes, and the insoluble protein was collected by centrifugation (1 hour, 8000 rpm). This 40-70% ammonium sulfate fraction was then dissolyed in extraction buffer to give a final volume of 0.2 L, and dialyzed twice (Spectrum 10,000 MW cutoff dialysis tubing) against 2 L of extraction buffer for a total of 12 hours.

To the resulting dialyzed 40-70% ammonium sulfate fraction tO.29 L) was added solid ammonium sulfate to give a final concentration of 1 M. This material was loaded (2 ml/min) onto a coluimn (5 cm×15 cm, 295 ml) packed with phenyl Sepharose CL-4B (Pharmacia) resin equilibrated with extraction buffer containing 1 M ammonium sulfate, and washed with the same buffer (1.5 L, 2 ml/min). EPSPS was eluted with a linear gradient of extraction buffer going from 1 M to 0.00 M ammonium sulfate (total volume of 1.5 L, 2 ml/min). Fractions were collected (20 ml) and assayed for EPSPS activity by the phosphate release assay. The fractions with the highest EPSPS activity (fractions 3650) were pooled and dialyzed against 3×2 L (18 hours) of 10 mM TrisCl, 25 mM KCr, 1 mM EDTA, 5 mM DTT, 10% glycerol, pH 7.8.

The dialyzed EPSPS extract (350 ml) was loaded (5 ml/min) onto a column (2.4 cm×30 cm, 136 ml) packed with Q-Sepharose Fast Flow (Pharmacia) resin equilibrated with 10 mM TrisCl, 25 mM KCR, 5 mM DTT, 10% glycerol, pH 7.8 (Q Sepharose buffer), and washed with 1 L of the same buffer. EPSPS was eluted with a linear gradient of Q Sepharose buffer going from 0.025 M to 0.40 M KCI (total volume of 1.4 L, 5 ml/min). Fractions were collected (15 ml) and assayed for EPSPS activity by the phosphate release assay. The fractions with the highest EPSPS activity (fractions 47-60) were pooled and the protein was precipitated by adding solid ammonium sulfate to 80% saturation and stirring for 1 hour. The precipitated protein was collected by centrifugation (20 minutes, 12000 rpm in a GSA Sorvall rotor), dissolyed in Q Sepharose buffer (total volume of 14 ml), and dialyzed against the same buffer (2×1 L, 18 hours).

The resulting dialyzed partially purified EPSPS extract (19 ml) was loaded (1.7 ml/min) onto a Mono Q 10/10 column (Pharmacia) equilibrated with Q Sepharose buffer, and washed with the same buffer t35 ml). EPSPS was eluted with a linear gradient of 0.025 M to 0.35 M KCI (total volume of 119 ml, 1.7 ml/min). Fractions were collected (1.7 ml) and assayed for EPSPS activity by the phosphate release assay. The fractions with the highest EPSPS activity (fractions 30-37) were pooled (6 ml).

The Mono Q pool was made 1 M in ammonium sulfate by the addition of solid ammonium sulfate and 2 ml aliquots were chromatographed on a Phenyl Superose 5/5 column (Pharmacia) equilibrated with 100 mM TrisCl, 5 mM DTT, 1 M ammonium sulfate, 10% glycerol, pH 7.5 (Phenyl Superose buffer). Samples were loaded (1 ml/min), washed with Phenyl Superose buffer (10 ml), and eluted with a linear gradient of Phenyl Superose buffer going from 1 M to 0.00 M ammonium sulfate (total volume of 60 ml, 1 ml/min). Fractions were collected (1 ml) and assayed for EPSPS activity by the phosphate release assay. The fractions from each run with the highest EPSPS activity (fractions ˜36-40) were pooled together (10 ml, 2.5 mg protein). For N-terminal amino acid sequence determination, a portion of one fraction (#39 from run 1) was dialyzed against 50 mM NaHCO₃ (2×1 L). The resulting pure EPSPS sample (0.9 ml, 77 μg protein) was found to exhibit a single N-terminal amino acid sequence of:

XH(G)ASSRPATARKSS(G)LX(G)(T)VR)IPG(D)(K)(M) (SEQ ID NO:18).

The remaining Phenyl Superose EPSPS pool was dialyzed against 50 mM TrisCl, 2 mM DIT, 10 mM KCl, 10% glycerol, pH 7.5 (2×1 L). An aliquot (0.55 ml, 0.61 mg protein) was loaded (1 ml/min) onto a Mono Q 5/5 column (Pharmacia) equilibrated with Q Sepharose buffer, washed with the same buffer (5 ml), and eluted with a linear gradient of Q Sepharose buffer going from 0-0.14 M KCI in 10 minutes, then holding at 0.14 M KCl (1 ml/min). Fractions were collected (1 ml) and assayed for EPSPS activity by the phosphate release assay and were subjected to SDS-PAGE (10-15%, Phast System, Pharmacia, with silyer staining) to determine protein purity. Fractions exhibiting a single band of protein by SDS-PAGE (22-25, 222 Ag) were pooled and dialyzed against 100 mM ammonium bicarbonate, pH 8.1 (2×1 L, 9 hours).

Trysinolysis and neotide seguencinz of Aarobacterium ST strain CP4 EPSPS

To the resulting pure Agrobacterium sp. strain CP4 EPSPS (111 μg) was added 3 μg of trypsin (Calbiochem), and the trypsinolysis reaction was allowed to proceed for 16 hours at 37° C. The tryptic digest was then chromatographed (lml/min) on a C18 reverse phase HPLC column (Vydac) as previously described in Padgette et al., 1988 for E. coli EPSPS. For all peptide purifications, 0.1% trifluoroacetic acid (TFA, Pierce) was designated buffer “RP--A” and 0.1% TFA in acetonitrile was buffer “RP-B”. The gradient used for elution of the trypsinized Agrobacterium sp. CP4 EPSPS was: 0-8 minutes, 0% RP-B; 8-28 minutes, 0-15% RP-B; 28-40 minutes, 15-21% RP-B; 40-68 minutes, 21-49% RP-B; 68-72 minutes, 49-75% RP-B; 72-74 minutes, 75-100% RP-B. Fractions were collected (1 ml) and, based on the elution profile at 210 nm, at least 70 distinct peptides were produced from the trypsinized EPSPS. Fractions 40-70 were evaporated to dryness and redissolyed in 150 μl each of 10% acetonitrile, 0.1% trifluoroacetic acid.

The fraction 61 peptide was further purified on the C18 column by the gradient: 0-5 minutes, 0% RP-B; 5-10 minutes, 0-38% RP-B; 10-30 minutes, 38-45% B. Fractions were collected based on the UV signal at 210 nm. A large peptide peak in fraction 24 eluted at 42% RP-B and was dried down, resuspended as described above, and rechromatographed on the C18 column with the gradient: 0-5 minutes, 0% RP-B; 5-12 min, 0-38% RP-B; 12-15 min, 38-39% RP-B; 15-18 minutes, 39% RP-B; 18-20 minutes. 39-41% RP-B; 20-24 minutes, 41% RP-B; 24-28 minutes, 42% RP-B. The peptide in fraction 25, eluting at 41% RP-B and designated peptide 61-24-25, was subjected to N-terminal amino acid sequencing, and the following sequence was determined:

APSM(I)(D)EYPILAV (SEQ ID NO:19)

The CP4 EPSPS fraction 53 tryptic peptide was further purified by C18 HPLC by the gradient 0% B (5 minutes). 0-30% B (5-17 minutes), 30-40% B (17-37 minutes). The peptide in fraction 28. eluting at 34% B and designated peptide 53-28, was subjected to N-terminal amino acid sequencing, and the following sequence was determined:

ITGLLEGEDVINTGK (SEQ ID NO:20).

In order to verify the CP4 EPSPS cosmid clone, a number of oligonucleotide probes were designed on the basis of the sequence of two of the tryptic sequences from the CP4 enzyme (Table III). The probe identified as MID was very low degeneracy and was used for initial screening. The probes identified as EDV-C and EDV-T were based on the same amino acid sequences and differ in one position (underlined in Table III below) and were used as confirmatory probes, with a positive to be expected only from one of these two probes. In the oligonucleotides below, alternate acceptable nucleotides at a particular position are designated by a “/” such as A(C/T.

TABLE III
Selected CP4 EPSPS peptide seaquences and DNA probes

| | |
|---|---|
| PEPTIDE 61-24-25 APSM(I)(D)EYPILAV | (SEQ ID NO:19) |
| Probe MID; 17-mer; mixed probe; 24-fold degenerate | |
| ATGATA/C/TGAC/TGAG/ATAC/TCC | (SEQ ID NO:21) |
| PEPTIDE 53-28 ITGLLEGEDVINTGK | (SEQ ID NO:20) |
| Probe EDV-C; 17-mer; mixed probe; 48-fold degenerate | |
| GAA/GGAC/TGTA/C/G/TATA/C/TAACAC | (SEQ ID NO:22) |
| Probe EDV-T; 17-mer; mixed probe; 48-fold degenerate | |
| GAA/GGAC/TGTA/C/G/TATA/C/TAATAC | (SEQ ID NO:23) |

The probes were labeled using gamma-32P-ATP and polynucleotide kinase. DNA from fourteen of the cosmids described above was restricted with EcoRI, transferred to membrane and probed with the oligonucleotide probes. The conditions used were as follows: prehybridization was carried out in 6X SSC, 10X Denhardt's for 2-18 hour periods at 60° C., and hybridization was for 48-72 hours in 6X SSC. 10X Denhardt's, 100 utgml tRNA at 100C below the T_{d} for the probe. The T_{d} of the probe was approximated by the formula 20° C.×(A+T) +4° C.×(G+C). The filters were then washed three times with 6X SSC for ten minutes each at room temperature, dried and autoradiographed. Using the MID probe, an ˜9.9 kb fragment in the pMON17076 cosmid gave the only positive signal. This cosmid DNA was then probed with the EDVC (SEQ ID NO:22) and EDV-T (SEQ ID NO:23) probes separately and again this ˜9.9 kb band gave a signal and only with the EDV-T probe.

The combihed data on the glyphosate-tolerant phenotype, the complementation of the E. coli aroA-phenotype, the expression of a -45 Kd protein, and the hybridization to two probes derived from the CP4 EPSPS amino acid sequence strongly suggested that the pMON17076 cosmid contained the EPSPS gene.

Localization and subcloning of the CP4 EPSPS gene

The CP4 EPSPS gene was further localized as follows: a number of additional Southern analyses were carried out on different restriction digests of pMON17076 using the MID (SEQ ID NO:21) and EDV-T (SEQ ID NO:23) probes separately. Based on these analyses and on subsequent detailed restriction mapping of the pBlueScript (Stratagene) subclones of the ˜9.9 kb fragment from pMON17076, a 3.8 kb EcoRI-Sall fragment was identified to which both probes hybridized. This analysis also showed that MID (SEQ ID NO:21) and EDV-T (SEQ ID NO:23) probes hybridized to different sides of BamHI, ClaI, and SacII sites. This 3.8 kb fragment was cloned in both orientations in pBlueScript to form pMON17081 and pMON17082. The phenotypes imparted to E. coli by these clones were then determined. Glyphosate tolerance was determined following transformation into E. coli MM294 containing pGP1-2 (pBlueScript also contains a T7 promoter) on M9 agar media containing glyphosate at 3 mM. Both pMON17081 and pMON17082 showed glyphosate-tolerant colonies at three days at 30° C. at about half the size of the controls on the same media lacking glyphosate. This result suggested that the 3.8 kb fragment contained an intact EPSPS gene. The apparent lack of orientation-dependence of this phenotype could be explained by the presence of the T7 promoter at one side of the cloning sites and the lac promoter at the other. The aroA phenotype was determined in transformants of E. coli GB100 on M9 agar media lacking aromatic supplements. In this experiment, carried out with and without the Plac inducer IPTG, pMON17082 showed much greater growth than pMON17081, suggesting that the EPSPS gene was expressed from the Sall site towards the EcoRi site.

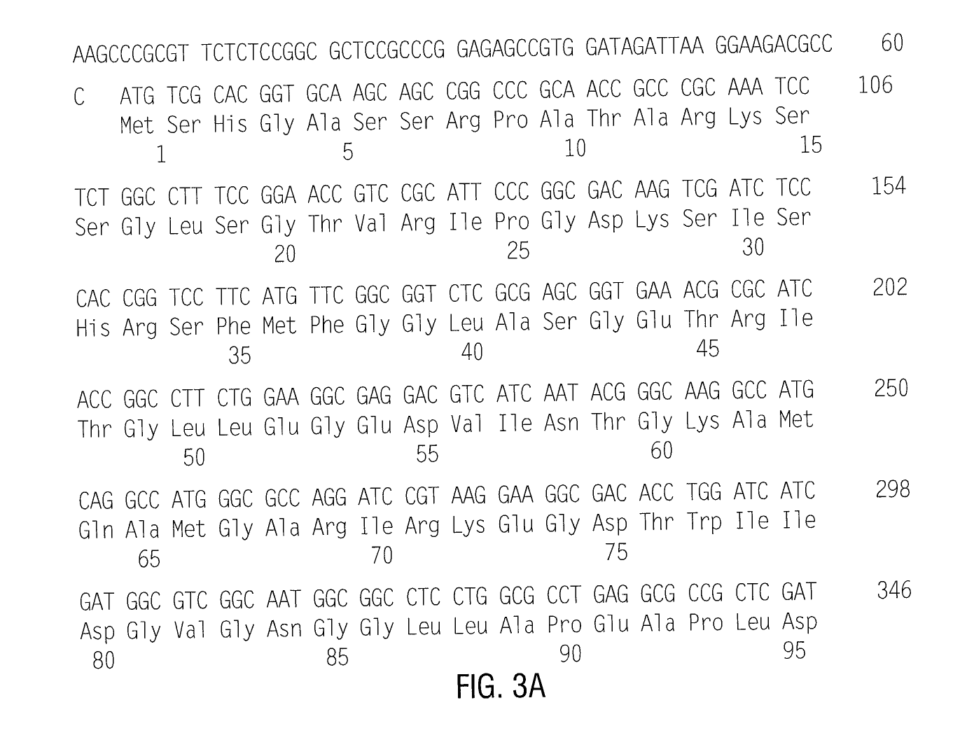

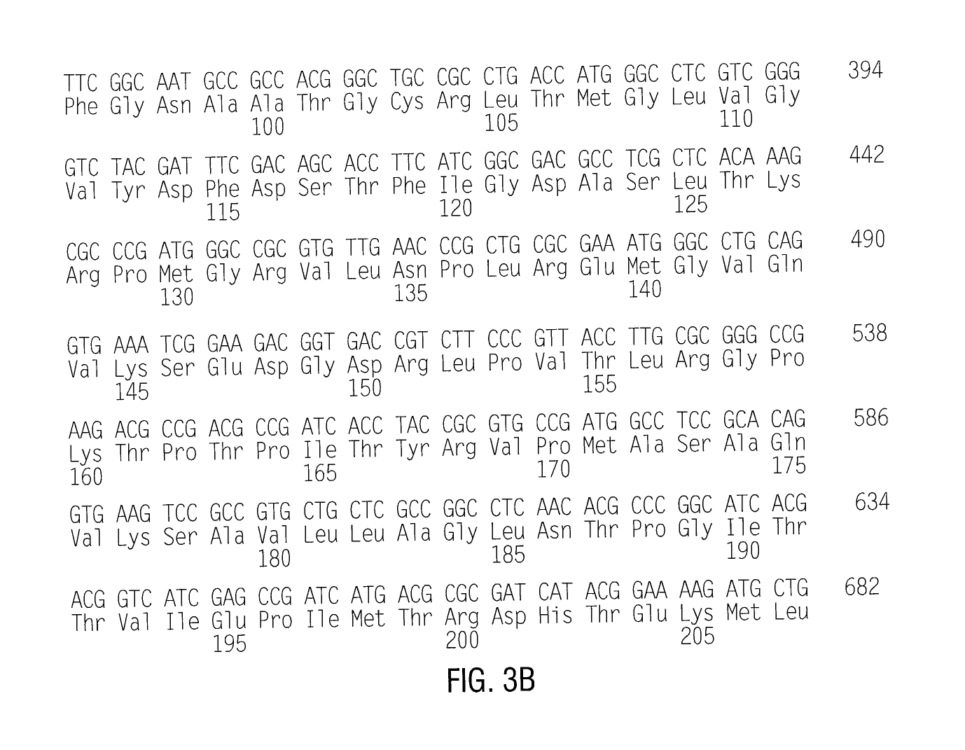

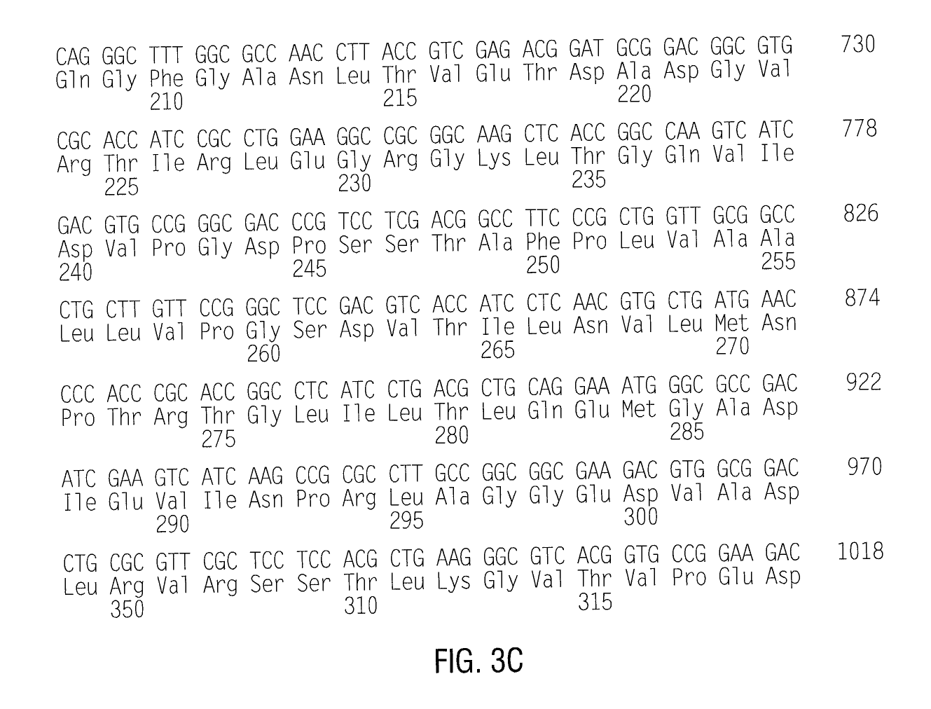

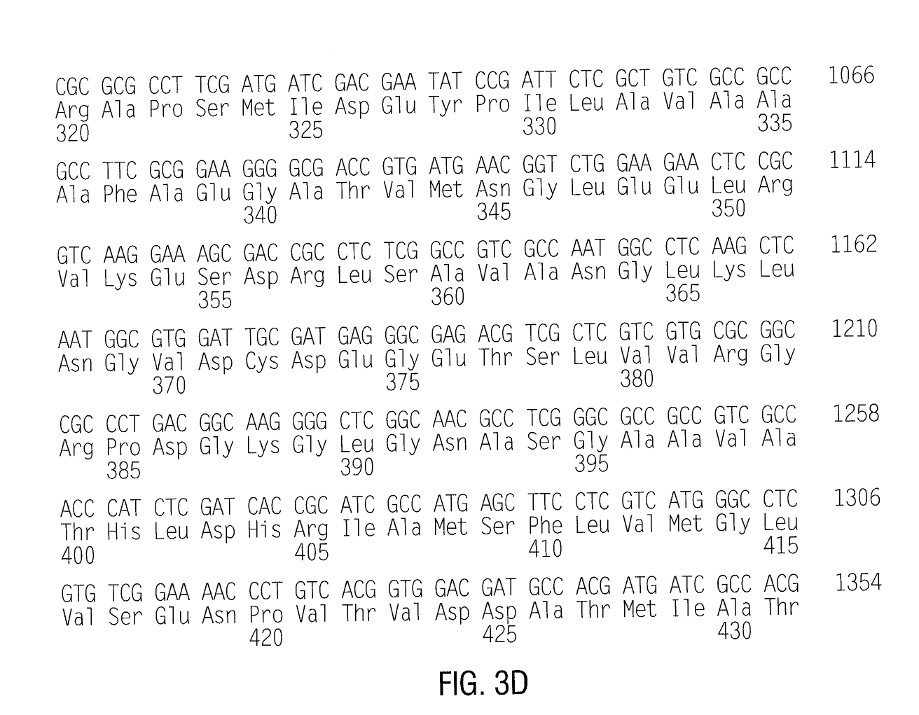

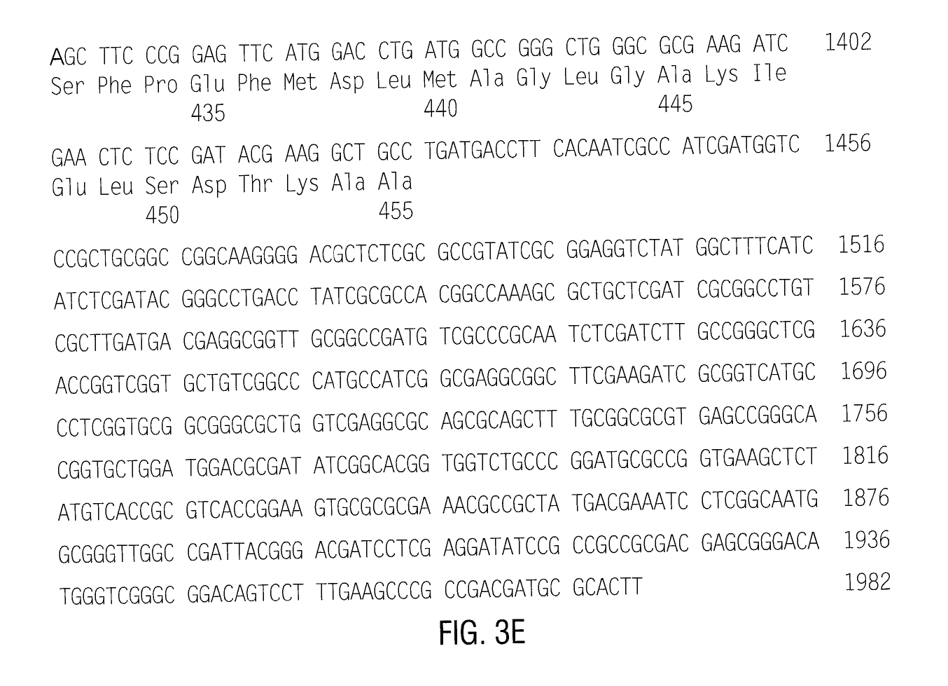

Nucleotide sequencing was begun from a number of restriction site ends, including the BamHI site discussed above. Sequences encoding protein sequences that closely matched the N-terminus protein sequence and that for the tryptic fragment 53-28 (SEQ ID NO:20) (the basis of the EDV-T probe) (SEQ ID NO:23) were localized to the SalI side of this BamHI site. These data provided conclusive evidence for the cloning of the CP4 EPSPS gene and for the direction of transcription of this gene. These data coupled with the restriction mapping data also indicated that the complete gene was located on an ˜2.3 kb XhoI fragment and this fragment was subeloned into pBlueScript. The nucleotide sequence of almost 2 kb of this fragment was determined by a combination of sequencing from cloned restriction fragments and by the use of specific primers to extend the sequence. The nucleotide sequence of the CP4 EPSPS gene and flanking regions is shown in FIG. 3 (SEQ ID NO:2). The sequence corresponding to peptide 61-24-25 (SEQ ID NO:19) was also located. The sequence wvas determined using both the SEQUENASE™ kit from IBI (International Biotechnologies Inc.) and the T7 sequencing/Deaza Kᵢ t from Pharmacia.

That the cloned gene encoded the EPSPS activity purified from the Agrobacterium sp. strain CP4 was verified in the following manner: By a series of site directed mutageneses, BglII and NcoI sites were placed at the N-terminus with the fMet contained within the NcoI recognition sequence, the first internal VcoI site was removed (the-second internal NcoI site was removed later), and a Sacl site was placed after the stop codons. At a later stage the internal NotI site was also removed by site-directed mutagenesis. The following list includes the primers for the site-directed mutagenesis (addition or removal of restriction sites) of the CP4 EPSPS gene. Mutagenesis was carried out by the procedures of Kunkel et al. (1987), essentially as described in Sambrook et al. (1989).

PRIMER BgNc (addition of BglII and NcoI sites to N-terminus)
CGTGGATAGATCTAGGAAGACAACCATGGCTCACGGTC
(SEQ ID NO:24)
PRIMER Sph2 (addition of SphI site to N-terminus)
GGATAGATTAAGGAAGACGCGCATGCTTCACGGTGCAAGCAGCC
(SEQ ID NO:25)
PRIMER S1 (addition of SacI site immediately after stop codons)
GGCTGCCTGATGAGCTCCACAATCGCCATCGATGG
(SEQ ID NO:26)
PRIMER N1 (removal of internal NotI recognition site)
CGTCGCTCGTCGTGCGTGGCCGCCCTGACGGC
(SEQ ID NO:27)
PRIMER Nco1 (removal of first internal NcoI recognition site)
CGGGCAAGGCCATGCAGGCTATGGGCGCC
(SEQ ID NO:28)
PRIMER Nco2 (removal of second internal NcoI recognition site)
CGGGCTGCCGCCTGACTATGGGCCTCGTCGG
(SEQ ID NO:29)

This CP4 EPSPS gene was then cloned as a NcoI-BamER N-terminal fragment plus a BamHI-SacI C-terminal fragment into a PrecA-gene10L expression vector similar to those described (Wong et al., 1988; Olins et al., 1988) to form pMON17101. The Kₘ for PEP and the K, for glyphosate were determined for the EPSPS activity in crude lysates of pMON17101/GB100 transformants following induction with nalidixic acid (Wong et al., 1988) and found to be the same as that determined for the purified and crude enzyme preparations from Agrobacterium sp. strain CP4.

Characterization of the EPSPS vene from Achromobacter sp. strain LBAA and from Pseudomonas sp. strain PG2982

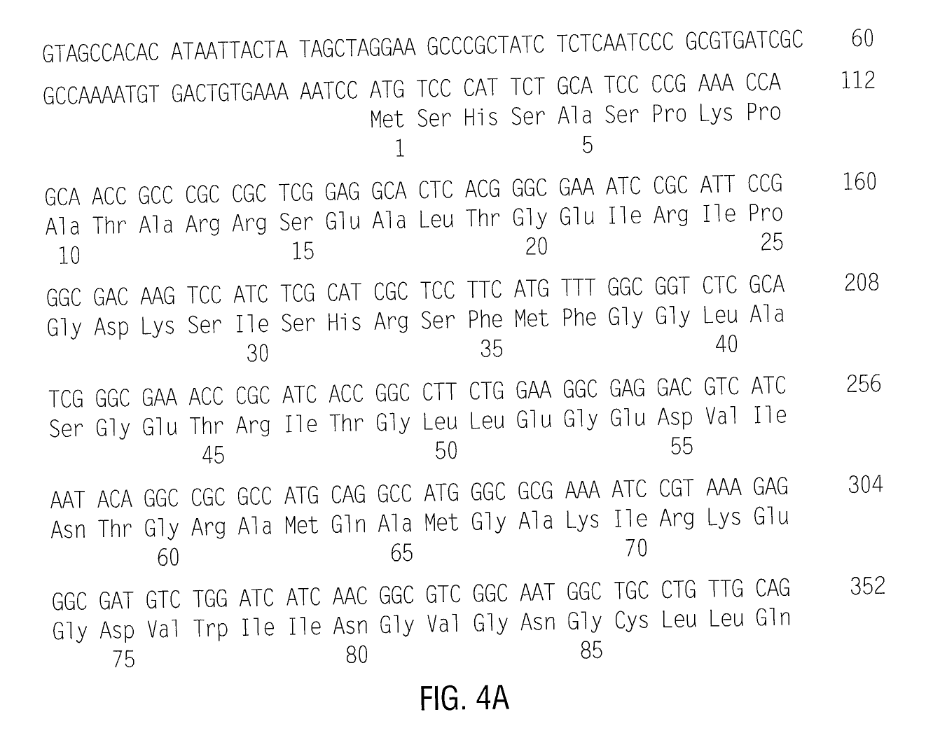

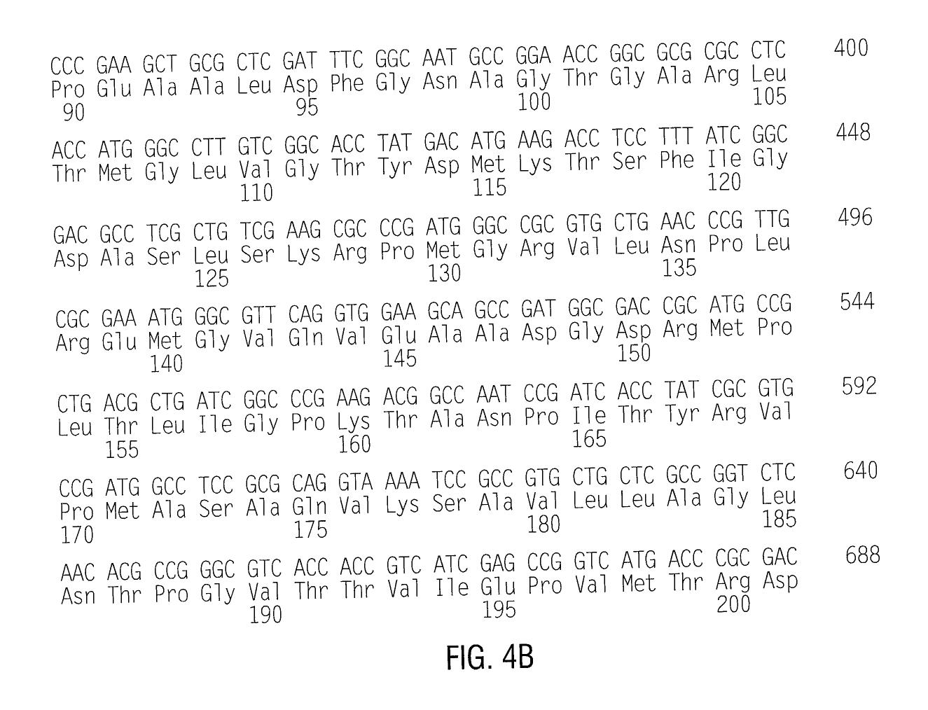

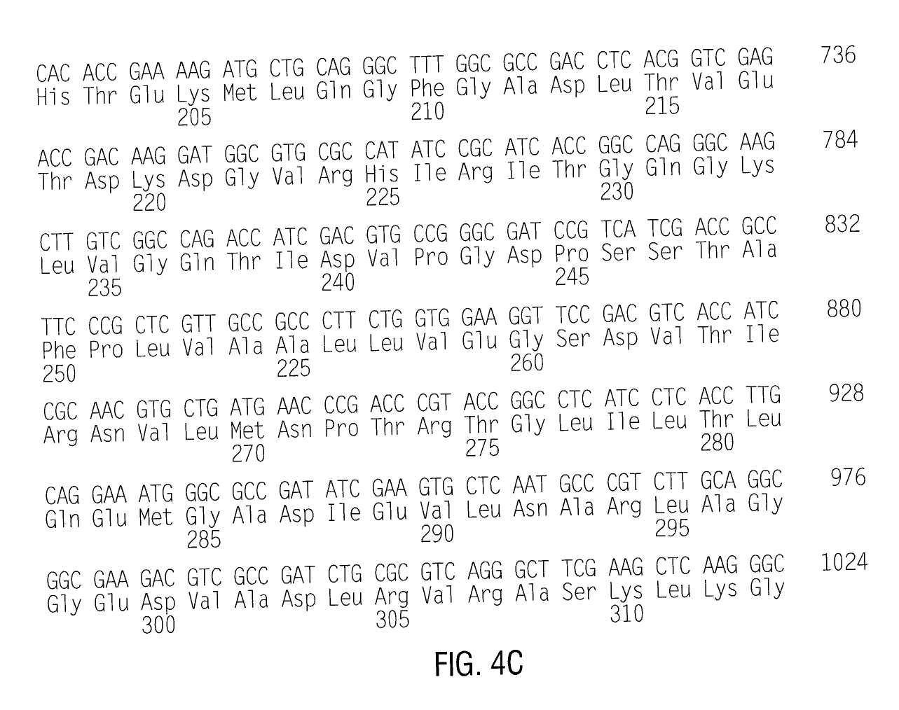

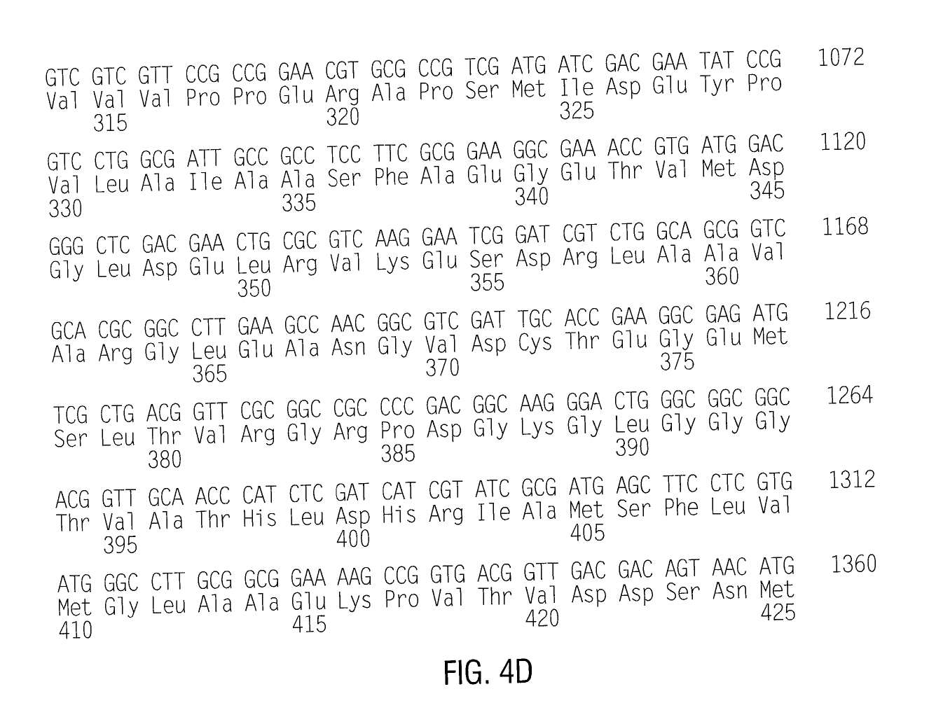

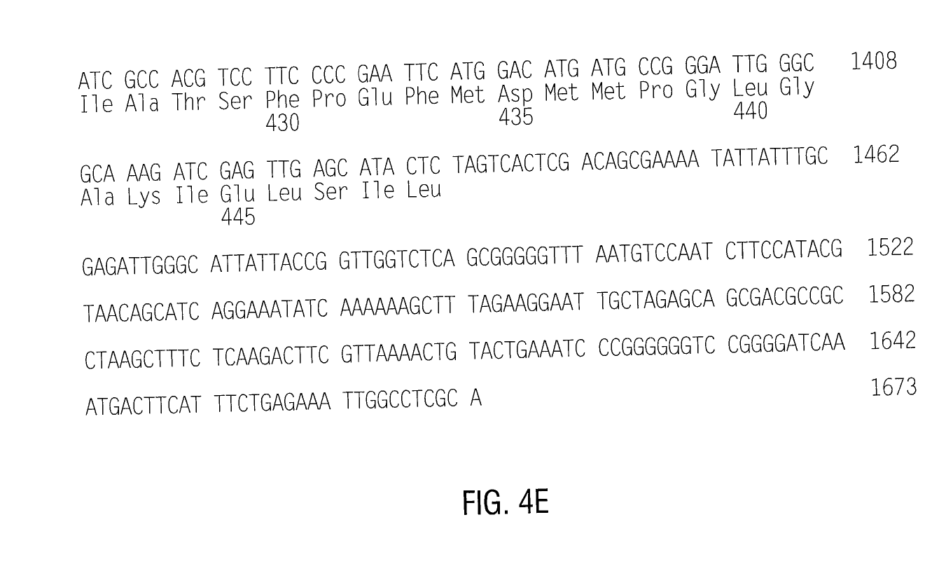

A cosmid bank of partially HindIII-restricted LBAA DNA was constructed in E. coli MM294 in the vector pHC79 (Hohn and Collins, 1980). This bank was probed with a full length CP4 EPSPS gene probe by colony hybridization and positive clones were identified at a rate of ˜1 per 400 cosmids. The LBAA EPSPS gene was further localized in these cosmids by Southern analysis. The gene was located on an ˜2.8 kb Xhol fragment and by a series of sequencing steps, both from restriction fragment ends and by using the oligonucleotide primers from the sequencing of the CP4 EPSPS gene, the nucleotide sequence of the LBAA EPSPS gene was completed and is presented in FIG. 4 (SEQ ID NO:4).

The EPSPS gene from PG2982 was also cloned. The EPSPS protein was purified, essentially as described for the CP4 enzyme, with the following differences: Following the Sepharose CL-4B column, the fractions with the highest EPSPS activity were pooled and the protein precipitated by adding solid ammonium sulfate to 85% saturation and stirring for 1 hour. The precipitated protein was collected by centrifugation, resuspended in Q Sepharose buffer and following dialysis against the same buffer was loaded onto the column (as for the CP4 enzyme). After purification on the Q Sepharose column, ˜40 mg of protein in 100 mnM Tris pH 7.8, 10% glycerol, 1 mM EDTA, 1 mM DTT, and 1 M ammonium sulfate, was loaded onto a Phenyl Superose (Pharmacia) column. The column was eluted at 1.0 ml/minutes with a 40 ml gradient from 1.0 M to 0.00 M ammonium sulfate in the above buffer.

Approximately 1.0 mg of protein from the active fractions of the Phenyl Superose 10/10 column was loaded onto a Pharmacia Mono P 5/10 Chromatofocusing column with a flow rate of 0.75 ml/minutes. The starting buffer was 25 mM bis-Tris at pH 6.3, and the column was eluted with 39 ml of Polybuffer 74, pH 4.0. Approximately 50 g of the peak fraction from the Chromatofocusing column was dialyzed into 25 mM ammonium bicarbonate. This sample was then used to determine the N-terminal amino acid sequence.

The N-terminal sequence obtained was:

XHSASPKPATARRSE (where X an unidentified residue) (SEQ ID NO:30)

A number of degenerate oligonucleotide probes were designed based on this sequence and used to probe a library of PG2982 partial-HindIII DNA in the cosmid pHC79 (Hohn and Collins, 1980) by colony hybridization under nonstringent conditions. Final washing conditions were 15 minutes with 1X SSC, 0.1% SDS at 55° C. One probe with the sequence GCGGTBGCSGGYTrSGG (where B═C, G, or T; S═C or G, and Y═C or T) (SEQ ID NO:31) identified a set of cosmid clones.

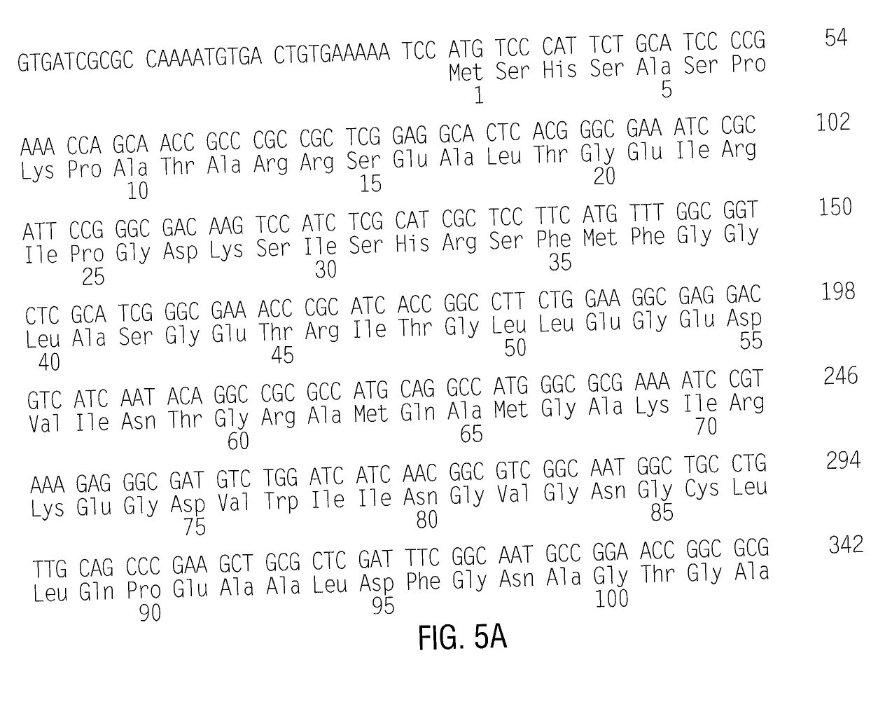

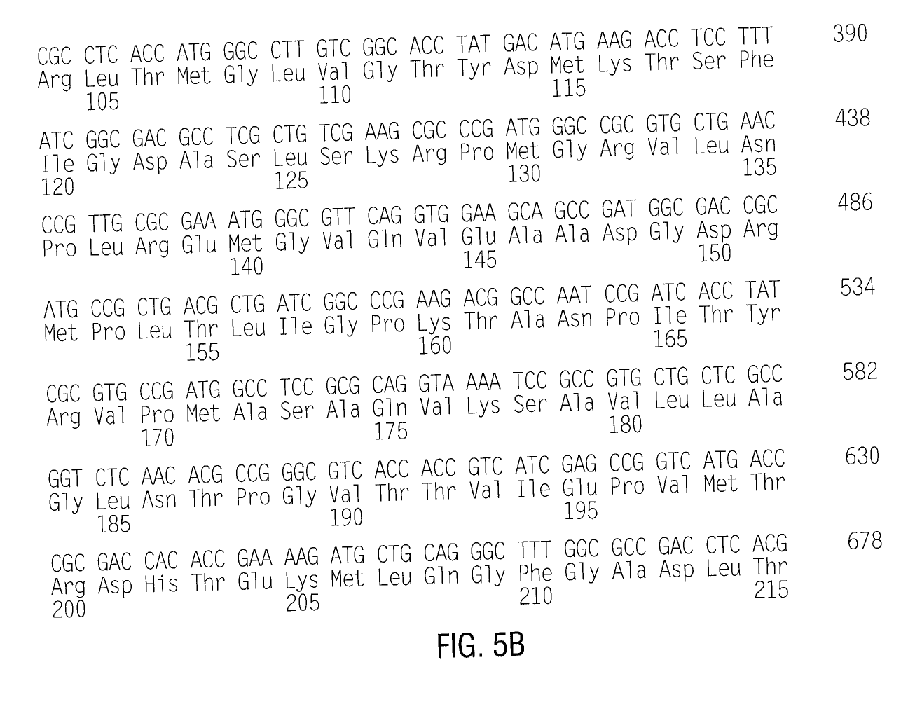

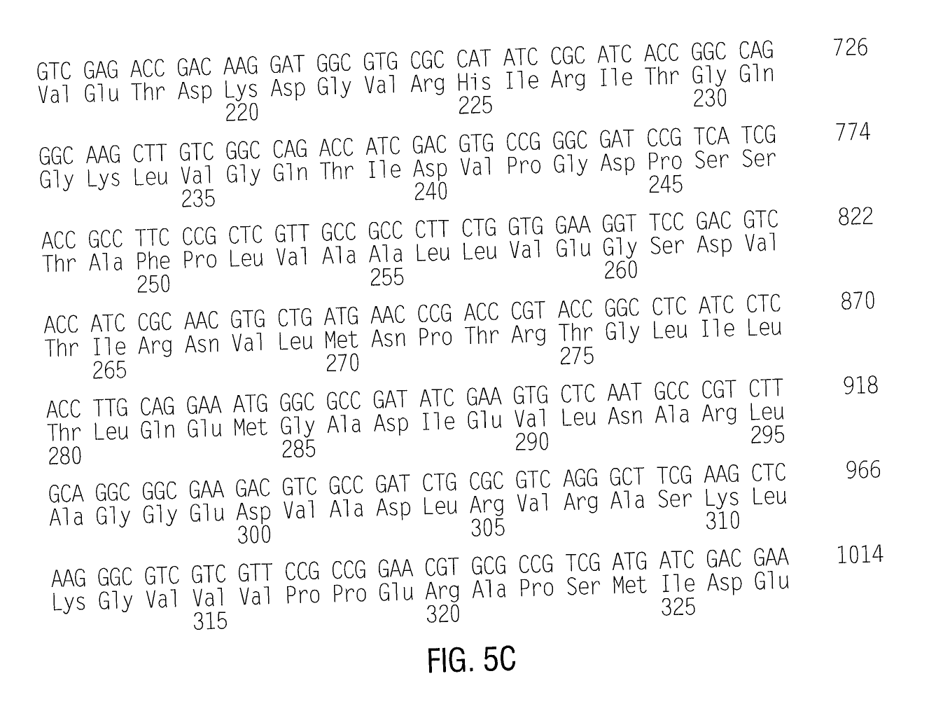

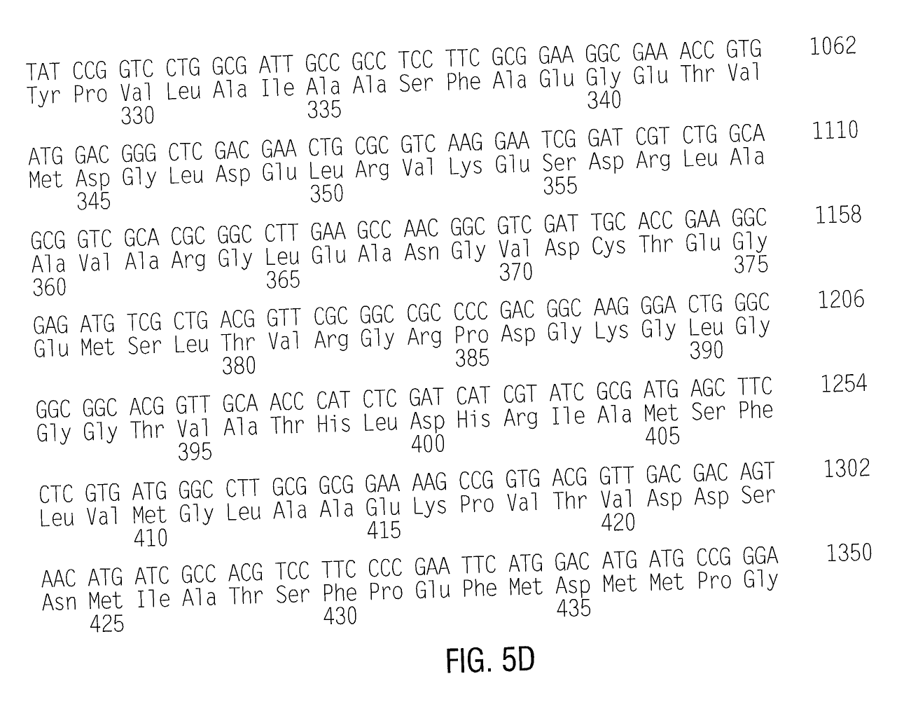

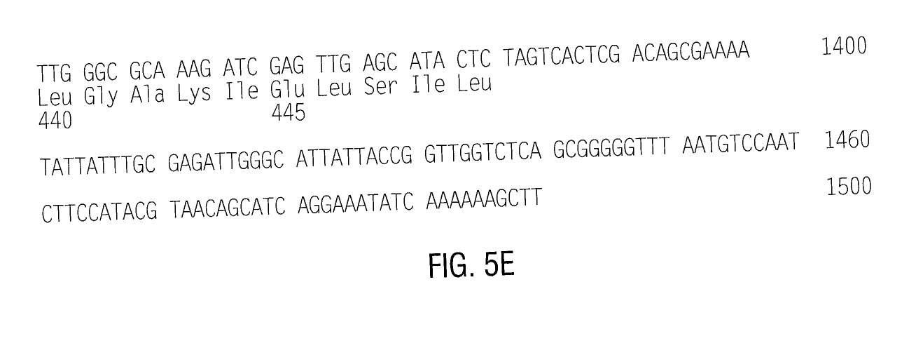

The cosmid set identified in this way was made up of cosmids of diverse HindIII fragments. However, when this set was probed with the CP4 EPSPS gene probe, a cosmid containing the PG2982 EPSPS gene was identified (designated as cosmid 9C1 originally and later as pMON20107). By a series of restriction mappings and Southern analysis this gene was localized to a ˜2.8 kb XhoI fragment and the nucleotide sequence of this gene was determined. This DNA sequence (SEQ ID NO:6) is shown in FIG. 5. There are no nucleotide differences between the EPSPS gene sequences from LBAA (SEQ ID NO:4) and PG2982 (SEQ ID NO:6). The kinetic parameters of the two enzymes are within the range of experimental error.

A gene from PG2982 that imparts glyphosate tolerance in E. coli has been sequenced rFitzgibbon, 1988; Fitzgibbon and Braymer, 1990). The sequence of the PG2982 EPSPS Class II gene shows no homology to the previously reported sequence suggesting that the glyphosate-tolerant phenotype of the previous work is not related to EPSPS.

Charcterization of he EPSPS from Bacillus subtilis

Bacillus subtilis 1A2 (prototroph) was obtained from the Bacillus Genetic Stock Center at Ohio State University. Standard EPSPS assay reactions contained crude bacterial extract with, 1 mM phosphoenolpyruvate (PEP), 2 mM shikimate-3-phosphate (S3P), 0.1 mM ammonium molybdate, 5 mM potassium fluoride, and 50 mM HEPES, pH 7.0 at 25° C. One unit (U) of EPSPS activity is defined as one 1 mol EPSP formed per minute under these conditions. For kinetic determinations, reactions contained crude bacterial, 2 mM S3P, varying concentrations of PEP, and 50 pmM HEPES, pH 7.0 at 25° C. The EPSPS specific activity was found to be 0.003 U/mg. When the assays were performed in the presence of 1 mM glyphosate, 100% of the EPSPS activity was retained. The appKm(PEP) of the B. subtilis EPSPS was determined by measuring the reaction velocity at varying concentrations of PEP. The results were analyzed graphically by the hyperbolic, Lineweaver-Burk and Eadie-Hofstee plots. which yielded appKr(PEP) values of 15.3 μM, 10.8 LM and 12.2 μM, respectively. These three data treatments are in good agreement, and yield an average value for appKₘ(PEP) of 13 μM. The appKᵢ(glyphosate) was estimated by determining the reaction rates of B. subtilis 1A2 EPSPS in the presence of several concentrations of glyphosate, at a PEP concentration of 2 μM. These results were compared to the calculated Vₘₐₓ of the EPSPS, and making the assumption that glyphosate is a competitive inhibitor versus PEP for B. subtilis EPSPS, as it is for all other characterized EPSPSs, an app(glyphosate) was determined graphically. The appK;(glyphosate) was found to be 0.44 mM.

The EPSPS expressed from the B. subtilis aroE gene described by Henner et al. (1986) was also studied. The source of the B. subtilis aroE (EPSPS) gene was the E. coli plasmid-bearing strain ECE13 (original code=MM294[trp100]; Henner, et al., 1984; obtained from the Bacillus Genetic Stock Center at Ohio State University; the culture genotype is [pBR322 trp100] Ap [in MM294] [pBR322::6 kb insert with trpFBA-hisH]). Two strategies were taken to express the enzyme in E. coli GB100 (aroA-): 1) the gene was isolated by PCR and cloned into an overexpression vector, and 2) the gene was subcloned into an overexpression vector. For the PCR cloning of the B. subtilis aroE from ECE13, two oligonucleotides were synthesized which incorporated two restriction enzyme recognition sites (NdeI and E,coRI) to the sequences of the following oligonucleotides:

| | |
|---|---|
| GGAACATATGAAACGAGATAAGGTGCAG | (SEQ ID NO:45) |
| GGAATTCAAACTTCAGGATCTTGAGATAGAAAATG | (SEQ ID NO:46) |

The other approach to the isolation of the B. subtilis aroE gene, subcloning from ECE13 into pUC118, was performed as follows:

(i) Cut ECE13 and pUC with XmaI and SphI.

(ii) Isolate 1700bp aroE fragment and 2600bp pUC118 vector fragment.

(iii) Ligate fragments and transform into GB100.

The subclone was designated pMON21133 and the PCR-derived clone was named pMON21132. Clones from both approaches were first confirmed for complementation of the aroA mutation in E. coli GB100. The cultures exhibited EPSPS specific activities of 0.044 U/mg and 0.71 U/mg for the subdlone (pMON21133) and PCR-derived clone (pMON21132) enzymes, respectively. These specific activities reflect the expected types of expression levels of the two vectors. The B. subtilis EPSPS was found to be 88% and 100% resistant to inhibition by 1 mM glyphosate under these conditions for the subcloned (pMON21133) and PCR-derived (pMON21132) enzymes, respectively. The appkₘ(PEP) and the appKi(glyphosate) of the subcloned B. subtilis EPSPS (pMON21133) were determined as described above. The data were analyzed graphically by the same methods used for the 1A2 isolate, and the results obtained were comparable to those reported above for B. subtilis 1A2 culture.

Characterization of the EPSPS gene from Staphyloccus aureus

The kinetic properties of the S. aureus EPSPS expressed in E. coli were determined, including the specific activity, the appKₘ(PEP), and the appKᵢ(glyphosate). The S. aureus EPSPS gene has been previously described (O'C.onnell et al., 1993)

The strategy taken for the cloning of the S. aureus EPSPS was polymerase chain reaction (PCR), utilizing the known nucleotide sequence of the S. aureus aroA gene encoding EPSPS (O° C.onnell et al., 1993). The S. aureus culture (ATCC 35556) was fermented in an M2 facility in three 250 mL shake flasks containing 55 mL of TYE (tryptone 5g(L, yeast extract 3 9/L, pH 6.8). The three flasks were inoculated with 1.5 mL each of a suspension made from freeze dried ATCC 35556 S. aureus cells in 90 mL of PBS (phosphate-buffered saline) buffer. Flasks were incubated at 30° C. for 5 days while shaking at 250 rpm. The resulting cells were Iysed (boiled in TE [tris/EDTA] buffer for 8 minutes) and the DNA utilized for PCR reactions. The EPSPS gene was amplified using PCR and engineered into an E coli expression vector as follows:

(i) two oligonucleotides were synthesized which incorporated two restriction enzyme recognition sites (NcoI and SacI) to the sequences of the oligonucleotides:

| | |
|---|---|
| GGGGCCATGGTAAATGAACAAATCATTG | (SEQ ID NO:47) |
| GGGGGAGCTCATTATCCCTCATTTTGTAAAAGC | (SEQ ID NO:48) |

(ii) The purified, PCR-amplfied aroa gene from S. aureus was digested using NcoI and Sacd enzymes.

(ii) DNA of pMON 5723, which contains a pRecA bacterial promoter and Gene10 leader sequence (Olins et al., 1988) was digested NcoI and SacI and the 3.5 kb digestion product was purified.

(iv) The S. aureus PCR product and the NcoI/Sacl pMON 5723 fragment were ligated and transformed into E. coli JM101 competent cells.

(v) Two spectinomycin-resistant E. coli JM101 clones from above (SA#2 and SA#3) were purified and transformed into a competent aroa-E. coli strain, GB100

For complementation experiments SAGB#2 and SAGB#3 were utilized, which correspond to SA#2 and SA#3, respectively, transformed into E. coli GB100. In addition, E. coli GB100 (negative control) and pMON 9563 (wt petunia EPSPS, positive control) were tested for AroA complementation. The organisms were grown in minimal media plus and minus aromatic amino acids. Later analyses showed that the SA#2 and SA#3 clones were identical, and they were assigned the plasmid identifier pMON21139.

SAGB#2 in E. coli GB100 (pMON21139) was also grown in M9 minimal media and induced with nalidixic acid. A negative control, E. coli GB100, was grown under identical conditions except the media was supplemented with aromatic amino acids. The cells were harvested, washed with 0.9% NaCl, and frozen at −80° C., for extraction and EPSPS analysis.

The frozen pMON21139 E. coli GB100 cell pellet from above was extracted and assayed for EPSPS activity as previously described. EPSPS assays were performed using 1 mM phosphoenolpyruvate (PEP), 2 mM shikimate-3-phosphate (S3P), 0. 1 mM ammonium molybdate, 5 mM potassium fluoride, pH 7.0, 25° C. The total assay volume was 50 μL, which contained 10 μL of the undiluted desalted extract.

The results indicate that the two clones contain a functional aroA/EPSPS gene since they were able to grow in minimal media which contained no aromatic amino acids. As expected, the GB100 culture did not grow on minimal medium without aromatic amino acids (since no functional EPSPS is present), and the pMON9563 did confer growth in minimal media. These results demonstrated the successful cloning of a functional EPSPS gene from S. aureus. Both clones tested were identical, and the E. coli expression vector was designated pMON21139.

The plasmid pMON21139 in E. coli GB100 was grown in M9 minimal media and was induced with nalidixic acid to induce EPSPS expression driven from the RecA promoter. A desalted extract of the intracellular protein was analyzed for EPSPS activity, yielding an EPSPS specific activity of 0.005 μmol/min mg. Under these assay conditions, the S. aureus EPSPS activity was completely resistant to inhibition by 1 mM glyphosate. Previous analysis had shown that E. coli GB100 is devoid of EPSPS activity.

The appkₘ(PEP) of the S. aureus EPSPS was determined by measuring the reaction velocity of the enzyme kin crude bacterial extracts) at varying concentrations of PEP. The results were analyzed graphically using several standard kinetic plotting methods. Data analysis using the hyperbolic. Lineweaver-Burke, and Eadie-Hofstee methods yielded appkₘ(PEP) constants of 7.5, 4.8, and 4.0 μM. respectively. These three data treatments are in good agreement, and vield an average value for appK,(PEP) of 5 μM.

Further information of the glyphosate tolerance of S. aureus EPSPS was obtained by determining the reaction rates of the enzyme in the presence of several concentrations of glyphosate, at a PEP concentration of 2 μM. These results were compared to the calculated maximal velocity of the EPSPS, and making the assumption that glyphosate is a competitive inhibitor versus PEP for S. aureus EPSPS, as it is for all other characterized EPSPSs, an appKᵢ(glyphosate). was determined graphically. The appKᵢ(glyphosate) for S. aureus EPSPS estimated using this method was found to be 0.20 raK

The EPSPS from S. aureus was found to be glyphosate-tolerant, with an appKⱼ(glyphosate) of approximately 0.2 mM. In addition, the appkₘ(PEP) for the enzyme is approximately 5 μM, yielding a appKᵢ(glyphosate)/appkₘ(PEP) of 40.

Alternative Isolation Protocols for Other Class II EPSPS Structural Genes

A number of Class II genes have been isolated and described here. While the cloning of the gene from CP4 was difficult due to the low degree of similarity between the Class I and Class II enzymes and genes, the identification of the other genes was greatly facilitated by the use of this first gene as a probe. In the cloning of the LBAA EPSPS gene. the CP4 gene probe allowed the rapid identification of cosmid clones and the localization of the intact gene to a small restriction fragment and some of the CP4 sequencing primers were also used to sequence the LBAA (and PG2982) EPSPS gene(s). The CP4 gene probe was also used to confirm the PG2982 gene clone. The high degree of similarity of the Class II EPSPS genes may be used to identify and clone additional genes in much the same way that Class I EPSPS gene probes have been used to clone other Class I genes. An example of the latter was in the cloning of the A. thaliana EPSPS gene using the P. hybrida gene as a probe (Klee et al., 1987).

Glyphosate-tolerant EPSPS activity has been reported previously for EPSP synthases from a number of sources. These enzymes have not been characterized to any extent in most cases. The use of Class I and Class II EPSPS gene probes or antibody probes provide a rapid means of initially screening for the nature of the EPSPS and provide tools for the rapid cloning and characterization of the genes for such enzymes.

Two of the three genes described were isolated from bacteria that were isolated from a glYphosate treatment facility (Strains CP4 and LBAA). The third (PG2982) was from a bacterium that had been isolated from a culture collection strain. This latter isolation confirms that exposure to glyphosate is not a prerequisite for the isolation of high glyphosate-tolerant EPSPS enzymes and that the screening of collections of bacteria could yield additional isolates. It is possible to enrich for glyphosate degrading or glyphosate resistant microbial populations (Quinn et al., 1988; Talbot et al., 1984) in cases where it was felt that enrichment for such microorganisms would enhance the isolation frequency of Class II EPSPS microorganisms. Additional bacteria containing class II EPSPS gene have also been identified. A bacterium called C12, isolated from the same treatment column beads as CP4 (see above) but in a medium in which glyphosate was supplied as both the carbon and phosphorus source, was shown by Southern analysis to hybridize with a probe consisting of the CP4 EPSPS coding sequence. This result, in conjunction with that for strain LBAA, suggests that this enrichment method facilitates the identification of Class II EPSPS isolates. New bacterial isolates containing Class II EPSPS genes have also been identified from environments other than glyphosate waste treatment facilities. An inoculum was prepared by extracting soil (from a recently harvested soybean field in Jerseyville, Ill.) and a population of bacteria selected by growth at 280C in Dworkin-Foster medium containing glyphosate at 10 mM as a source of carbon (and with cycloheximide at 100 ,g/ml to prevent the growth of fungi). Upon plating on L-agar media, five colony types were identified. Chromosomal DNA was prepared from 2ml L-broth cultures of these isolates and the presence of a Class II EPSPS gene was probed using a the CP4 EPSPS coding sequence probe by Southern analysis under stringent hybridization and washing conditions. One of the soil isolates, S2, was positive by this screen.

Class II EPSPS enzymes are identifiable by an elevated Kᵢ for glyphosate and thus the genes for these will impart a glyphosate tolerance phenotype in heterologous hosts. Expression of the gene from recombinant plasmids or phage may be achieved through the use of a variety of expression promoters and irclude the T7 promoter and polymerase. The T7 promoter and polymerase system has been shown to work in a wide range of bacterial (and mammalian) hosts and offers the advantage of expression of many proteins that may be present on large cloned fragments. Tolerance to growth on glyphosate may be shown on minimal growth media. In some cases, other genes or conditions that may give glyphosate tolerance have been observed, including over expression of beta-lactamase, the igrA gene (Fitzgibbon and Braymer, 1990), or the gene for glyphosate oxidoreductase (PCT Pub. No. W092/00377). These are easily distinguished from Class II EPSPS by the absence of EPSPS enzyme activity.

The EPSPS protein is expressed from the aroA gene (also called aroE in some genera, for example, in Bacillus) and mutants in this gene have been produced in a wide variety of bacteria. Determining the identity of the donor organism (bacterium) aids in the isolation of Class II EPSPS gene—such identification may be accomplished by standard microbiological methods and could include Gram stain reaction, growth, color of culture, and gas or acid production on different substrates, gas chromatography analysis of methylesters of the fatty acids in the membranes of the microorganism, and determination of the GC% of the genome. The identity of the donor provides information that may be used to more easily isolate the EPSPS gene. An AroA-host more closely related to the donor organism could be employed to clone the EPSPS gene by complementation but this is not essential since complementation of the E. coli AroA mutant by the CP4 EPSPS gene was observed. In addition. the information on the GC content the genome may be used in chooosing nucleotide probes -donor sources with high GC% would preferably use the CP4 EPSPS gene or sequences as probes and those donors with low GC would preferably employ those from Bacillus subtilis , for example.

Relationships between different EPSPS genes

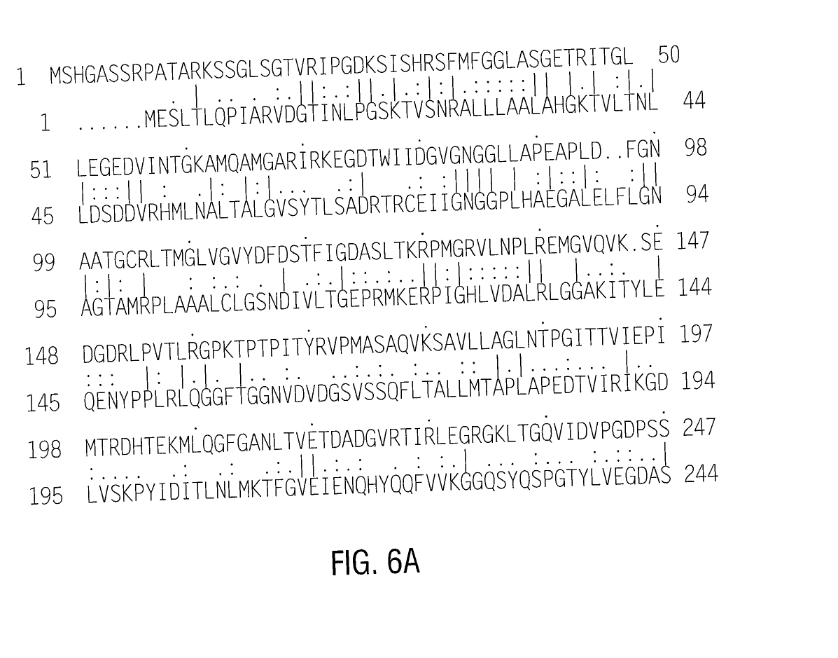

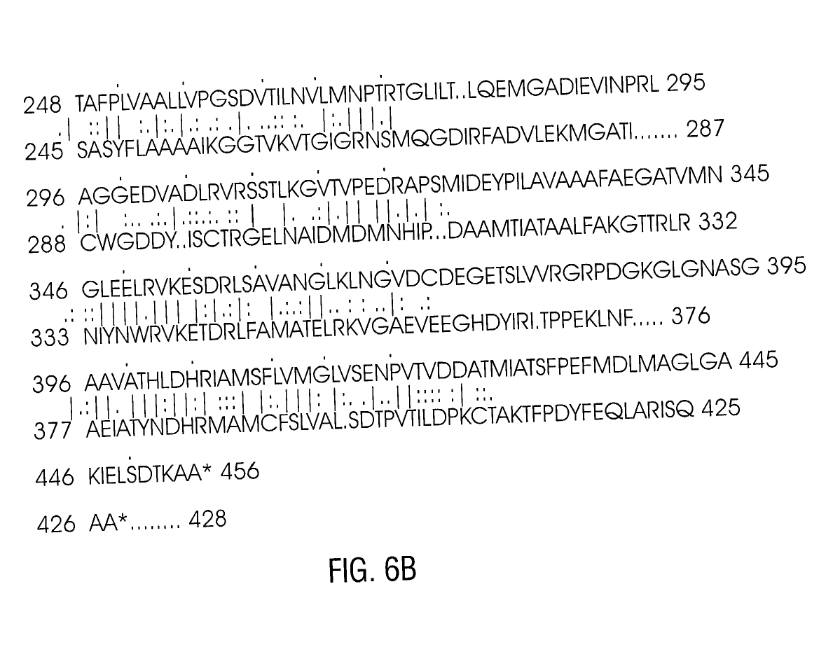

The deduced amino acid sequences of a number of Class I and the Class II EPSPS enzymes were compared using the Bestfit computer program provided in the UWGCG package (Devereux et al. 1984). The degree of similarity and identity as determined using this program is reported. The degree of similarity/identity determined within Class I and Class II protein sequences is remarkably high, for instance, comparing E. coli with S. typhimurium (similarity/identity =93%/c/88%) and even comparing E. coli with a plant EPSPS (Petunia hybrida; 72%1/c/55%). These data are shown in Table W. The comparison of sequences between Class I and Class II, however, shows a much lower degree of relatedness between the Classes (similarity/identity=50-53%/c23-30%). The display of the Bestfit analysis for the E. coli (SEQ ID NO:8) and CP4 (SEQ ID NO:3) sequences shows the positions of the conserved residues and is presented in FIG. 6. Previous analyses of EPSPS sequences had noted the high degree of conservation of sequences of the enzymes and the almost invariance of sequences in two regions—the “20-35” and “95-107” regions (Gasser et al., 1988; numbered according to the Petunia EPSPS sequence)—and these regions are less conserved in the case of CP4 and LBAA when compared to Class I bacterial and plant EPSPS sequences (see FIG. 6 for a comparison of the E. coli and CP4 EPSPS sequences with the E. coli sequence appearing as the top sequence in the Figure). The corresponding sequences in the CP4 Class II EPSPS are:

| | |
|---|---|
| PGDKSISHRSFMFGGL | (SEQ ID NO:32) and |
| LDFGNAATGCRLT | (SEQ ID NO:33). |

These comparisons show that the overall relatedness of Class I and Class II is EPSPS proteins is low and that sequences in putative conserved regions have also diverged considerably.

In the CP4 EPSPS an alanine residue is present at the “glycine101” position. The replacement of the conserved glycine (from the “95-107” region) by an alanine results in an elevated Kᵢ for glyphosate and in an elevation in the Kₘ for PEP in Class I EPSPS. In the case of the CP4 EPSPS, which contains an alanine at this position, the Kₘ for PEP is in the low range, indicating that the Class II enzymes differ in many aspects from the EPSPS enzymes heretofore characterized.

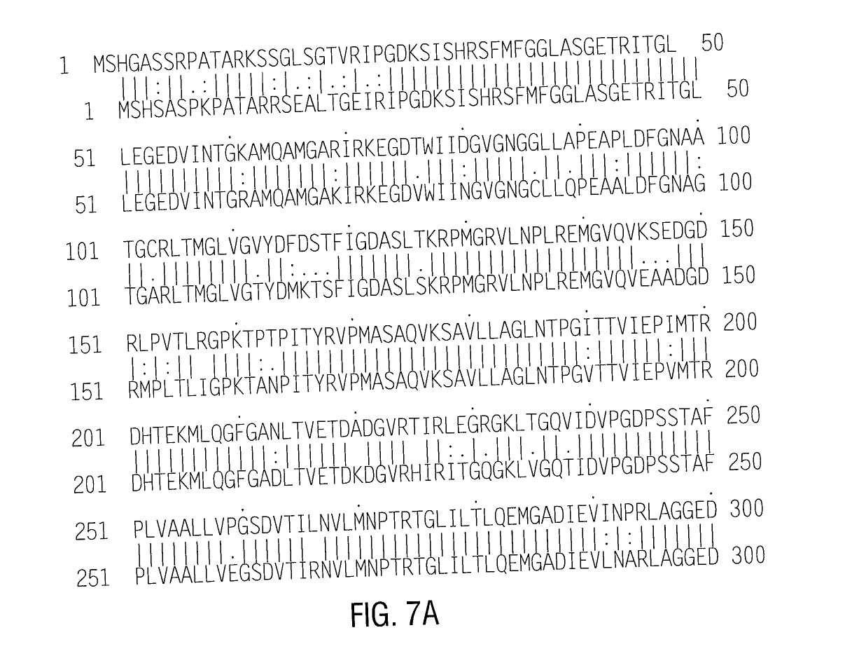

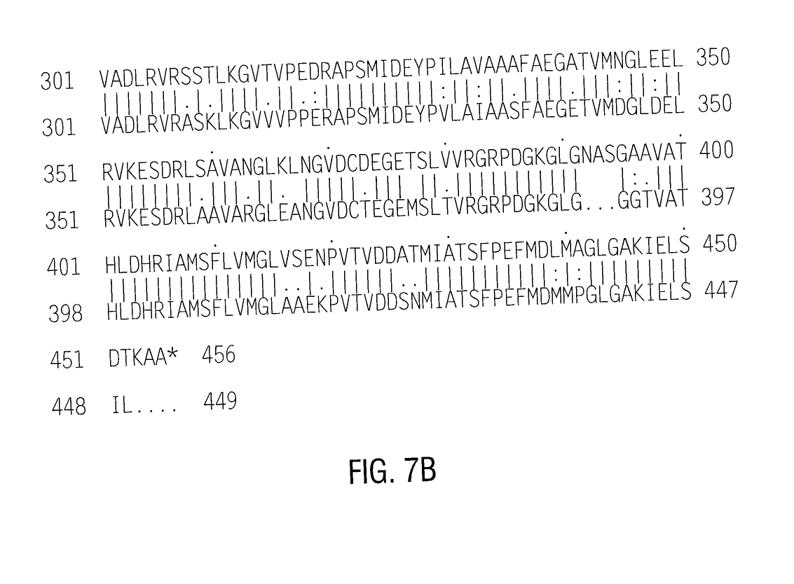

Within the Class II isolates, the degree of similarity/identity is as high as that noted for that within Class I (Table IVA). FIG. 7 displays the Bestfit computer program alignment of the CP4 (SEQ ID NO:3) and LBAA (SEQ ID NO:5) EPSPS deduced amino acid sequences with the CP4 sequence appealing as the top sequence in the Figure. The symbols used in FIGS. 6 and 7 are the standard symbols used in the Bestfit computer program to designate degrees of similarity and identity.

TABLE IVA^{1,2}
Comparison of relatedness of EPSPS protein sequences
Comparison between Class I and Class II EPSPS protein sequences

| | similarity | identity |
|---|---|---|
| S. cerevisiae vs. CP4 | 54 | 30 |
| A. nidulans vs. CP4 | 50 | 25 |
| B. napus vs. CP4 | 47 | 22 |
| A. thaliana vs. CP4 | 48 | 22 |
| N. tabacum vs. CP4 | 50 | 24 |
| L. esculentum vs. CP4 | 50 | 24 |
| P. hybrida vs. CP4 | 50 | 23 |
| Z. mays vs. CP4 | 48 | 24 |
| S. gallinarum vs. CP4 | 51 | 25 |
| S. typhimurium vs. CP4 | 51 | 25 |
| S. typhi vs. CP4 | 51 | 25 |
| K. pneumoniae vs. CP4 | 56 | 28 |
| Y. enterocolitica vs. CP4 | 53 | 25 |
| H. influenzae vs. CP4 | 53 | 27 |
| P. multocida vs. CP4 | 55 | 30 |
| A. salmonicida vs. CP4 | 53 | 23 |
| B. pertussis vs. CP4 | 53 | 27 |
| E. coli vs. CP4 | 52 | 26 |
| E. coli vs. LBAA | 52 | 26 |
| E. coli vs. B. subtilis | 55 | 29 |
| E. coli vs. D. nodosus | 55 | 32 |
| E. coli vs. S. aureus | 55 | 29 |
| E. coli vs. Synechocystis sp. PCC6803 | 53 | 30 |

Comparison between Class I EPSPS protein sequences

| | | |
|---|---|---|
| E. coli vs. S. typhimurium | 93 | 88 |
| P. hybrida vs. E. coli | 72 | 55 |

Comparison between Class II EPSPS protein sequences

| | | |
|---|---|---|
| D. nodosus vs. CP4 | 62 | 43 |
| LBAA vs. CP4 | 90 | 83 |
| PG2892 vs. CP4 | 90 | 83 |
| S. aureus vs. CP4 | 58 | 34 |
| B. subtilis vs. CP4 | 59 | 41 |
| Synechocystis sp. PCC6803 vs. CP4 | 62 | 45 |

¹The EPSPS sequences compared here were obtained from the following references: E. coil, Rogers et al., 1983; S. typhimurium, Stalker et al., 1985; Petunia hybrida, Shah et al., 1986; B. pertussis, Maskell et al., 1988: S. cerevisiae, Duncan et al., 1987, Synechocystis sp. PCC6803, Dalla Chiesa et al., 1994 and D. nodosus, Alm et al., 1994.
²“GAP” Program. Genetics Computer Group, (1991), Program Manual for the GCG Package, Version 7, April 1991, 575 Science Drive, Madison, Wisconsin, USA 53711

TABLE IVB
Location of Conserved Sequences in
Class II EPSP Synthases

| Source | Seq. 1¹ | Seq. 2² | Seq. 3³ | Seq. 4⁴ |
|---|---|---|---|---|
| CP4 | | | | |
| start | 200 | 26 | 173 | 271 |
| end | 204 | 29 | 177 | 274 |
| LBAA | | | | |
| start | 200 | 26 | 173 | 271 |
| end | 204 | 29 | 177 | 274 |
| PG2982 | | | | |
| start | 200 | 26 | 173 | 273 |
| end | 204 | 29 | 177 | 276 |
| B. subtilis | | | | |
| start | 190 | 17 | 164 | 257 |
| end | 194 | 20 | 168 | 260 |
| S. aureus | | | | |
| start | 193 | 21 | 166 | 261 |
| end | 197 | 24 | 170 | 264 |
| Synechocystis sp. PCC6803 | | | | |
| start | 210 | 34 | 183 | 278 |
| end | 214 | 38 | 187 | 281 |
| D. nodosus | | | | |
| start | 195 | 22 | 168 | 261 |
| end | 199 | 25 | 172 | 264 |
| min. start | 190 | 17 | 164 | 257 |
| max. end | 214 | 38 | 187 | 281 |

¹-R-X₁-H-X₂-E- (SEQ ID NO: 37)
²-G-D-K-X₃- (SEQ ID NO: 38)
³—S-A-Q-X₄-K— (SEQ ID NO: 39)
⁴—N-X₅-T-R- (SEQ ID NO: 40)

The domains of EPSP synthase sequence identified in this application were determined to be those important for maintenance of glyphosate resistance and productive binding of PEP. The information used in indentifying these domains included sequence alignments of numerous glyphosate-sensitive EPSPS molecules and the three-dimensional x-ray structures of E. coli EPSPS (Stallings, et al. 1991) and CP4 EPSPS. The structures are representative of a glyphosate-sensitive (i.e., Class I) enzyme, and a naturally-occuring glyphosate-tolerant (i.e., Class II) enzyme of the present invention. These exemplary molecules were superposed three-dimensionally and the results displayed on a computer graphics terminal. Inspection of the display allowed for structure-based fine-tuning of the sequence alignments of glyphosate-sensitive and glyphosate-resistant EPSPS molecules. The new sequence alignments were examined to determine differences between Class I and Class II EPSPS enzymes. Seven regions were identified and these regions were located in the x-ray structure of CP4 EPSPS which also contained a bound analog of the intermediate which forms catalytically between PEP and S3P.

The structure of the CP4 EPSPS with the bound intermediate analog was displayed on a computer graphics terminal and the seven sequence segments were examined. Important residues for glyphosate binding were identified as well as those residues which stabilized the conformations of those important residues: adjoining residues were considered necessary for maintenance of correct three-dimensional structural motifs in the context of glyphosate-sensitive EPSPS molecules. Three of the seven domains were determined not to be important for glyphosate tolerance and maintainance of productive PEP binding. The following four primary domains were determined to be characteristic of Class II EPSPS enzymes of the present invention:

R—Xi-H—X₂—E (SEQ ID NO:37), in which

X₁ is an uncharged polar or acidic amino acid,

X₂ is serine or threonine,

The Arginine (R) reside at position 1 is important because the positive charge of its guanidium group destabilizes the binding of glyphosate. The Histidine (H) residue at position 3 stabilizes the Arginine (R) residue at position 4 of SEQ ID NO:40. The Glutamic Acid (E) residue at position 5 stabilizes the Lysine (K) residue at position 5 of SEQ ID NO:39.

—G—D—I—X₃ (SEQ ID NO:38), in which

X₃ is serme or threonie,

The Aspartic acid (D) residue at position 2 stabilizes the Arginine (R) residue at position 4 of SEQ ID NO:40. The Lysine (K) residue at position 3 is important because for productive PEP binding.

—S—A—Q—X₄—K (SEQ ID NO:39), in which

X₄ is any amino acid,

The Alanine (A) residue at position 2 stabilizes the Arginine (R) residue at position 1 of SEQ ID NO:37. The Serine (S) residue at position 1 and the Glutamine (Q) residue at position 3 are important for productive S3P binding.

—N-X₅—T—R (SEQ ID NO:40) in which

X₅ is any amino acid,

The Asparagine (N) residue at position 1 and the Threonine (T) residue at position 3 stabilize residue X₁ at position 2 of SEQ ID NO:37. The Arginine (R) residue at position 4 is important because the positive charge of its guanidium group destabilizes the binding of glyphosate.

Since the above sequences are only representative of the Class II EPSPSs which would be included within the generic structure of this group of EPSP synthases, the above sequences may be found within a subject EPSP synthase molecule within slightly more expanded regions. It is believed that the above-described conserved sequences would likely be found in the following regions of the mature EPSP synthases molecule:

—R—X₁—H—X₂—E—(SEQ ID NO:37) located between amino acids 175 and 230 of the mature EPSP synthase sequence;

—G—D—K—X₃—(SEQ ID NO:38) located between amino acids 5 and 55 of the mature EPSP synthase sequence;

—S—A—Q—X₄—K—(SEQ ID NO:39) located between amino acids 150 and 200 of the mature EPSP synthase sequence; and

—N—X₅—T—R—(SEQ ID NO:40) located between amino acids 245 and 295 of the mature EPSPS synthase sequence.

One difference that may be noted between the deduced amino acid sequences of the CP4 and LBAA EPSPS proteins is at position 100 where an Alanine is found in the case of the CP4 enzyme and a Glycine is found in the case of the LBAA enzyme. In the Class I EPSPS enzymes a Glycine is usually found in the equivalent position, i.e Glycine96 in E. coli and K pneumoniae and Glycine101 in Petunia. In the case of these three enzymes it has been reported that converting that Glycine to an Alanine results in an elevation of the appKᵢfor glyphosate and a concomitant elevation in the appkₘ for PEP (Kishore et al., 1986; Kᵢ shore and Shah, 1988; Sost and Amrhein, 1990), which, as discussed above, makes the enzyme less efficient especially under conditions of lower PEP concentrations. The Glycine100 of the LBAA EPSPS was converted to an Alanine and both the appkₘ for PEP and the appKᵢ for glyphosate were determined for the variant. The Glycine100Alanine change was introduced by mutagenesis using the following primer:

| | |
|---|---|
| CGGCAATGCCGCCACCGGCGCGCGCG | (SEQ ID NO:34) |

and both the wild type and variant genes were expressed in E. coli in a RecA promoter expression vector (pMON17201 and pMON17264, respectively) and the appkₘ's and appKᵢ's determined in crude lysates. The data indicate that the appKᵢ(glyphosate) for the G1OOA variant is elevated about 16-fold (Table V). This result is in agreement with the observation of the importance of this G-A change in raising the appKᵢ(glyphosate) in the Class I EPSPS enzymes. However, in contrast to the results in the Class I G-A variants, the appkₘ(PEP) in the Class II (LBAA) G-A variant is unaltered. This provides yet another distinction between the Class II and Class I EPSPS enzymes.

TABLE V

| | appKm (PEP) | appKi (glyphosate) |
|---|---|---|
| Lysate prepared from: | | |
| E. coli/pMON17201 (wild type) | 5.3 μM | 28 μM* |
| E. coli/pMON17264 | 5.5 μM | 459 μM# |
| (G100A variant) | | |

@ range of PEP: 2-40 μM
* range of glyphosate: 0-310 μM; # range of glyphosate: 0-5000 μM.
The LBAA G100A variant, by virtue of its superior kinetic properties, should be capable of imparting improved in planta glyphosate tolerance.

Modification and Resynthesis of the Agrobacterium sp. strain CP4 EPSPS Gene Sequence

The EPSPS gene from Agrobacterium sp. strain CP4 contains sequences that could be inimical to high expression of the gene in plants. These sequences include potential polyadenvlation sites that are often and A+T rich, a higher G+C% than that frequently found in plant genes (63% versus 50%), concentrated stretches of G and C residues, and codons that are not used frequently in plant genes The high G+C% in the CP4 EPSPS gene has a number of potential consequences including the following: a higher usage of G or C than that found in plant genes in the third position in codons, and the potential to form strong hair-pin structures that may affect expression or stability of the RNA. The reduction in the G+C content of the CP4 EPSPS gene, the disruption of stretches of G's and C's, the elimination of potential polyadenylation sequences, and improvements in the codon usage to that used more frequently in plant genes, could result in higher expression of the CP4 EPSPS gene in plants.

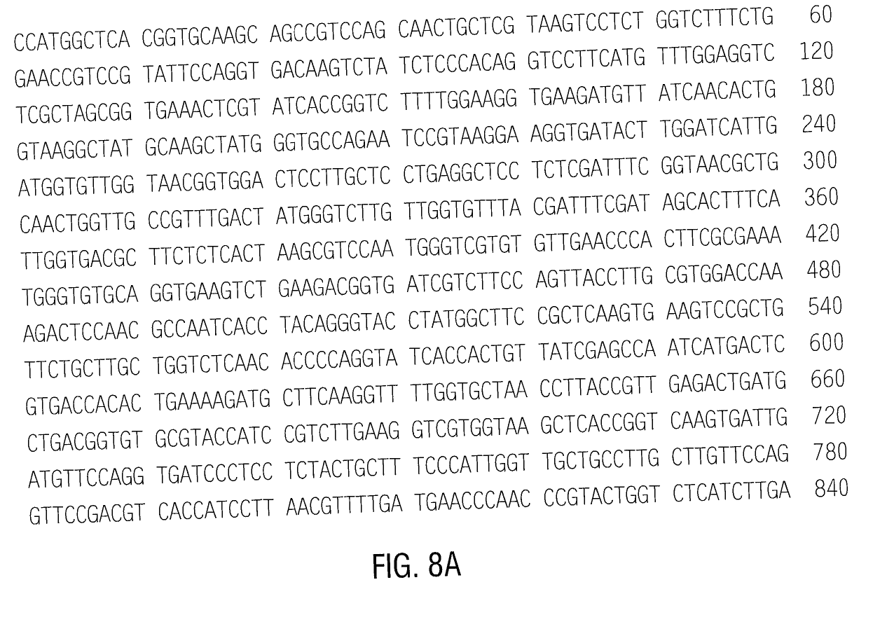

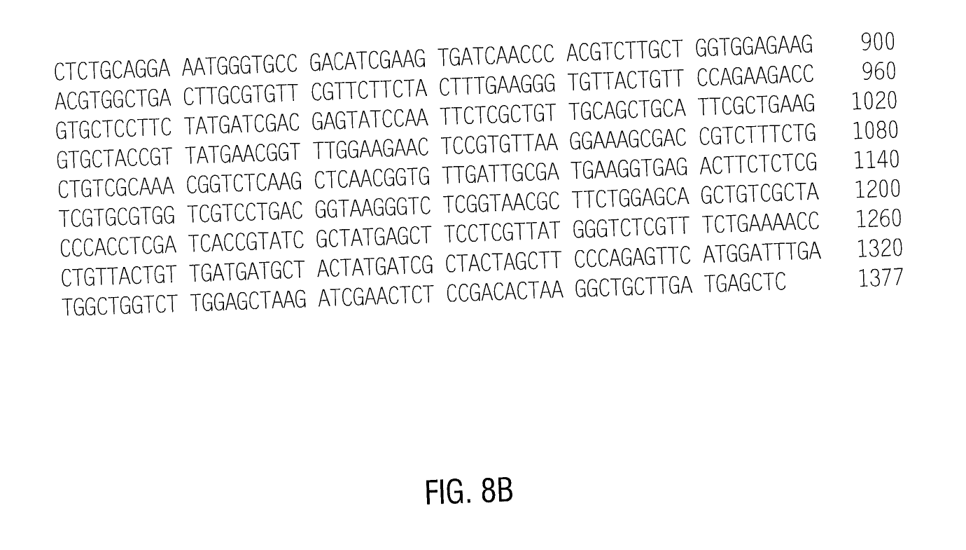

A synthetic CP4 gene was designed to change as completely as possible those inimical sequences discussed above. In summary, the gene sequence was redesigned to eliminate as much as possible the following sequences or sequence features (while avoiding the introduction of unnecessary restriction sites): stretches of G's and C's of 5 or greater; and A+T rich regions (predominantly) that could function as polyadenylation sites or potential RNA destabilization region The sequence of this gene is shown in FIG. 8 (SEQ ID NO:9). This coding sequence was expressed in E. coli from the RecA promoter and assayed for EPSPS activity and compared with that from the native CP4 EPSPS gene. The apparent Kₘ for PEP for the native and synthetic genes was 11.8 and 12.7, respectively, indicating that the enzyme expressed from the synthetic gene was unaltered. The N-terminus of the coding sequence was mutagenized to place an SphI site at the ATG to permit the construction of the CTP2-CP4 synthetic fusion for chloroplast import. The following primer was used to accomplish this mutagenesis:

| | |
|---|---|
| GGACGGCTGCTTGCACCGTGAAGCATGCTTAAGCTTGGCGTAATCATGG | (SEQ ID NO:35). |

Eression of Cloroplast Directed CP4 EPSPS

The glyphosate target in plants, the 5-enolpyruvyl-shikimate-3-phosphate synthase (EPSPS) enzyme. is located in the chloroplast. Many chloroplast-localized proteins, including EPSPS. are expressed from nuclear genes as precursors and are targeted to the chloroplast by a chloroplast transit peptide (CTP) that is removed during the import steps. Examples of other such chloroplast proteins include the small subunit (SSU) of Ribulose-1,5-bisphosphate carboxylase (RUBISCO), Ferredoxin, Ferredoxin oxidoreductase, the Light-harvesting-complex protein I and protein II, and Thioredoxin F. It has been demonstrated in vivo and in vitro that non-chloroplast proteins may be targeted to the chloroplast by use of protein fusions with a CTP and that a CTP sequence is sufficient to target a protein to the chloroplast.

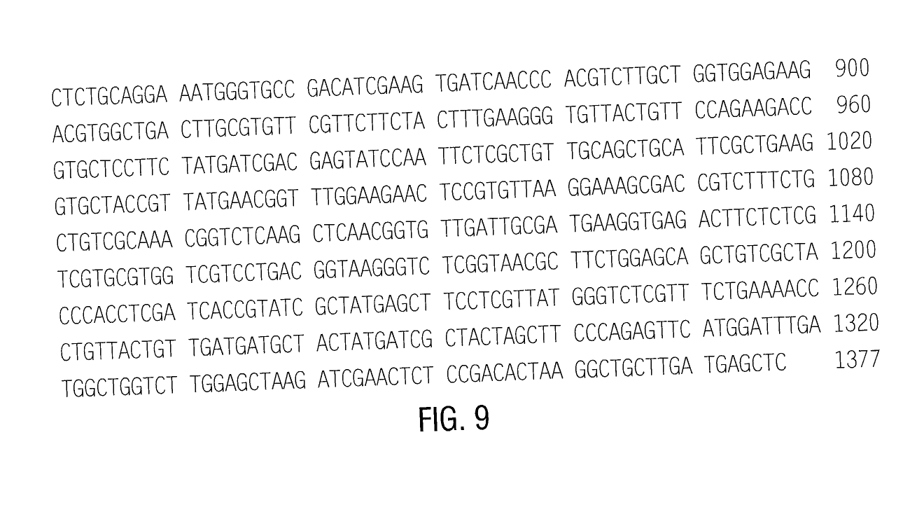

A CTP-CP4 EPSPS fusion was constructed between the Arabidopsis thaliana EPSPS CTP (Klee et al., 1987) and the CP4 EPSPS coding sequences. The Arabidopsis CTP was engineered by site-directed mutagenesis to place a SphI restriction site at the CTP processing site. This mutagenesis replaced the Glu-Lys at this location with Cys-Met. The sequence of this CTP, designated as CTP2 (SEQ ID NO:10), is shown in FIG. 9. The N-terminus of the CP4 EPSPS gene was modified to place a SphI site that spans the Met codon. The second codon was converted to one for leucine in this step also. This change had no apparent effect on the in vivo activity of CP4 EPSPS in E. coli as judged by rate of complementation of the aroA allele. This modified N-terminus was then combined with the Sacl C-terminus and cloned downstream of the CTP2 sequences. The CTP2-CP4 EPSPS fusion was cloned into pBlueScript KS(+). This vector may be transcribed in vitro using the T7 polymerase and the RNA translated with ³⁵S-Methionine to provide material that may be evaluated for import into chloroplasts isolated from Lactuca sativa using the methods described hereinafter tdella-Cioppa et al., 1986, 1987). This template was transcribed in vitro using T7 polymerase and the ³⁵S-methionine-labeled CTP2-CP4 EPSPS material was shown to import into chloroplasts with an efficiency comparable to that for the control Petunia EPSPS (control=³⁵S labeled PreEPSPS [pMON6140: della-Cioppa et al. 1986]).

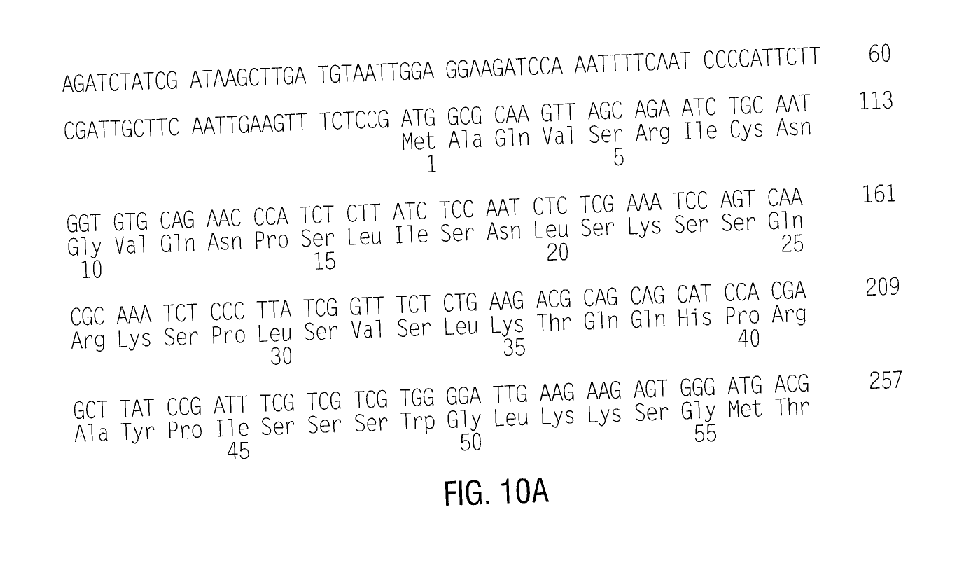

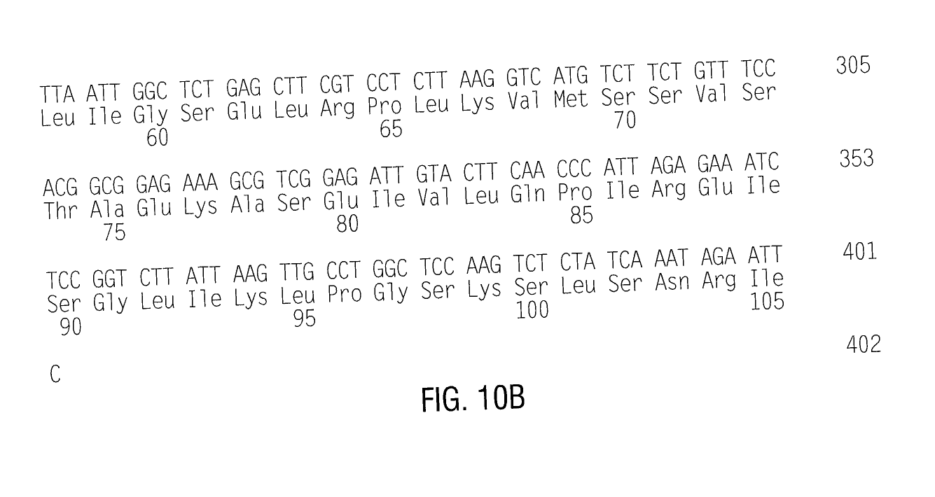

In another example the Arabidopsis EPSPS CTP? designated as CTP3, was fused to the CP4 EPSPS through an EcoRI site. The sequence of this CTP3 (SEQ ID NO:12) is shown in FIG. 10. An EcoRI site was introduced into the Arabidopsis EPSPS mature region around amino acid 27, replacing the sequence -Arg-Ala-Leu-Leu-with -Arg-Ee-Leu-Leu-in the process. The primer of the following sequence was used to modify the N-terminus of the CP4 EPSPS gene to add an EcoRI site to effect the fusion to the

| | |
|---|---|
| CTP3:GGAAGACGCCCAGAATTCACGGTGCAAGCAGCCGG | (SEQ ID NO:36) (the EcaRI site is underlined. |

This CTP3-CP4 EPSPS fusion was also cloned into the pBlueScript vector and the T7 expressed fusion was found to also import into chloroplasts with an efficiency comparable to that for the control Petunia EPSPS (pMON6140).

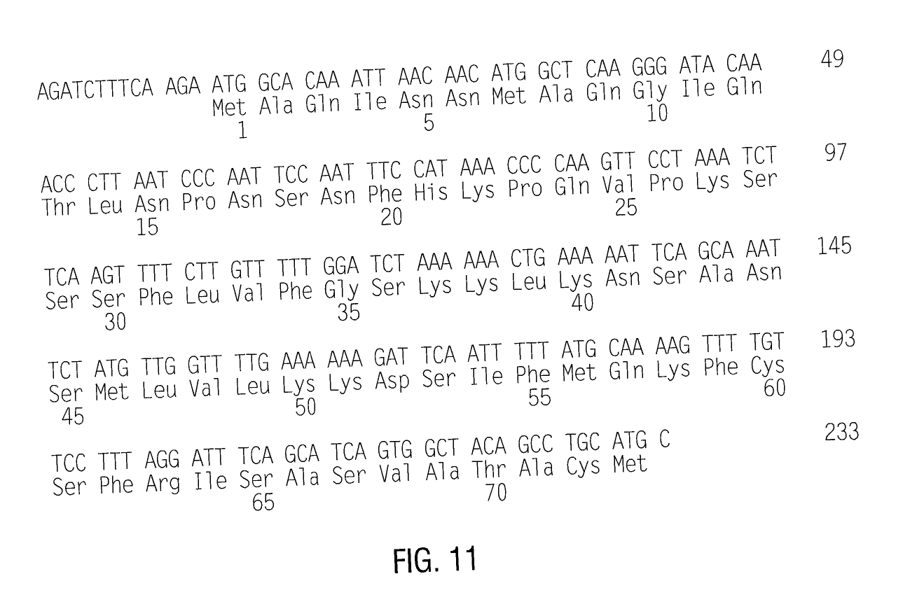

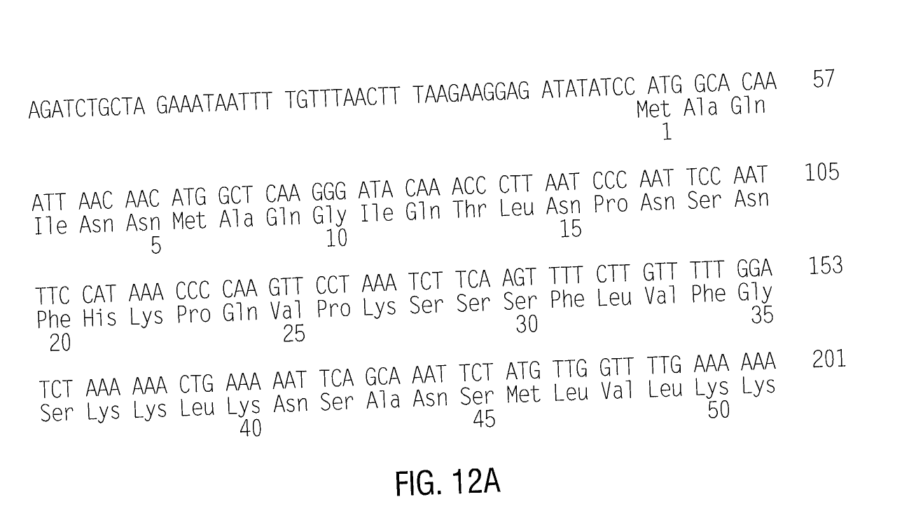

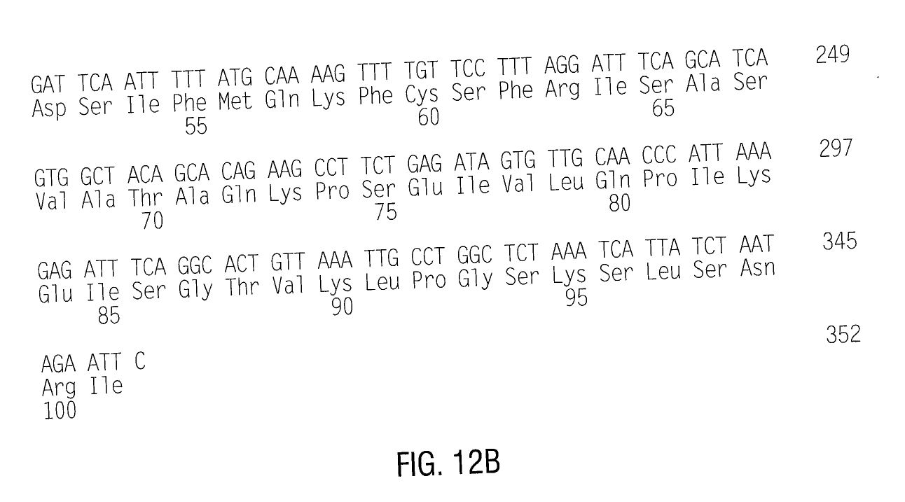

A related series of CTPs, designated as CTP4 (SphI) and CTP5 (EcoRI), based on the Petunia EPSPS CTP and gene were also fused to the SphI-and EcoRI-modified CP4 EPSPS gene sequences. The SphI site was added by site-directed mutagenesis to place this restriction site (and change the amino acid sequence to -Cys-Met-) at the chloroplast processing site. All of the CTP-CP4 EPSPS fusions were shown to import into chloroplasts with approximately equal efficiency. The CTP4 (SEQ ID NO:14) and CTP5 (SEQ ID NO:16) sequences are shown in FIGS. 11 and 12.

A CTP2-LBAA EPSPS fusion was also constructed following the modification of the N-terminus of the LBAA EPSPS gene by the addition of a SphI site. This fusion was also found to be imported efficiently into chloroplasts.

By similar approaches, the CTP2-CP4 EPSPS and the CTP4-CP4 EPSPS fusion have also been shown to import efficiently into chloroplasts prepared from the leaf sheaths of corn. These results indicate that these CTP-CP4 fusions could also provide useful genes to impart glyphosate tolerance in monocot species.

The use of CTP2 or CTP4 is preferred because these transit peptide constructions yield mature EPSPS enzymes upon import into the chloroplat which are closer in composition to the native EPSPSs not containing a transit peptide signal. Those skilled in the art will recognize that various chimeric constructs can be made which utilize the functionality of a particular CTP to import a Class II EPSPS enzyme into the plant cell chloroplast. The chloroplast import of the Class II EPSPS can be determined using the following assay.

Chloroplast Uptake Assay

Intact chloroplasts are isolated from lettuce (Latuca, sativa, var. longifolia) by centrifugation in Percoll/ficoll gradients as modified from Bartlett et al., (1982). The final pellet of intact chloroplasts is suspended in 0.5 ml of sterile 330 mM sorbitol in 50 mM Hepes-KOH, pH 7.7, assayed for chlorophyll (Arnon, 1949), and adjusted to the final chlorophyll concentration of 4 mg/ml (using sorbitol/Hepes). The yield of intact chloroplasts from a single head of lettuce is 3-6mg chlorophyll.

A typical 300 μl uptake experiment contained 5 mM ATP, 8.3 mM unlabeled methionine, 322 mM sorbitol, 58.3 mM Hepes-KOH (pH 8.0), 50 μl reticulocyte lysate translation products, and intact chloroplasts from L. sativa (200 μg chlorophyll). The uptake mixture is gently rocked at room temperature (in 10×75 mm glass tubes) directly in front of a fiber optic illuminator set at maximum light intensity (150 Watt bulb). Aliquot samples of the uptake mix (about 50 μl) are removed at various times and fractionated over 100 μl silicone-oil gradients (in 150μl polyethylene tubes) by centrifugation at 11,000 X g for 30 seconds. Under these conditions, the intact chloroplasts form a pellet under the silicone-oil layer and the incubation medium (containing the reticulocyte lysate floats on the surface. After centrifugation, the silicone-oil gradients are immediately frozen in dry ice. The chloroplast pellet is then resuspended in 50-100 41 of lysis buffer (10mM Hepes-KOH pH 7.5, 1 mM PMSF, 1 mM benzamidine, 5 mM e-amino-n-caproic acid, and 30 μg/ml aprotinin) and centrifuged at 15,000 X g for 20 minutes to pellet the thylakoid membranes. The clear supernatant (stromal proteins) from this spin, and an aliquot of the reticulocyte lysate incubation medium from each uptake experiment, are mixed with an equal volume of 2X SDS-PAGE sample buffer for electrophoresis (Laemmli, 1970).

SDS-PAGE is carried out according to Laemmli (1970) in 3-17% (w/v) acrylamide slab gels (60 mm X 1.5 mm) with 3% (w/v) acrylamide stacking gels (5 mm×1.5 mm). The gel is fixed for 20-30 min in a solution with 40% methanol and 10% acetic acid. Then, the gel is soaked in EN³HANCE™ (DuPont) for 20-30 minutes, followed by drying the gel on a gel dryer. The gel is imaged by autoradiography, using an intensifying screen and an overnight exposure to determine whether the CP4 EPSPS is imported into the isolated chloroplasts.

Plant Transformation

Plants which can be made glyphosate-tolerant by practice of the present invention include, but are not limited to, soybean, cotton, corn, canola, oil seed rape, flax. sugarbeet, sunflower, potato, tobacco. tomato, wheat, rice, alfalfa and lettuce as well as various tree, nut and vine species.

A double-stranded DNA molecule of the present invention (“chimeric gene”) can be inserted into the genome of a plant by any suitable method. Suitable plant transformation vectors include those derived from a Ti plasmid of Agrobacterium tumefaciens, as well as those disclosed, e.g., by Herrera-Estrella (1983), Bevan (1984), Kᵢ ee (1985) and EPO publication 120,516 (Schilperoort et aI.). In addition to plant transformation vectors derived from the Ti or root-inducing R₁ plasmids of Agrobacterium. alternative methods can be used to insert the DNA constructs of this invention into plant cells. Such methods may Involye. for example. the use of liposomes, electroporation. chemicals that increase free DNA uptake, free DNA delivery via microprojectile bombardment, and transformation using viruses or pollen.

Class II EPSPS Plant transformation vectors

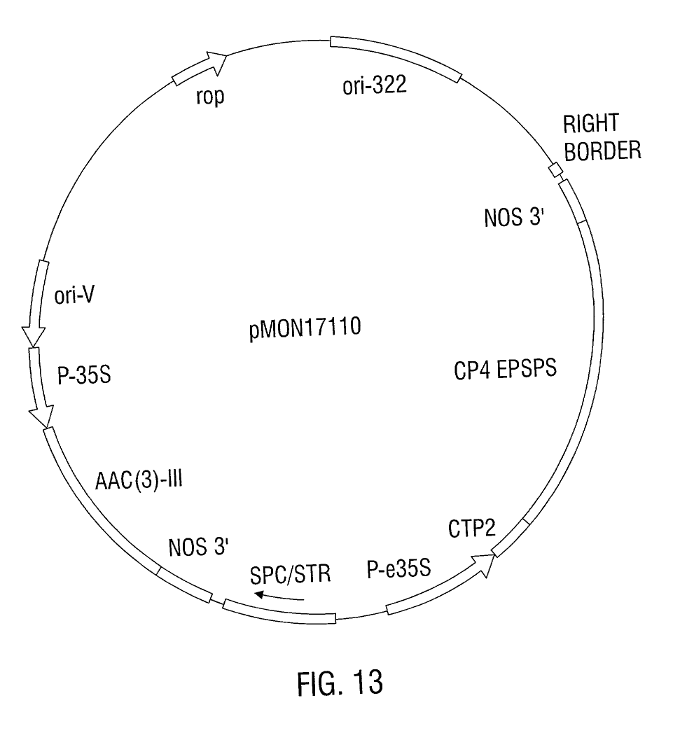

Class II EPSPS DNA sequences may be engineered into vectors capable of transforming plants by using known techniques The following description is meant to be illustrative and not to be read in a limiting sense. One of ordinary skill in the art would know that other plasmids, vectors, markers, promoters, etc. would be used with suitable results. The CTP2-CP4 EPSPS fusion was cloned as a BglII-EcoRI fragment into the plant vector pMON979 (described below) to form pMON17110, a map of which is presented in FIG. 13. In this vector the CP4 gene is expressed from the enhanced CaMV³⁵S promoter (E³⁵S; Kay et al. 1987). A FMV³⁵S promoter construct (pMON17116) was completed in the following way: The SalI—NotI and the NotI-BglII fragments from pMON979 containing the Spc(AAC(3)-III/oriV and the pBR322/Right Border/NOS 3′/CP4 EPSPS gene segment from pMON17110 were ligated with the XhoI-BglII FMV³⁵S promoter fragment from pMON981. These vectors were introduced into tobacco, cotton and canola.

A series of vectors was also completed in the vector pMON977 in which the CP4 EPSPS gene, the CTP2-CP4 EPSPS fusion, and the CTP3-CP4 fusion were cloned as BglII-SacI fragments to form pMON17124, pMON17119, and pMON17120, respectively. These plasmids were introduced into tobacco. A pMON977 derivative containing the CTP2-LBAA EPSPS gene was also completed (pMON17206) and introduced into tobacco.

The pMON979 plant transformation/expression vector was derived from pMON886 (described below by replacing the neomycin phosphotransferase typeII (KAN) gene in pMON886 with the 0.89 kb fragment containing the bacterial gentamicin-3—N-acetvltransferase type III (AAC(3)-III) gene (Hayford et al., 1988). The chimeric P-³⁵S/AA(3)-III/NOS 3′ gene encodes gentamicin resistance which permits selection of transformed plant cells. pMON979 also contains a 0.95 kb expression cassette consisting of the enhanced CaMV ³⁵S promoter (Kay et al., 1987), several unique restriction sites, and the NOS 3′ end (P-En-CaMV³⁵S/NOS 3′). The rest of the pMON979 DNA segments are exactly the same as in pMON886.

Plasmid pM ON886 is made up of the following segments of DNA The first is a 0.93 kb Aval to engineered-EcoRV fragment isolated from transposon Tn7 that encodes bacterial spectinomycin/streptomycin resistance (Spc/Str), which is a determinant for selection in E. coli and Agrobacterium tumefaciens. This is joined to the 1.61 kb segment of DNA encoding a chimeric kanamycin resistance which permits selection of transformed plant cells. The chimeric gene (P-³⁵S/KAN/NOS 3′) consists of the cauliflower mosaic virus (CaMV) ³⁵S promoter, the neomycin phosphotransferase typeII (KAN) gene, and the 3′-nontranslated region of the nopaline synthase gene (NOS 3′) (Fraley et al., 1983). The next segment is the 0.75 kb oriV containing the origin of replication from the RK2 plasmid. It is joined to the 3.1 kb SailI to PvuI segment of pBR322 (ori322) which provides the origin of replication for maintenance in E. coli and the bom site for the conjugational transfer into the Agrobacterium tumefaciens cells. The next segment is the 0.36 kb PvuI to BciI from pTiT37 that carries the nopaline-type T-DNA right border (Fraley et al., 1985).

The pMON977 vector is the same as pMON981 except for the presence of the P-En-CaMV³5S promoter in place of the FMV³⁵S promoter (see below).

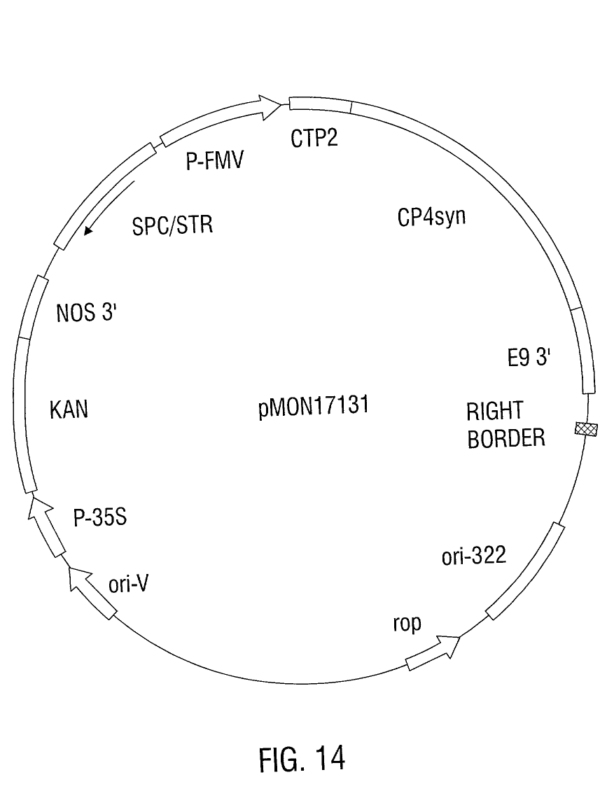

The pMON981 plasmid contains the following DNA segments: the 0.93 kb fragment isolated from transposon Tn7 encoding bacterial spectinomycin/streptomycin resistance (Spc/Str; a determinant for selection in E. coli and Agrobacteriurn tumefaciens (Fling et al., 1985)); the chimeric kanamycin resistance gene engineered for plant expression to allow selection of the transformed tissue. consisting of the 0.35 kb cauliflower mosaic virus 35S promoter (P-35S) (Odell et al., 1985), the 0.83 kb neomycin phosphotransferase typeli gene (KAN), and the 0.26 kb 3′-nontranslated region of the nopaline synthase gene (NOS 3′) (Fraley et al., 1983); the 0.75 kb origin of replication from the RK2 plasmid (oriV) (Stalker et al., 1981); the 3.1 kb SalI to PuuI segment of pBR322 which provides the origin of replication for maintenance in E. coli (ori-322) and the bom site for the conjugational transfer into the Agrobacterium tumefaciens cells, and the 0.36 kb PvuI to BclI fragment from the pTiT37 plasmid containing the nopaline-type T-DNA right border region (Fraley et al., 1985). The expression cassette consists of the 0.6 kb 35S promoter from the flgwort mosaic virus (P-FMV35S) (Gowda et al., 1989) and the 0.7 kb 3′ non-translated region of the pea rbcS-E9 gene (E9 3′) (Coruzzi et al., 1984, and Morelli et al., 1985). The 0.6 kb SspI fragment containing the FMV³⁵S promoter (FIG. 1) was engineered to place suitable cloning sites downstream of the transcriptional start site. The CTP2-CP4syn gene fusion was introduced into plant expression vectors (including pMON981, to form pMON17131; FIG. 14) and transformed into tobacco, canola, potato, tomato, sugarbeet, cotton, lettuce, cucumber, oil seed rape, poplar, and Arabidopsis.

The plant vector containing the Class II EPSPS gene may be mobilized into any suitable Agrobacterium strain for transformation of the desired plant species. The plant vector may be mobilized into an ABI Agrobacterium strain. A suitable ABI strain is the A208 Agrobacterium tumefaciens carrying the disarmed Ti plasmid pTiC58 (pMP9ORK) (Koncz and Schell, 1986). The Ti plasmid does not carry the T-DNA phytohormone genes and the strain is therefore unable to cause the crown gall disease. Mating of the plant vector into ABI was done by the triparental conjugation system using the helper plasmid pRK2013 (Ditta et al., 1980). When the plant tissue is incubated with the ABI::plant vector conjugate. the vector is transferred to the plant cells by the vir functions encoded by the disarmed pTiC58 plasmid. The vector opens at the T-DNA right border region. and the entire plant vector sequence may be inserted into the host plant chromosome. The pTiC58 Ti plasmid does not transfer to the plant cells but remains in the Agrobacterium.

Class II EPSPS free DNA vectors

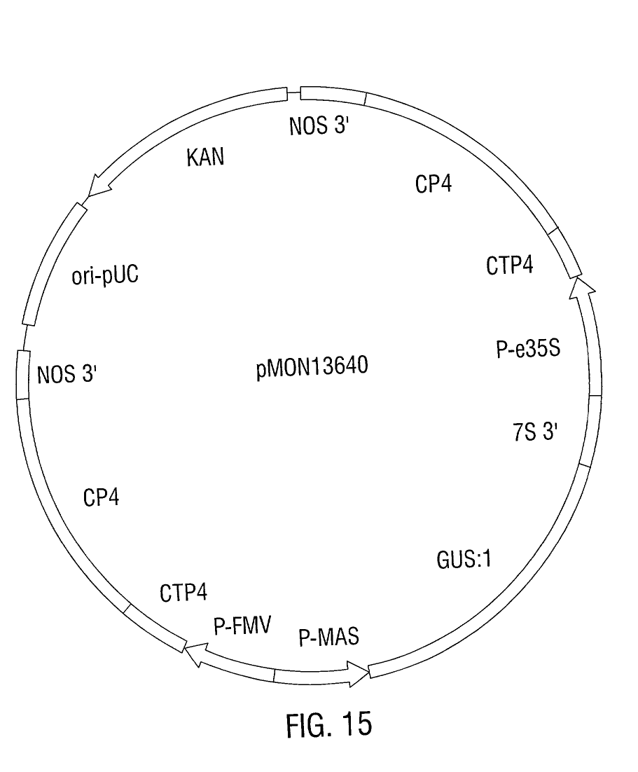

Class II EPSPS genes may also be introduced into plants through direct delivery methods. A number of direct delivery vectors were completed for the CP4 EPSPS gene. The vector pMON13640, a map of which is presented in FIG. 15, is described here. The plasmid vector is based on a pUC plasmid (Vieira and Messing, 1987) containing, in this case, the nptfl gene (kanamycin resistance; KAN) from Tn903 to provide a selectable marker in E. coli . The CTP4-EPSPS gene fusion is expressed from the P-FMV35S promoter and contains the NOS 3′ polyadenylation sequence fragment and from a second cassette consisting of the E³⁵S promoter, the CTP4-CP4 gene fusion and the NOS 3′ sequences. The scoreable GUS marker gene (Jefferson et al., 1987) is expressed from the mannopine synthase promoter (P-MAS; Velten et al., 1984) and the soybean 7S storage protein gene 3′ sequences (Schuler et al., 1982). Similar plasmids could also be made in which CTP-CP4 EPSPS fusions are expressed from the enhanced CaMV³⁵S promoter or other plant promoters. Other vectors could be made that are suitable for free DNA delivery into plants and such are within the skill of the art and contemplated to be within the scope of this disclosure.

Plastid transformation

While transformation of the nuclear genome of plants is much more developed at this time. a rapidly advancing alternative is the transformation of plant organelles. The transformation of plastids of land plants and the regeneration of stable transformants has been demonstrated (Svab et al., 1990; Maliga et al., 1993). Transformants are selected, following double cross-over events into the plastid genome, on the basis of resistance to spectinomycin conferred through rRNA changes or through the introduction of an aminoglycoside 3′-adenyltransferase gene (Svab et al. 1990: Svab and Maliga, 1993), or resistance to kanamycin through the neomycin phosphotransferase NptII (Carrer et al., 1993). DNA is introduced by biolistic means (Svab et al, 1990; Maliga et al., 1993) or by using polyethylene glycol (O'Neill et al., 1993). This transformation route results in the production of 500-10,000 copies of the introduced sequence per cell and high levels of expression of the introduced gene have been reported (Carrer et al., 1993; Maliga et al., 1993). The use of plastid transformation offers the adavantages of not requiring the chloroplast transit peptide signal sequence to result in the localization of the heterologous Class II EPSPS in the chloroplast and the potential to have many copies of the heterologous plant-expressible Class II EPSPS gene in each plant cell since at least one copy of the gene would be in each plastid of the cell.

Plant Regeneration

When expression of the Class II EPSPS gene is achieved in transformed cells (or protoplasts), the cells (or protoplasts) are regenerated into whole .plants. Choice of methodology for the regeneration step is not critical, with suitable protocols being available for hosts from Leguminosae (alfalfa, soybean, clover, etc.), Umbelliferae (carrot, celery, parsnip), Cruciferae (cabbage, radish, rapeseed, etc.), Cucurbitaceae (melons and cucumber), Gramineae (wheat, rice. corn, etc.), Solanaceae (potato, tobacco, tomato, peppers), various floral crops as well as various trees such as poplar or apple, nut crops or vine plants such as grapes. See, e.g., Ammirato, 1984; Shimamoto, 1989; Fromm, 1990; Vasil, 1990.

The following examples are provided to better elucidate the practice of the present invention and should not be interpreted in any way to limit the scope of the present invention. Those skilled in the art will recognize that various modifications, truncations, etc. can be made to the methods and genes described herein while not departing from the spirit and scope of the present invention.

In the examples that follow, EPSPS activity in plants is assayed by the following method. Tissue samples were collected and immediately frozen in liquid nitrogen. One gram of young leaf tissue was frozen in a mortar with liquid nitrogen and ground to a fine powder with a pestle. The powder was then transferred to a second mortar, extraction buffer was added (1 ml /gram), and the sample was ground for an additional 45 seconds. The extraction buffer for canola consists of 100 mM Tris, 1 mM EDTA, 10 % glycerol, 5 mM DTr, 1 mM BAM, 5 mM ascorbate, 1.0 mg/ml BSA, pH 7.5 (4° C.). The extraction buffer for tobacco consists of 100 mM Tris, 10 mM EDTA, 35 mM KCI, 20 % glycerol, 5 mM DTT, 1 mM BAM, 5 mM ascorbate, 1.0 mg/ml BSA, pH 7.5 (4° C.). The mixture was transferred to a microfuge tube and centrifiged for 5 minutes. The resulting supernatants were desalted on spin G-50 (Pharmacia) columns, previously equilibrated with extraction buffer (without BSA), in 0.25 ml aliquots. The desalted extracts were assayed for EPSP synthase activity by radioactive HPLC assay. Protein concentrations in samples were determined by the BioRad microprotein assay with BSA as the standard.

Protein concentrations were determined using the BioRad Microprotein method. BSA was used to generate a standard curve ranging from 2 -24 μg. Either 800 μl of standard or diluted sample was mixed with 200 μl of concentrated BioRad Bradford reagent. The samples were vortexed and read at A(595) after 5 minutes and compared to the standard curve.

EPSPS enzyme assays contained HEPES (50 mM), shikimate-3-phosphate (2 mM), NH4 molybdate (0.1 mM) and KF (5 mM), with or without glyphosate (0.5 or 1.0 mM). The assay mix (30 RI) and plant extract (10 μl) were preincubated for 1 minute at 25° C. and the reactions were initiated by adding 14C-PEP (1 mM). The reactions were quenched after 3 minutes with 50 μl of 90% EtOH/0.lM HOAc, pH 4.5. The samples were spun at 6000 rpm and the resulting supernatants were analyzed for 14C-EPSP production by HPLC. Percent resistant EPSPS is calculated from the EPSPS activities with and without glyphosate.

The percent conversion of ¹⁴C labeled PEP to ¹⁴C EPSP was determined by HPLC radioassay using a C18 guard column (Brownlee) and an AX₁₀₀ HPLC column (0.4×25 cm, Synchropak) with 0.28 M isocratic potassium phosphate eluant, pH 6.5, at 1 ml/min. Initial velocities were calculated by multiplying fractional turnover per unit time by the initial concentration of the labeled substrate (1 mM). The assay was linear with time up to ˜3 minutes and 30% turnover to EPSPS. Samples were diluted with 10 mM Tris, 10% glycerol, 10 mM DTT, pH 7.5 (4° C.) if necessary to obtain results within the linear range.

In these assays DLdithiotheitol (DTT), benzamidine (BAM), and bovine serum albumin (BSA, essentially globulin free) were obtained from Sigma. Phosphoenolpyruvate (PEP) was from Boehringer Mannheim and phosphoenol-[1-¹⁴C]pyruvate (28 mCi/mmol) was from Amersham.

### EXAMPLES

### Example 1

Transformed tobacco plants have been generated with a number of the Class II EPSPS gene vectors containing the CP4 EPSPS DNA sequence as described above with suitable expression of the EPSPS. These transformed plants exhibit glyphosate tolerance imparted by the Class II CP4 EPSPS.

Transformation of tobacco employs the tobacco leaf disc transformation protocol which utilizes healthy leaf tissue about 1 month old. After a 15-20 minutes surface sterilization with 10% Clorox plus a surfactant, the leaves are rinsed 3 times in sterile water. Using a sterile paper punch, leaf discs are punched and placed upside down on MS104 media (MS salts 4.3 g/l, sucrose 30 g/l, B5 vitamins 500X 2 mill, NAA 0.1 mgtl, and BA 1.0 mg/l) for a 1 day preculture.

The discs are then inoculated with an overnight culture of a disarmed Agrobacternum ABI strain containing the subject vector that had been diluted 1/5 (i.e.: about 0.6 OD). The inoculation is done by placing the discs in centrifuge tubes with the culture. After 30 to 60 seconds, the liquid is drained off and the discs were blotted between sterile filter paper. The discs are then placed upside down on MS104 feeder plates with a filter disc to cture.

After 2-3 days of co-culture, the discs are transferred, still upside down, to selection plates with MS104 media. After 2-3 weeks, callus tissue formed, and individual clumps are separated from the leaf discs. Shoots are cleanly cut from the callus when they are large enough to be distinguished from stems. The shoots are placed on hormone-free rooting media (MSO: MS salts 4.3 g/l, sucrose 30 g/l, and B5 vitamins 500X 2 ml/l) with selection for the appropriate antibiotic resistance. Root formation occurred in 1-2 weeks. Any leaf callus assays are preferably done on rooted shoots while still sterile. Rooted shoots are then placed in soil and kept in a high humidity environment (i.e.: plastic containers or bags). The shoots are hardened off by gradually exposing them to ambient humidity conditions.

Expression of CP4 EPSPS protein in transformed plants

Tobacco cells were transformed with a number of plant vectors containing the native CP4 EPSPS gene, and using different promoters andlor CTP's. Preliminary evidence for expression of the gene was given by the ability of the leaf tissue from antibiotic selected transformed shoots to recallus on glyphosate. In some cases, glyphosate-tolerant callus was selected directly following transformation. The level of expression of the CP4 EPSPS was determined by the level of glyphosate-tolerant EPSPS activity (assayed in the presence of 0.5 mM glyphosate) or by Western blot analysis using a goat anti-CP4 EPSPS antibody. The Western blots were quantitated by densitometer tracing and comparison to a standard curve established using purified CP4 EPSPS. These data are presented as % soluble leaf protein. The data from a number of transformed plant lines and transformation vectors are presented in Table VI below.

TABLE VI
Expression of CP4 EPSPS in transformed tobacco tissue

| | | CP4 EPSPS ** |
|---|---|---|
| Vector | Plant # | (% leaf protein) |
| pMON17110 | 25313 | 0.02 |
| pMON17110 | 25329 | 0.04 |
| pMON17116 | 25095 | 0.02 |
| pMON17119 | 25106 | 0.09 |
| pMON17119 | 25762 | 0.09 |
| pMON17119 | 25767 | 0.03 |

** Glyphosate-tolerant EPSPS activity was also demonstrated in leaf extracts for these plants.

Glyphosate tolerance has also been demonstrated at the whole plant level in transformed tobacco plants. In tobacco, R₀ transformants of CTP2-CP4 EPSPS were sprayed at 0.4 lb/acre (0.448 kg/hectare), a rate sufficient to kill control non-transformed tobacco plants corresponding to a rating of 3. 1 and 0 at days 7, 14 and 28. respectively, and were analyzed vegetatively and reproductively (Table VII).

TABLE VII
Glyphosate tolerance in Rₒ tobacco CP4 transformants

| | Score** | | | |
|---|---|---|---|---|
| | Vegetative | | | |
| Vector/Plant # | day 7 | day 14 | day 28 | Fertile |
| pMON17110/25313 | 6 | 4 | 2 | no |
| pMON17110/25329 | 9 | 10 | 10 | yes |
| pMON17119/25106 | 9 | 9 | 10 | yes |

*Spray rate = 0.4 lb/acre (0.448 kg/hectare)
**Plants are evaluated on a numerical scoring system of 0-10 where a vegetative score of 10 represents no damage relative to nonsprayed controls and 0 represents a dead plant. Reproductive scores (Fertile) are determined at 28 days after spraying and are evaluated as to whether or not the plant is fertile.

### Example 2A

Canola plants were transformed with the pMON17110, pMON17116, and pMON17131 vectors and a number of plant lines of the transformed canola were obtained which exhibit glyphosate tolerance.

Plant Material

Seedlings of Brassica napus cv Westar were established in 2 inch (˜5 cm) pots containing Metro Mix 350. They were grown in a growth chamber at 24° C., 16/8 hour photoperiod, light intensity of 400 uEm-²sec-i (HID lamps) They were fertilized with Peters 20-10-20 General Purpose Special. After 2½ weeks they were transplanted to 6 inch (˜15 cm) pots and grown in a growth chamber at 15/10° C. day/night temperature. 16/8 hour photoperiod, light intensity of 800 uEm-²see-I (HID lamps). They were fertilized with Peters 15-30-15 Hi-Phos Special.

Transformation/Selection/Regeneration

Four terminal internodes from plants just prior to bolting or in the process of bolting but before flowering were removed and surfaced sterilized in 70% v/v ethanol for 1 minute, 2% w/v sodium hypochlorite for 20 minutes and rinsed 3 times with sterile deionized water. Stems with leaves attached could be refrigerated in moist plastic bags for up to 72 hours prior to sterilization. Six to seven stem segments were cut into 5mm discs with a Redco Vegetable Slicer 200 maintaining orientation of basal end.

The Agrobacterium was grown overnight on a rotator at 24° C. in 2 mls of Luria Broth containing 50 mg/l kanamycin, 24mg/l chloramphenicol and 100 mg/l spectinomycin. A 1:10 dilution was made in MS (Murashige and Skoog) media giving approximately 9xlO⁸ cells per ml. This was confirmed with optical density readings at 660 mu. The stem discs (explants) were inoculated with 1.0 ml of Agrobacterium and the excess was aspirated from the explants.

The explants were placed basal side down in petri plates containing 1/10X standard MS salts. B5 vitamins. 3% sucrose. 0.8% agar, pH 5.7, 1.0 mg/l 6-benzyladenine (BA). The plates were layered with 1.5 ml of media containing MS salts, B5 vitamins, 3% sucrose, pH 5.7, 4.0 mg/i p-chlorophenoxyacetic acid, 0.005 mg/l kinetin and covered with sterile filter paper.

Following a 2 to 3 day co-culture, the explants were transferred to deep dish petri plates containing MS salts, B5 vitamins, 3% sucrose, 0.8% agar, pH 5.7, 1 mg/l BA. 500 mg/l carbenicillin, 50mg/l cefotaxime, 200 mWi kanamycin or 175 mg/l gentamicin for selection. Seven explants were placed on each plate. After 3 weeks they were transferred to fresh media. 5 explants per plate. The explants were cultured in a growth room at 25° C. continuous light (Cool White).

Expression Assay

After 3 weeks shoots were excised from the explants. Leaf recallusing assays were initiated to confirm modification of Re shoots. Three tiny pieces of leaf tissue were placed on recallusing media containing MS salts, B5 vitamins, 3% sucrose, 0.8% agar, pH 5.7, 5.0mg/l BA, 0.5 mg/l naphthalene acetic acid (NAA), 500 mg/l carbenicillin, 50mg/l cefotaxime and 200 mg/l kanamycin or gentamicin or 0.5 mM glyphosate. The leaf assays were incubated in a growth room under the same conditions as explant culture. After 3 weeks the leaf recallusing assays were scored for herbicide tolerance (callus or green leaf tissue) or sensitivity (bleaching).

Trnpantation

At the time of excision, the shoot stems were dipped in Rootone® and placed in 2 inch (˜5 cm) pots containing Metro-Mix 350 and placed in a closed humid environment. They were placed in a growth chamber at 24° C., 16/8 hour photoperiod, 400 uEm-¹sec-²(HID lamps) for a hardening-off period of approximately 3 weeks.

The seed harvested from R₀, plants is R₁ seed which gives rise to R₁ plants. To evaluate the glyphosate tolerance of an Ro plant, its progeny are evaluated. Because an R₀ plant is assumed to be hemizygous at each insert location, selfing results in maximum genotypic segregation in the R₁. Because each insert acts as a dominant allele, in the absence of linkage and assuming only one hemizygous insert is required for tolerance expression. one insert would segregate 3:1, two inserts, 15:1, three inserts 63:1, etc. Therefore, relatively few R₁ plants need be grown to find at least one resistant phenotype.

Seed from an R₀ plant is harvested, threshed, and dried before planting in a glyphosate spray test. Various techniques have been used to grow the plants for R₁ spray evaluations. Tests are conducted in both greenhouses and growth chambers. Two planting systems are used; ˜10 cm pots or plant trays containing 32 or 36 cells. Soil used for planting is either Metro 350 plus three types of slow release fertilizer or plant Metro 350. Irrigation is either overhead in greenhouses or sub-irrigation in growth chambers. Fertilizer is applied as required in irrigation water. Temperature regimes appropriate for canola were maintained. A sixteen hour photoperiod was maintained. At the onset of flowering, plants ore transplanted to ˜15 cm pots for seed production.

A spray “batch” consists of several sets of R₁ progenies all sprayed on the same date. Some batches may also include evaluations of other than R₁ plants. Each batch also includes sprayed and unsprayed non-transgenic genotypes representing the genotypes in the particular batch which were putatively transformed. Also included in a batch is one or more non-segregating transformed genotypes previously identified as having some resistance.

Two-six plants from each individual Ro progeny are not sprayed and serve as controls to compare and measure the glyphosate tolerance, as well as to assess any variability not induced by the glyphosate. When the other plants reach the 2-4 leaf stage, usually 10 to 20 days after planting, glyphosate is applied at rates varying from 0.28 to 1.12 kg/ha, depending on objectives of the study. Low rate technology using low volumes has been adopted. A laboratory track sprayer has been calibrated to deliver a rate equivalent to field conditions.

A scale of 0 to 10 is used to rate the sprayed plants for vegetative resistance. The scale is relative to the unsprayed plants from the same R₀ plant. A 0 is death, while a 10 represents no visible difference from the unsprayed plant. A higher number between 0 and 10 represents progressively less damage as compared to the unsprayed plant. Plants are scored at 7, 14, and 28 days after treatment (DAT), or until bolting, and a line is given the average score of the sprayed plants within an R₀ plant family.

Six integers are used to qualitatively describe the degree of reproductive damage from glyphosate:

0: No floral bud development

2: Floral buds present, but aborted prior to opening

4: Flooers open, but no anthers, or anthers fail to extrude past petals

6: Sterile anthers

8: Partially sterile anthers

10: Fully fertile flowers

Plants are scored using this scale at or shortly after initiation of flowering, depending on the rate of floral structure development.

Expression of EPSPS in Canola

After the 3 week period, the transformed canola plants were assayed for the presence of glyphosate-tolerant EPSPS activity (assayed in the presence of glyphosate at 0.5 mM). The results are shown in Table VIII.

TABLE VIII
Expression of CP4 EPSPS in transformed Canola plants

| | | % resistant EPSPS activity |
|---|---|---|
| | | of Leaf extract |
| | Plant # | (at 0.5 mM glyphosate) |
| Vector Control | | 0 |
| pMON17110 | 41 | 47 |
| pMON17110 | 52 | 28 |
| pMON17110 | 71 | 82 |
| pMON17110 | 104 | 75 |
| pMON17110 | 172 | 84 |
| pMON17110 | 177 | 85 |
| pMON17110 | 252 | 29* |
| pMON17110 | 350 | 49 |
| pMON17116 | 40 | 25 |
| pMON17116 | 99 | 87 |
| pMON17116 | 175 | 94 |
| pMON17116 | 178 | 43 |
| pMON17116 | 182 | 18 |
| pMON17116 | 252 | 69 |
| pMON17116 | 298 | 44* |
| pMON17116 | 332 | 89 |
| pMON17116 | 383 | 97 |
| pMON17116 | 395 | 52 |

*assayed in the presence of 1.0 mM glyphosate

R₁ transformants of canola were then grown in a growth chamber and sprayed with glyphosate at 0.56 kg/ha tkilogram/hectare) and rated vegetatively. These results are shown in Table IXA-IXC. It is to be noted that expression of glyphosate resistant EPSPS in all tissues is preferred to observe optimal glyphosate tolerance phenotype in these transgenic plants. In the Tables below, only expression results obtained with leaf tissue are described.

TABLE IXA
Glyphosate tolerance in Class II EPSPS
canola R₁ transformants
(pMON17110 = P-E35S; pMON17116 = P-FMV35S; R1 plants;
Spray rate 0.56 kg/ha)

| | |
|---|---|
| | Vegetative |
| % resistant | Score** |

| Vector/Plant No. | EPSPS* | day 7 | day 14 |
|---|---|---|---|
| Control Westar | 0 | 5 | 3 |
| pMON17110/41 | 47 | 6 | 7 |
| pMON17110/71 | 82 | 6 | 7 |
| pMON17110/177 | 85 | 9 | 10 |
| pMON17116/40 | 25 | 9 | 9 |
| pMON17116/99 | 87 | 9 | 10 |
| pMON17116/175 | 94 | 9 | 10 |
| pMON17116/178 | 43 | 6 | 3 |
| pMON17116/182 | 18 | 9 | 10 |
| pMON17116/383 | 97 | 9 | 10 |

TABLE IXB
Glyphosate tolerance in Class II EPSPS
canola R₁ transformants
(pMON17131 = P-FMV35S; R1 plants; Spray rate = 0.84 kg/ha)

| | Vegetative score** | Reproductive score |
|---|---|---|
| Vector/Plant No. | day 14 | day 28 |
| 17131/78 | 10 | 10 |
| 17131/102 | 9 | 10 |
| 17131/115 | 9 | 10 |
| 17131/116 | 9 | 10 |
| 17131/157 | 9 | 10 |
| 17131/169 | 10 | 10 |
| 17131/255 | 10 | 10 |
| control Westar | 1 | 0 |

TABLE IXC
Glyphosate tolerance in Class I EPSPS
canola transformants
(P-E35S: R2 Plants; Spray rate = 0.28 kg/ha)

| | |
|---|---|
| | Vegetative |
| % resistant | Score** |

| Vector/Plant No. | EPSPS* | day 7 | day 14 |
|---|---|---|---|
| Control Westar | 0 | 4 | 2 |
| pMON899/715 | 96 | 5 | 6 |
| pMON899/744 | 95 | 8 | 8 |
| pMON899/794 | 86 | 6 | 4 |
| pMON899/818 | 81 | 7 | 8 |
| pMON899/885 | 57 | 7 | 6 |

*% resistant EPSPS activity in the presence of 0.5 mM glyphosate
**A vegetative score of 10 indicates no damage, a score of 0 is given to a dead plant.

The data obtained for the Class II EPSPS transformants may be compared to glyphosate-tolerant Class I EPSP transformants in which the same promoter is used to express the EPSPS genes and in which the level of glyphosate-tolerant EPSPS activity was comparable for the two types of transformants. A comparison of the data of pMON17110 [in Table IXA] and pMON17131 [Table IXB] with that for pMON899 [in Table IX]; the Class I gene in pMON899 is that from A. thaliana [Klee et al., 1987] in which the glycine at position 101 was changed to an alaninej illustrates that the Class II EPSPS is at least as good as that of the Class I EPSPS. An improvement in vegetative tolerance of Class II EPSPS is apparent when one takes into account that the Class II plants were sprayed at twice the rate and were tested as R₁ plants.

### Example 2B

The construction of two plant transformation vectors and the transformation procedures used to produce glyphosate-tolerant canola plants are described in this example The vectors, pMON17209 and pMON17237, were used to generate transgenic glyphosate-tolerant canola lines. The vectors each contain the gene encoding the 5-enol-pyruvyl-shikimate-3-phosphate synthase (EPSPS) from Agrobacterium sp. strain CP4. The vectors also contain either the gox gene encoding the glyphosate oxidoreductase enzyme (GOX) from Achromobacter sp. strain LBAA (Barry et al., 1992) or the gene encoding a variant of GOX (GOX v.247) which displays improved catalytic properties. These enzymes convert glyphosate to aminomethylphosphonic acid and glyoxvlate and protect the plant from damage by the metabolic inactivation of glyphosate. The combined result of providing an alternative, resistant EPSPS enzyme and the metabolism of glyphosate produces transgenic plants with enhanced tolerance to glyphosate

Molecular biology techniques. In general. standard molecular biology and microbial genetics approaches were employed (Maniatis et al., 1982). Site-directed mutageneses were carried out as described by Kunkel et al. (1987). Plant-preferred genes were synthesized and the sequence confirmed.

Plant transformation vectors. The following describes the general features of the plant transformation vectors that were modified to form vectors pMON17209 and pMON17237. The Agrobacterium mediated plant transformation vectors contain the following well-characterized DNA segments which are required for replication and function of the plasmids (Rogers and Klee, 1987; KIee and Rogers, 1989). The first segment is the 0.45 kb Clal-Dral fragment from the pTi15955 octopine Ti plasmid which contains the T-DNA left border region (Barker et al., 1983). It is joined to the 0.75 kb origin of replication (oriV) derived from the broad-host range plasmid RK2 (Stalker et al., 1981). The next segment is the 3.1 kb Sall-PvuI segment of pBR322 which provides the origin of replication for maintenance in E. coli and the bom site for the conjugational transfer into the Agrobacterium tumefaciens cells (Bolivar et al., 1977). This is fused to the 0.93 kb fragment isolated from transposon Tn7 which encodes bacterial spectinomycin and streptomycin resistance (Fling et al., 1985), a determinant for the selection of the plasmids in E. coli and Agrobacterium. It is fused to the 0.36 kb PvuI-BclI fragment from the pTiT37 plasmid which contains the nopaline-tvpe T-DNA right border region (Fraley et al., 1985). Several chimeric genes engineered for plant expression can be introduced between the Ti right and left border regions of the vector. In addition to the elements described above, this vector also includes the 35S promoter/NPTII/NOS 3′ cassette to enable selection of transformed plant tissues on kanamycin (Klee and Rogers, 1989; Fraley et al., 1983; and Odell, et al. 1985) within the borders. An “empty” expression cassette is also present between the borders and consists of the enhanced E35S promoter (Kay et al. 1987), the 3′ region from the small subunit of RUBPcarboxylase of pea (E9) (Coruzzi et al . 1984; Morelli et al., 1986), and a number of restriction enzyme sites that may be used for the cloning of DNA sequences for expression in plants. The plant transformation system based on Agrobacterium tumefaciens delivery has been reviewed (Klee and Rogers, 1989; Fraley et al., 1986). The Agrobacterium mediated transfer and integration of the vector T-DNA into the plant chromosome results in the expression of the chimeric genes conferring the desired phenotype in plants.

Bacterial Inoculum. The binary vectors are mobilized into Agrobacterium tumefaciens strain ABI by the triparental conjugation system using the helper plasmid pRK2013 (Ditta et al. 1980). The ABI strain contains the disarmed pTiC58 plasmid pMP9ORK (Koncz and Schell, 1986) in the chloramphenicol resistant derivative of the Agrobacterium tumefaciens strain A208.

Transformation procedure. Agrobacterium inocula were grown overnight at 28° C. in 2 ml of LBSCK (LBSCK is made as follows: LB liquid medium [1 liter volume] =10 g NaCl; 5 g Yeast Extract;10 g tryptone; pH 7.0, and autoclave for 22 minutes. After autoclaving, add spectinomycin (50 mg/ml stock)—2 ml, kanamycin (50 mg/ml stock)—1 ml, and chloramphenicol (25 mg/ml stock)—1 ml.). One day prior to inoculation, the Agrobacterium was subcultured by inoculating 200ul into 2 ml of fresh LBSCK and grown overnight. For inoculation of plant material, the culture was diluted with MSO liquid medium to an A₆₆₀ range of 0.2-0.4.

Seedlings of Brassica napus cv. Westar were grown in Metro Mix 350 (Hummert Seed Co., St. Louis, Mo.) in a growth chamber with a day/night temperature of 15/10° C., relative humidity of 50%, 16h/8h photoperiod, and at a light intensity of 500 ;mol m ² sec-¹. The plants were watered daily (via sub-irngation) and fertilized every other day with Peter's 15:30:15 (Fogelsville, Pa.).

In general, all media recipes and the transformation protocol follow those in Fry et. aL (1987). Five to six week-old Westar plants were harvested when the plants had bolted (but prior to flowering), the leaves and buds were removed, and the 4-5 inches of stem below the flower buds were used as the explant tissue source. Following sterilization with 70% ethanol for 1 min and 38% Clorox for 20 min, the stems were rinsed three times with sterile water and cut into 5 mm-long segments (the orientation of the basal end of the stem segments was noted). The plant material was incubated for 5 minutes with the diluted Agrobacterium culture at a rate of 5 ml of culture per 5 stems. The suspension of bacteria was removed by aspiration and the explants were placed basal side down -for an optimal shoot regeneration response—onto co-culture plates (1/10 MSO solid medium with a 1.5 ml IXD (tobacco xanthi diploid) liquid medium overlay and covered with a sterile 8.5 cm filter paper). Fifty-to-sixty stem explants were placed onto each co-culture plate.

After a 2 day co-culture period, stem explants were moved onto MS medium containing 750 mg/l carbenicillin, 50 mg/l cefotaxime, and 1 mg/l BAP (benzylaminopurine) for 3 days. The stem explants were then placed for two periods of three weeks each, again basal side down and with 5 explants per plate, onto an MS/0.1 mM glyphosate, selection medium (also containing carbenicillin, cefotaxime, and BAP (The glyphosate stock [0.5M] is prepared as described in the following: 8.45 g glyphosate [analytical grade] is dissolyed in 50 ml deionized water, adding KOH pellets to dissolye the glyphosate, and the volume is brought to 100 ml following adjusting the pH to 5.7. The solution is filter-sterilized and stored at 4° C). After 6 weeks on this glyphosate selection medium, green. normally developing shoots were excised from the stem explants and were placed onto fresh MS medium containing 750 mg/l carbenicillin. 50 mgil cefotaxime, and 1 mg/l BAP, for further shoot development. When the shoots were 2-3 inches tall, a fresh cut at the end of the stem was made, the cut end was dipped in Root-tone, and the shoot was placed in Metro Mix 350 soil and allowed to harden-off for 2-3 weeks.

Construction of Canola transformation vector pMON17209.

The EPSPS gene was isolated originally from Agrobacterium sp. strain CP4 and expresses a highly tolerant enzyme. The original gene contains sequences that could be inimical to high expression of the gene in some plants. These sequences include potential polyadenylation sites that are often A+T rich, a higher G+C% than that frequently found in dicotyledonous plant genes (63% versus ˜50%), concentrated stretches of G and C residues, and codons that may not used frequently in dicotyledonous plant genes. The high G+C% in the CP4 EPSPS gene could also result in the formation of strong hairpin structures that may affect expression or stability of the RNA. A plant preferred version of the gene was synthesized and used for these vectors. This coding sequence was expressed in E. coli from a PRerA-genelOL vector (Olins et al., 1988) and the EPSPS activity was compared with that from the native CP4 EPSPS gene. The appkₘfor PEP for the native and synthetic genes was 11.8 μM and 12.7 μM, respectively, indicating that the enzyme expressed from the synthetic gene was unaltered. The N-terminus of the coding sequence was then mutagenized to place an SphI site (GCATGC) at the ATG to permit the construction of the CTP2-CP4 synthetic fusion for chloroplast import. This change had no apparent effect on the in vivo activity of CP4 EPSPS in E. coli as judged by complementation of the aroA mutant. A CTP-CP4 EPSPS fusion was constructed between the Arabidopsis thaliana EPSPS CTP (Klee et al., 1987) and the CP4 EPSPS coding sequences. The Arabidopsis CTP was engineered by site-directed mutagenesis to place a SphI restriction site at the CTP processing site. This mutagenesis replaced the Glu-Lys at this location with Cys-Met. The CTP2-CP4 EPSPS fusion was tested for import into chloroplasts isolated from Lactuca sativa using the methods described previously (della-Cioppa et al., 1986: 1987).

The GOX gene that encodes the glyphosate metabolizing enzyme glyphosate oxidoreductase (GOX) was cloned originally from Achromobacter sp. strain LBAA (Hallas et al., 1988; Barry et al., 1992). The gox gene from strain LBAA was also resynthesized in a plant-preferred sequence version and in which many of the restriction sites were removed (PCT Appin. No. WO 92100377). The GOX protein is targeted to the plastids by a fusion between the C-terminus of a CTP and the N-terminus of GOM A CTP, derived from the SSUIA gene from Arabidopsis thaliana (Timko et al., 1988) was used. This CTP (CTP1) was constructed by a combination of site-directed mutageneses. The CTP1 is made up of the SSULA CTP (amino acids 1-55), the first 23 amino acids of the mature SSUIA protein (56-78), a serine residue (amino acid 79), a new segment that repeats amino acids 50 to 56 from the CTP and the first two from the mature protein (amino acids 80-87), and an alanine and methionine residue (amino acid 88 and 89). An NcoI restriction site is located at the 3′ end (spans the Met89 codon) to facilitate the construction of precise fusions to the 5′ of GOX. At a later stage, a BglII site was introduced upstream of the N-terminus of the SSU1A sequences to facilitate the introduction of the fusions into plant transformation vectors. A fusion was assembled between CTP1 and the synthetic GOX gene.

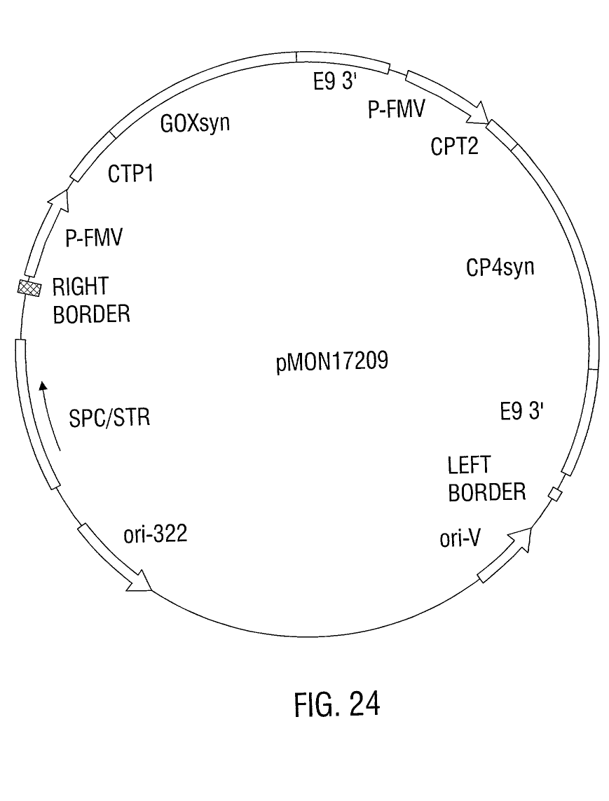

The CP4 EPSPS and GOX genes were combined to form pMON17209 as described in the following. The CTP2-CP4 EPSPS fusion was assembled and inserted between the constitutive FMV³⁵S promoter (Gowda et al., 1989; Richins et al., 1987) and the E9 3′ region (Coruzzi et al., 1984; Morelli et al., 1985) in a pUC vector (Yannisch-Perron et al. 1985; Vieira and Messing, 1987) to form pMON17190; this completed element may then be moved easily as a NotI—NotI fragment to other vectors. The CTP1-GOX fusion was also assembled in a pUC vector with the FMV³⁵S promoter. This element was then moved as a HindlII-BamHI fragment into the plant transformation vector pMON10098 and joined to the E9 3′ region in the process. The resultant vector pMON17193 has a single NotI site into which the FMV ³⁵SICTP2-CP4 EPSPSIE9 3′ element from pMON17190 was cloned to form pMON17194. The kanamycin plant transformation selection cassette (Fraley et al., 1985) was then deleted from pMON17194, by cutting with XhoI and religating, to form the pMON17209 vector (FIG. 24).

Construction of Canola transformation vector pMON17237.

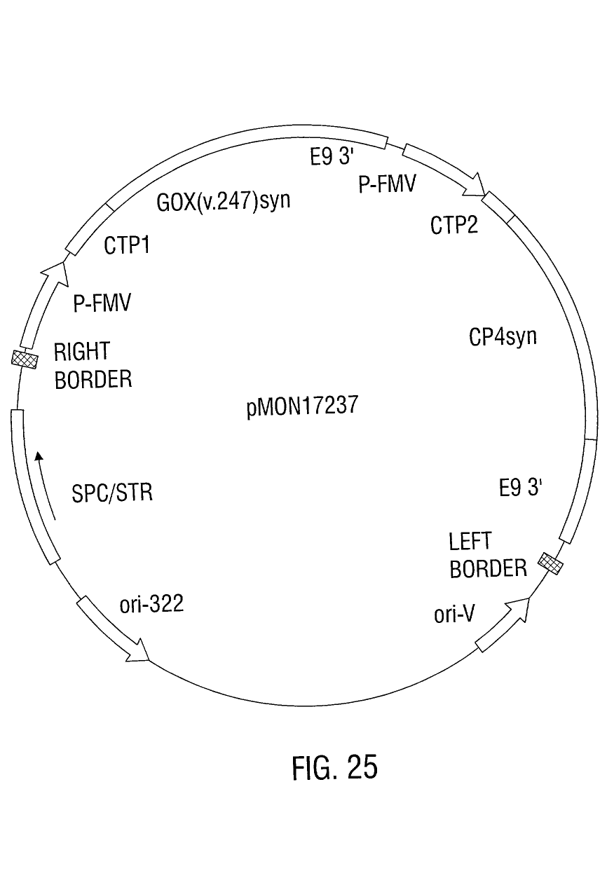

TheGOX enzyme has an apparent Kₘ for glyphosate [appkₘ(glyphosate)] of −25 mM. In an effort to improve the effectiveness of the glyphosate metabolic rate in planta, a variant of GOX has been identified in which the appkₘ(glyphosate) has been reduced approximately 10-fold; this variant is referred to as GOX v.247 and the sequence differences between it and the original plant-preferred GOX are illustrated in PCT Appln. No. WO 92100377. The GOX v.247 coding sequence was combined with CTP1 and assembled with the FMV35S promoter and the E9 3′ by cloning into the pMON17227 plant transformation vector to form pMON17241. In this vector, effectively, the CP4 EPSPS was replaced by GOX v.247. The pMON17227 vector had been constructed by replacing the CTP1-GOX sequences in pMON17193 with those for the CTP2-CP4 EPSPS, to form pMON17199 and followed by deleting the kanamycin cassette (as described above for pMON17209). The pMON17237 vector (FIG. 25) was then completed by cloning the FMV³⁵S/CTP2-CP4 EPSPS/E9 3′ element as a NotI—NotI fragment into pMON17241.

### Example 8

Soybean plants were transformed with the pMON13640 (FIG. 15) vector and a number of plant lines of the transformed soybean were obtained which exhibit glyphosate tolerance.

Soybean plants are transformed with pMON13640 by the method of microprojectile injection using particle gun technology as described in Christou et al. (1988). The seed harvested from R₀ plants is R₁ seed which gives rise to R₁ plants. To evaluate the glyphosate tolerance of an R₀ plant, its progeny are evaluated. Because an R₀ plant is assumed to be hemizygous at each insert location, selfing results in maximum genotypic segregation in the RI. Because each insert acts as a dominant allele, in the absence of linkage and assuming only one hemizygous insert is required for tolerance expression, one insert would segregate 3:1, two inserts, 15:1, three inserts 63:1, etc. Therefore, relatively few R₁ plants need be grown to find at least one resistant phenotype.

Seed from an R₀ soybean plant is harvested, and dried before planting in a glyphosate spray test. Seeds are planted into 4 inch (˜5 cm) square pots containing Metro 350. Twenty seedlings from each Ro plant is considered adequate for testing. Plants are maintained and grown in a greenhouse environment. A 12.5-14 hour photoperiod and temperatures of 30° C. day and 24° C. night is regulated. Water soluble Peters Pete Lite fertilizer is applied as needed.

A spray “batch” consists of several sets of R₁ progenies all sprayed on the same date. Some batches may also include evaluations of other than R₁ plants. Each batch also includes sprayed and unsprayed non-transgenic genotypes representing the genotypes in the particular batch which were putatively transformed. Also included in a batch is one or more non-segregating transformed genotypes previously identified as having some resistance.

One to two plants from each individual R₀ progeny are not sprayed and serve as controls to compare and measure the glyphosate tolerance, as well as to assess any variability not induced by the glyphosate. When the other plants reach the first trifoliate leaf stage, usually 2-3 weeks after planting, glyphosate is applied at a rate equivalent of 128 oz./acre (8.895 kg/ha) of Roundup®. A laboratory track sprayer has been calibrated to deliver a rate equivalent to those conditions.

A vegetative score of 0 to 10 is used. The score is relative to the unsprayed progenies from the same R₀ plant. A 0 is death, while a 10 represents no visible difference from the unsprayed plant. A higher number between 0 and 10 represents progressively less damage as compared to the unsprayed plant. Plants are scored at 7, 14, and 28 days after treatment (DAT). The data from the analysis of one set of transformed and control soybean plants are described on Table X and show that the CP4 EPSPS gene imparts glyphosate tolerance in soybean also.

TABLE X
Glyphosate tolerance in Class II EPSPS soybean
transformants
(P-E35S, P-FMV35S; RO plants; Spray rate = 128 oz./acre)

| | Vegetative score | | |
|---|---|---|---|
| Vector/Plant No. | day 7 | day 14 | day 28 |
| 13640/40-11 | 5 | 6 | 7 |
| 13640140-3 | 9 | 10 | 10 |
| 13640/40-7 | 4 | 7 | 7 |
| control A54032 | 1 | 0 | |
| control A54031 | 1 | 0 | |

### Example 4

The CP4 EPSPS gene may be used to select transformed plant material directly on media containing glyphosate. The ability to select and to identify transformed plant material depends, in most cases, on the use of a dominant selectable marker gene to enable the preferential and continued growth of the transformed tissues in the presence of a normally inhibitory substance. Antibiotic resistance and herbicide tolerance genes have been used almost exclusively as such dominant selectable marker genes in the presence of the corresponding antibiotic or herbicide. The nptll/kanamycin selection scheme is probably the most frequently used. It has been demonstrated that CP4 EPSPS is also a useful and perhaps superior selectable marker/selection scheme for producing and identifying transformed plants.

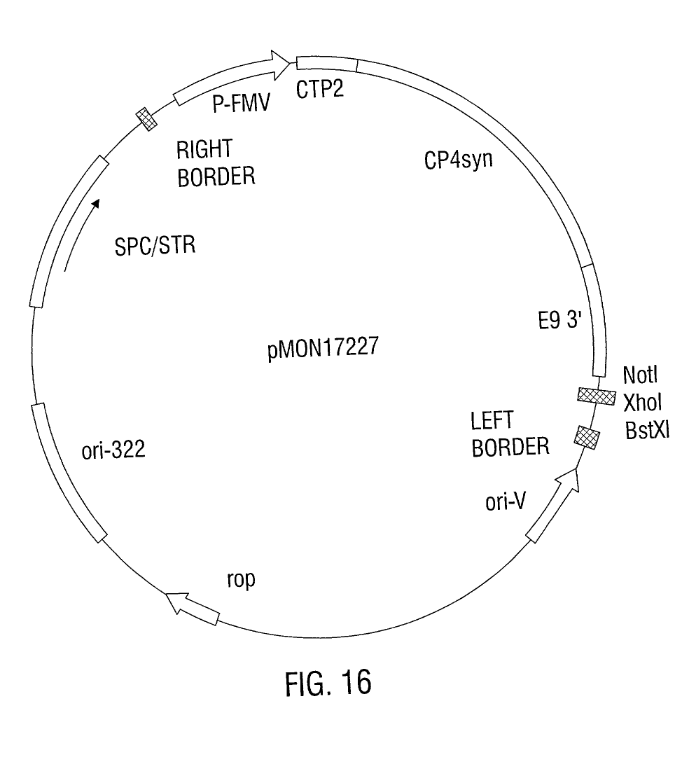

A plant transformation vector that may be used in this scheme is pMON17227 (FIG. 16). This plasmid resembles many of the other plasmids described infra and is essentially composed of the previously described bacterial replicon system that enables this plasmid to replicate in E. coli and to be introduced into and to replicate in Agrobacterium, the bacterial selectable marker gene (Spc/Str), and located between the T-DNA right border and left border is the CTP2-CP4 synthetic gene in the FMV35S promoter-E9 3′cassette. This plasmid also has single sites for a number of restriction enzymes, located within the borders and outside of the expression cassette. This makes it possible to easily add other genes and genetic elements to the vector for introduction into plants.

The protocol for direct selection of transformed plants on glyphosate is outlined for tobacco. Explants are prepared for pre-culture as in the standard procedure as described in Example 1: surface sterilization of leaves from 1 month old tobacco plants (15 minutes in 10% clorox+surfactant; 3X dH₂O washes); explants are cut in 0.5×0.5 cm squares, removing leaf edges, mid-rib, tip, and petiole end for uniform tissue type; explants are placed in single layer, upside down, on MS104 plates+2 ml 4COO5K media to moisten surface; pre-culture 1-2 days. Explants are inoculated using overnight culture of Agrobacterium containing the plant transformation plasmid that is adjusted to a titer of 1.2 X 109 bacteria/ml with 4C005K media. Explants are placed into a centrifuge tube. the Agrobacterium suspension is added and the mixture of bacteria and explants is “Vortexed” on maximum setting for 25 seconds to ensure even penetration of bacteria. The bacteria are poured off and the explants are blotted between layers of dry sterile filter paper to remove excess bacteria. The blotted explants are placed upside down on MS104 plates+2 ml 4COO5K media+filter disc. Co-culture is 2-3 days. The explants are transferred to MS104+Carbenicillin 1000 mg/l +cefotaxime 100 mg/l for 3 days (delayed pbase). The explants are then transferred to MS104+glyphosate 0.05 mM +Carbenicillin 1000 mg/ +cefotaxime 100 mg/l for selection phase. At 4-6 weeks shoots are cut from callus and placed on MSO+Carbenicillin 500 mgll rooting media. Roots form in 3-5 days, at which time leaf pieces can be taken from rooted plates to confirm glyphosate tolerance and that the material is transformed.

The presence of the CP4 EPSPS protein in these transformed tissues has been confirmed by immunoblot analysis of leaf discs. The data from one experiment with pMON17227 is presented in the following: 139 shoots formed on glyphosate from 400 explants inoculated with Agrobacterium ABI/pMON17227; 97 of these were positive on recallusing on glyphosate. These data indicate a transformation rate of 24 per 100 explants, which makes this a highly efficient and time saving transformation procedure for plants. Similar transformation frequencies have been obtained with pMON17131 and direct selection of transformants on glyphosate with the CP4 EPSPS genes has also been shown in other plant species, including, Arabidopsis, soybean, corn, wheat, potato, tomato, cotton, lettuce, and sugarbeet.

The pMON17227 plasmid contains single restriction enzyme recognition cleavage sites (NotI, XhoI, and BstX₁ ) between the CP4 glyphosate selection region and the left border of the vector for the cloning of additional genes and to facilitate the introduction of these genes into plants.

### Example 5A

The CP4 EPSPS gene has also been introduced into Black Mexican Sweet (BMS) corn cells with expression of the protein and glyphosate resistance detected in callus.

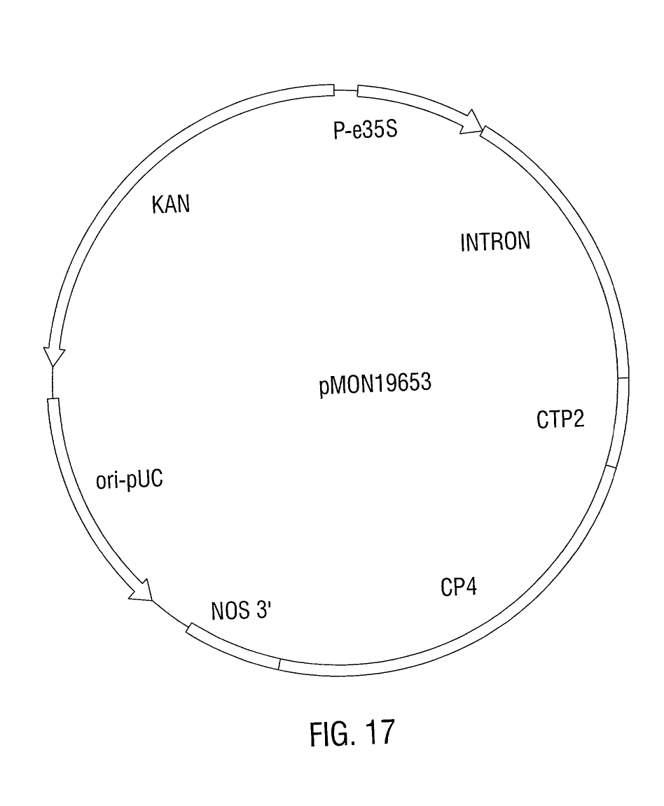

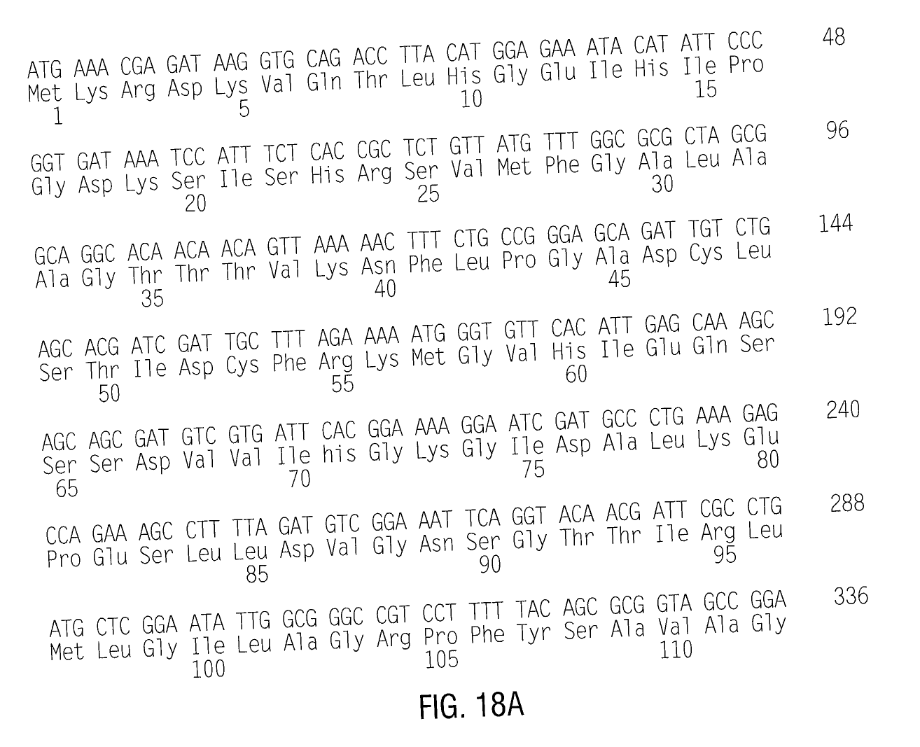

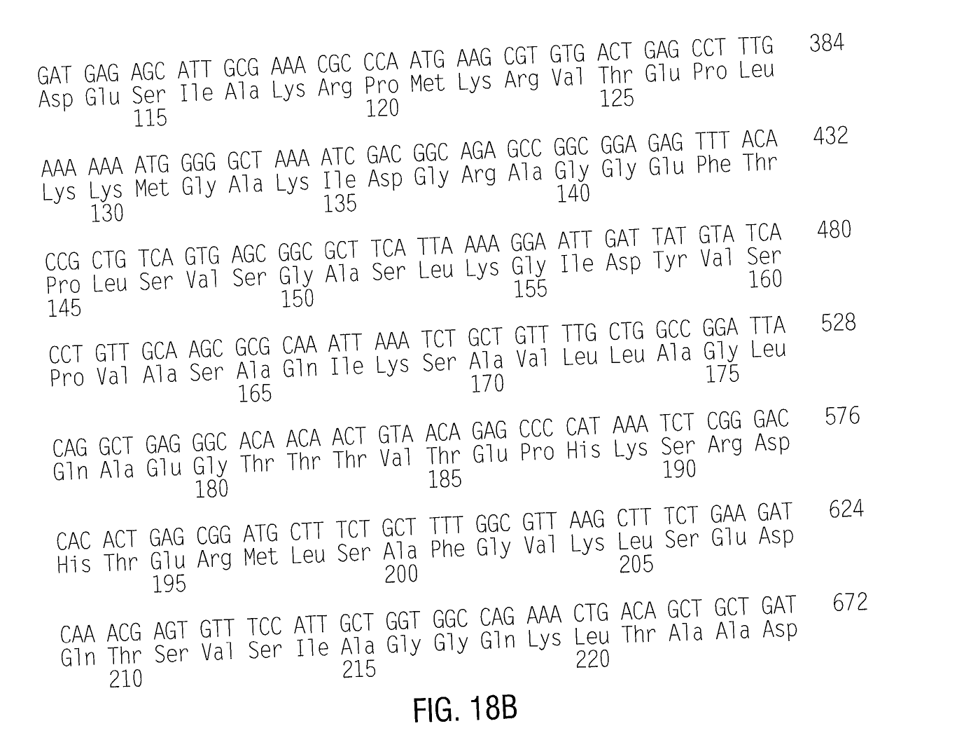

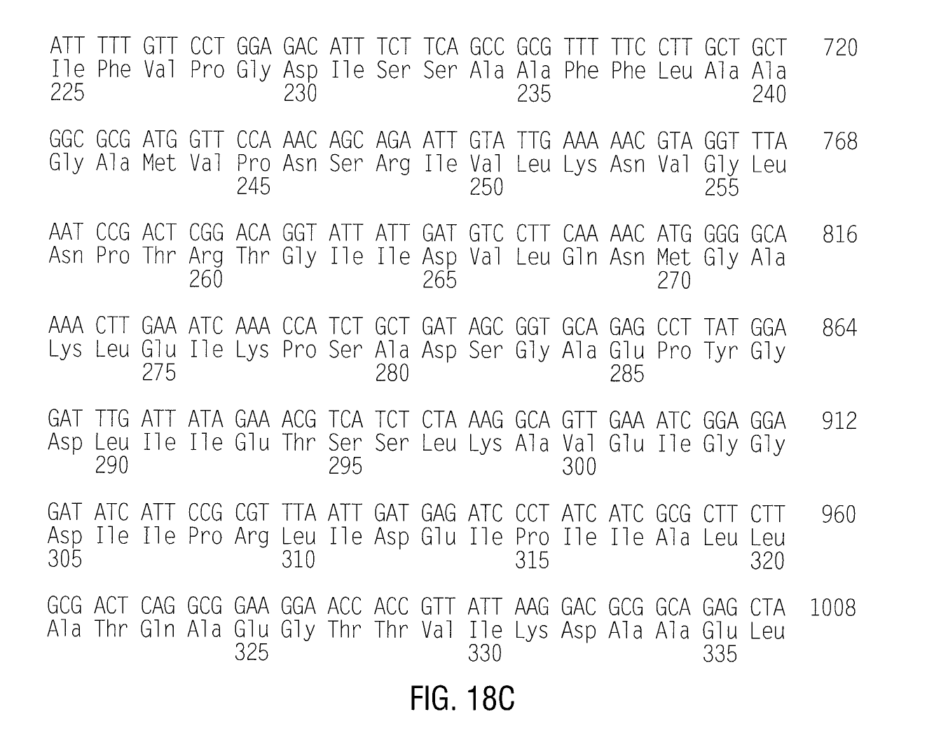

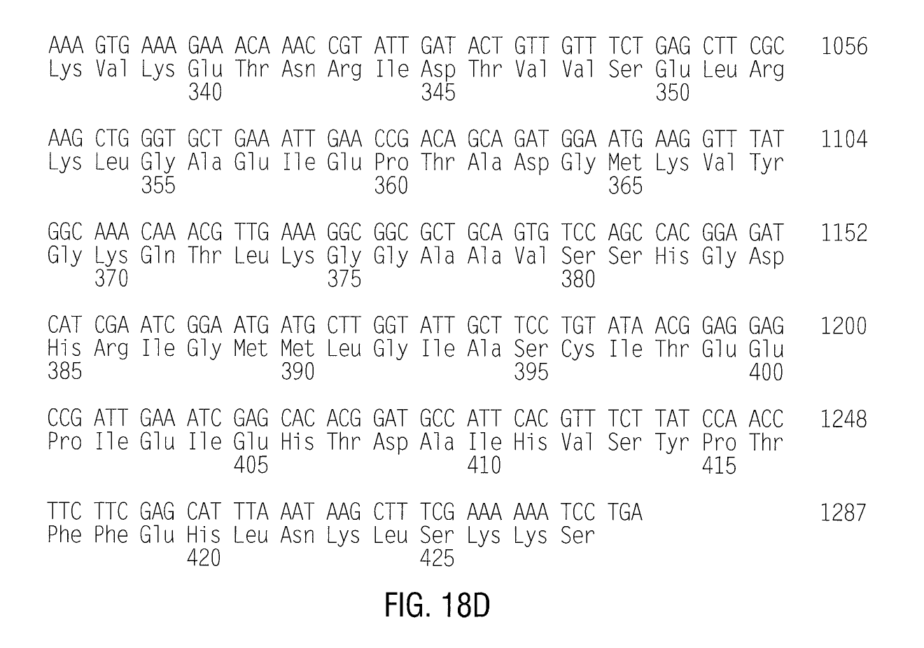

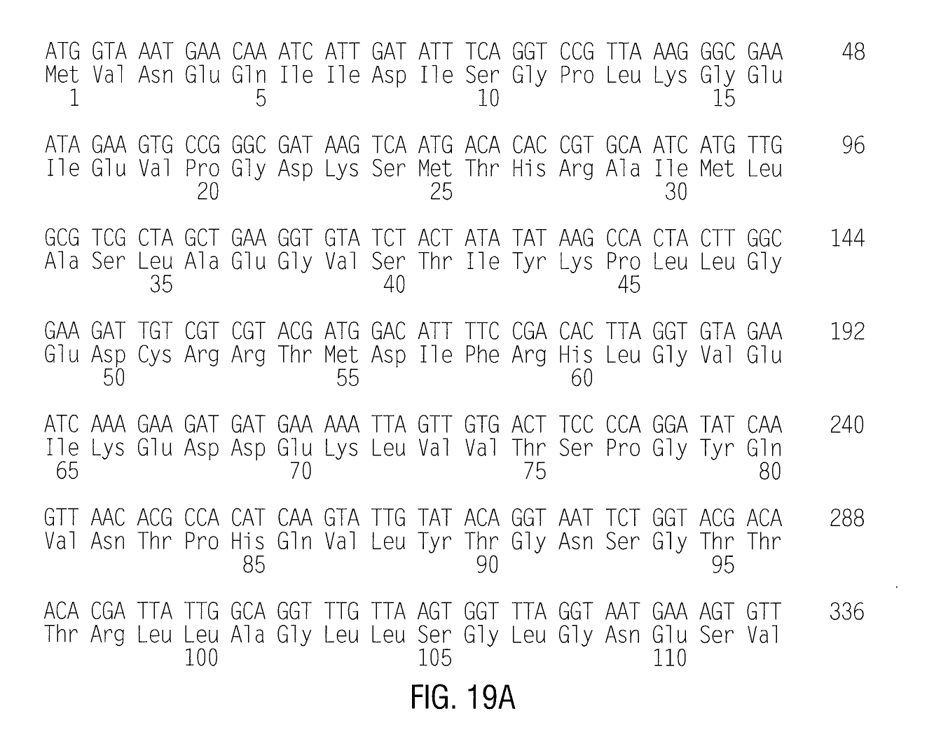

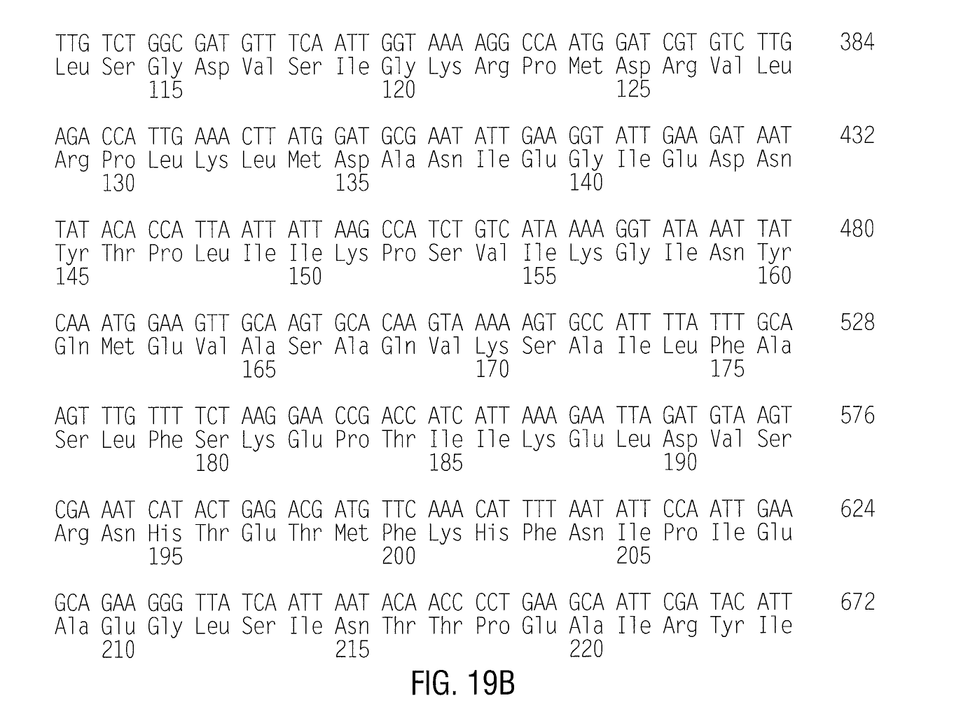

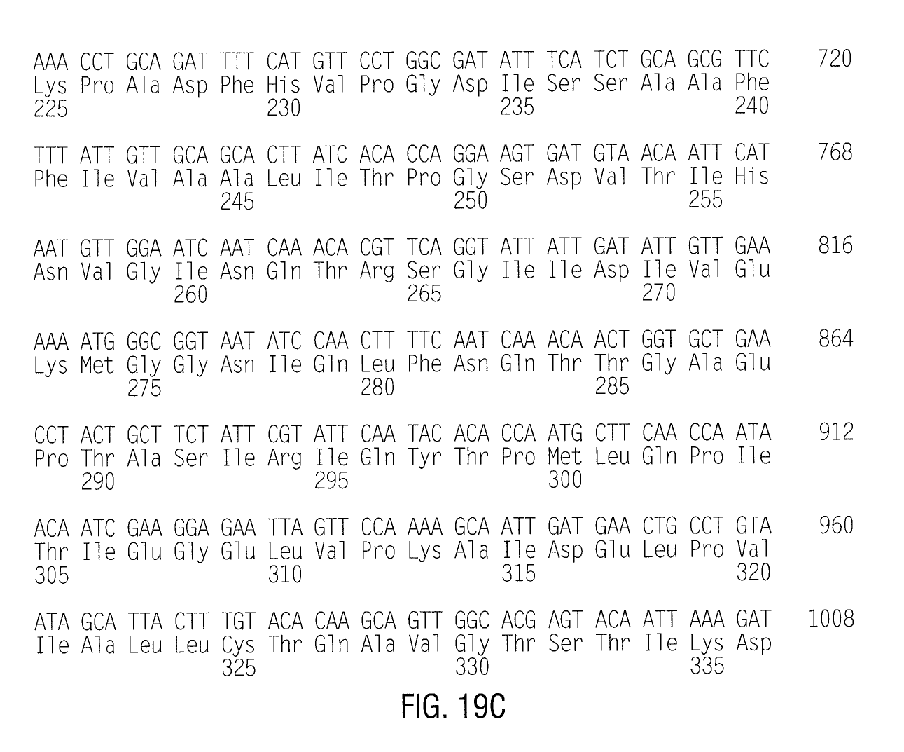

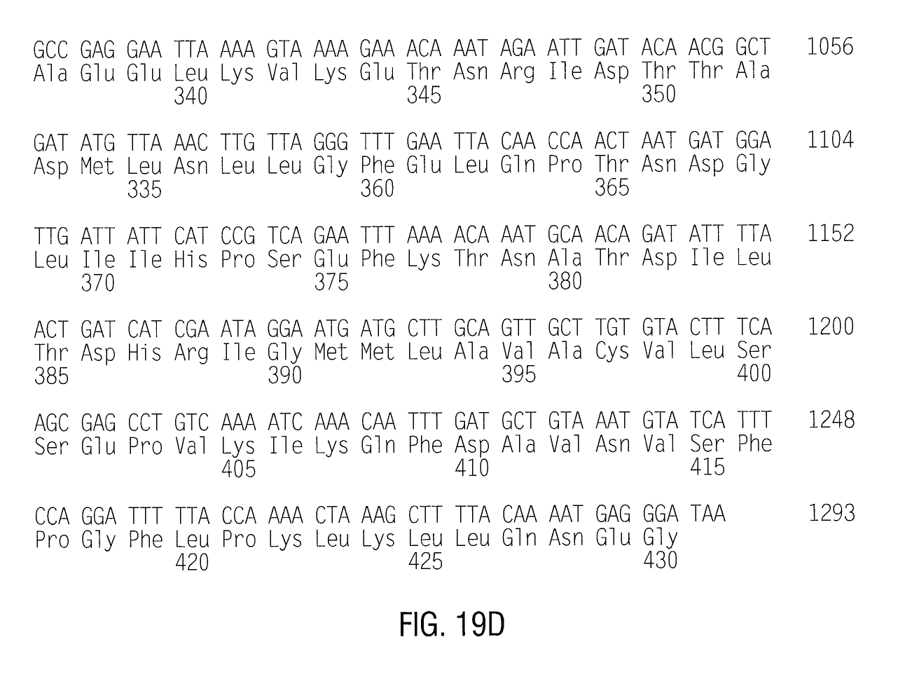

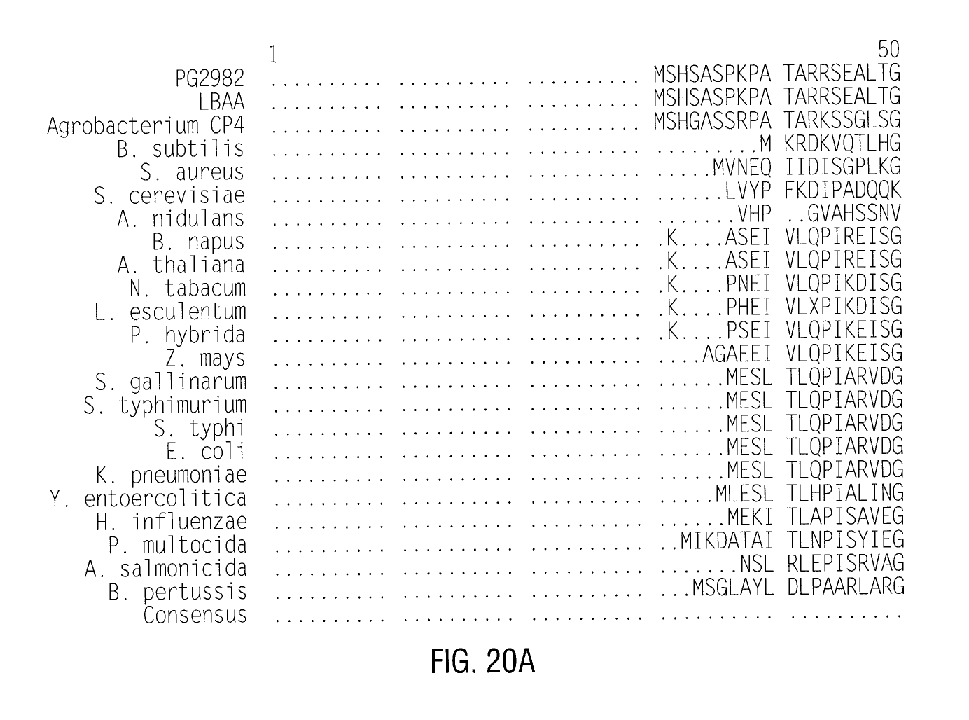

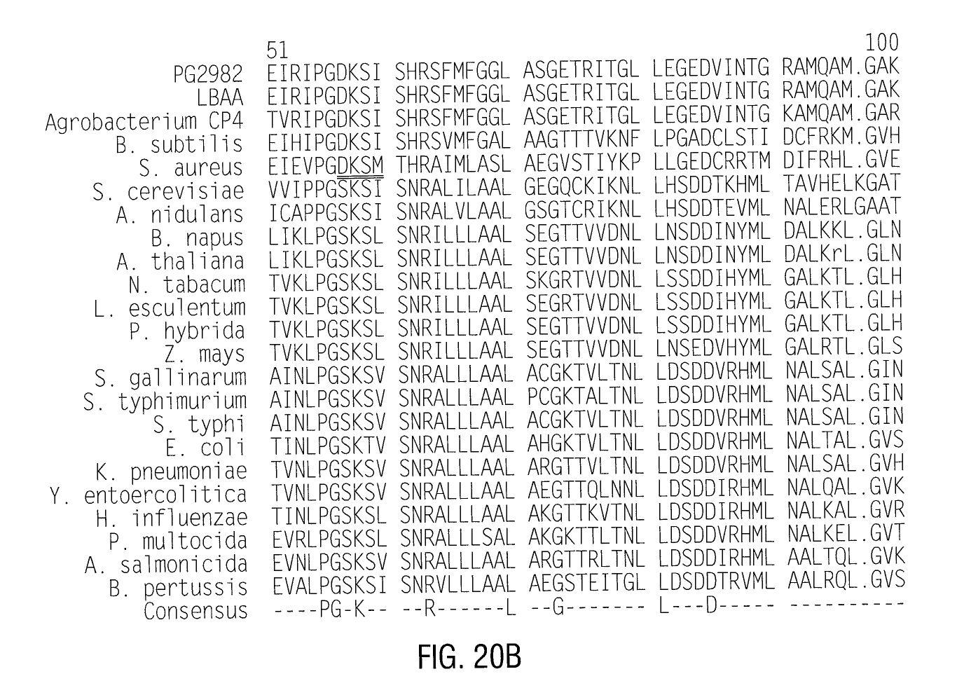

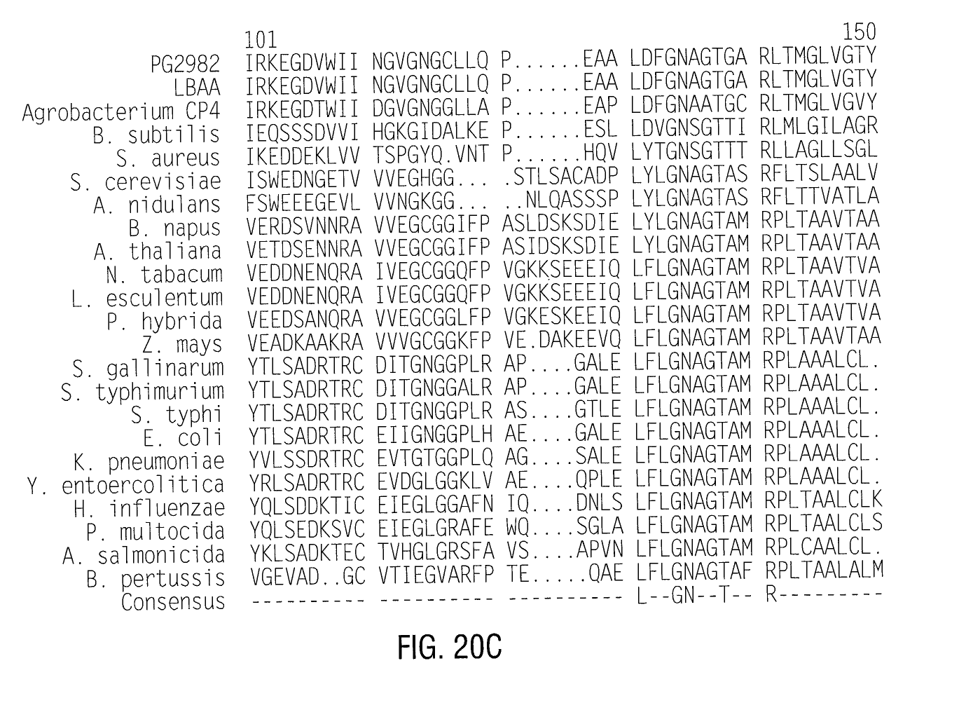

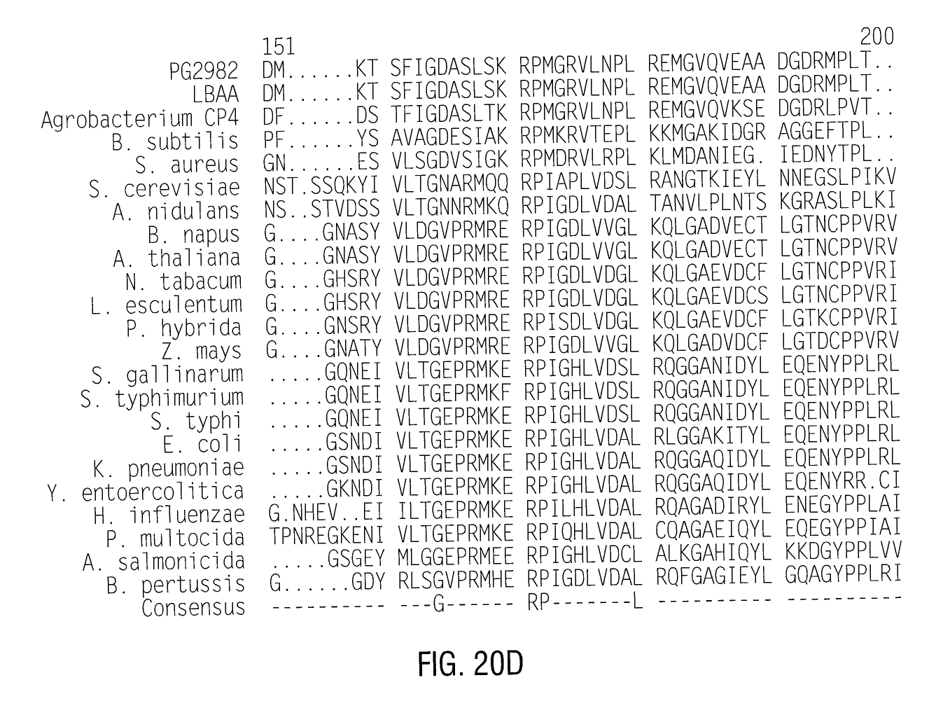

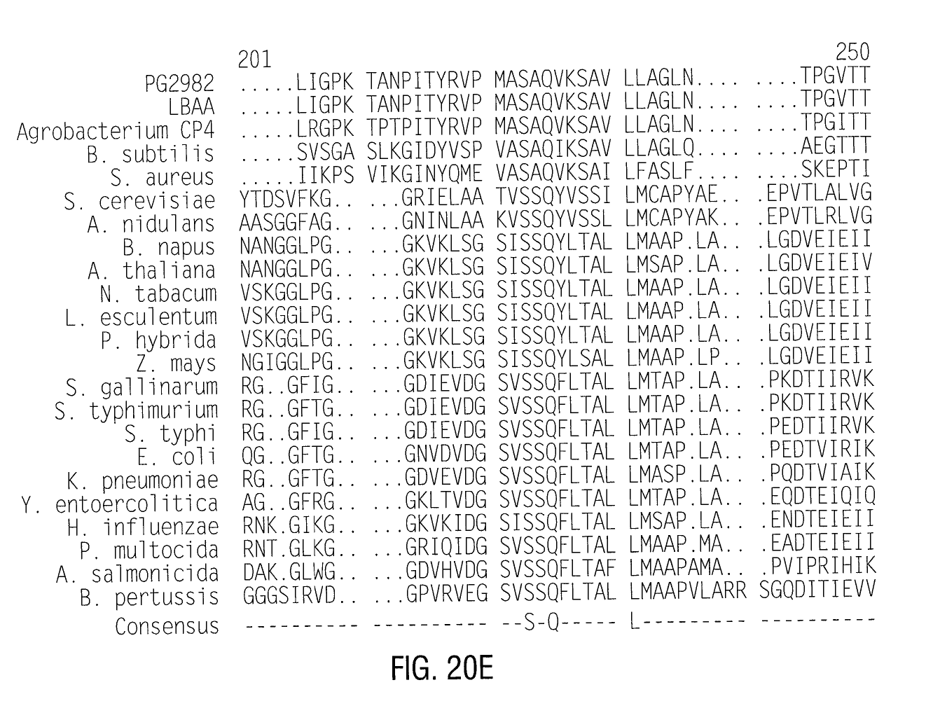

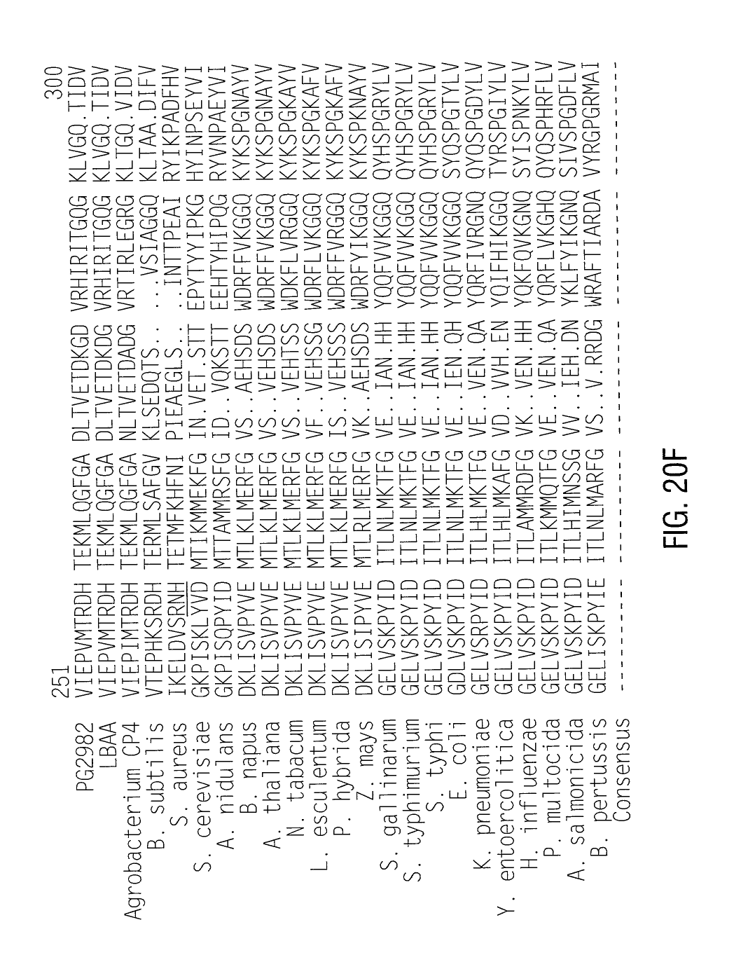

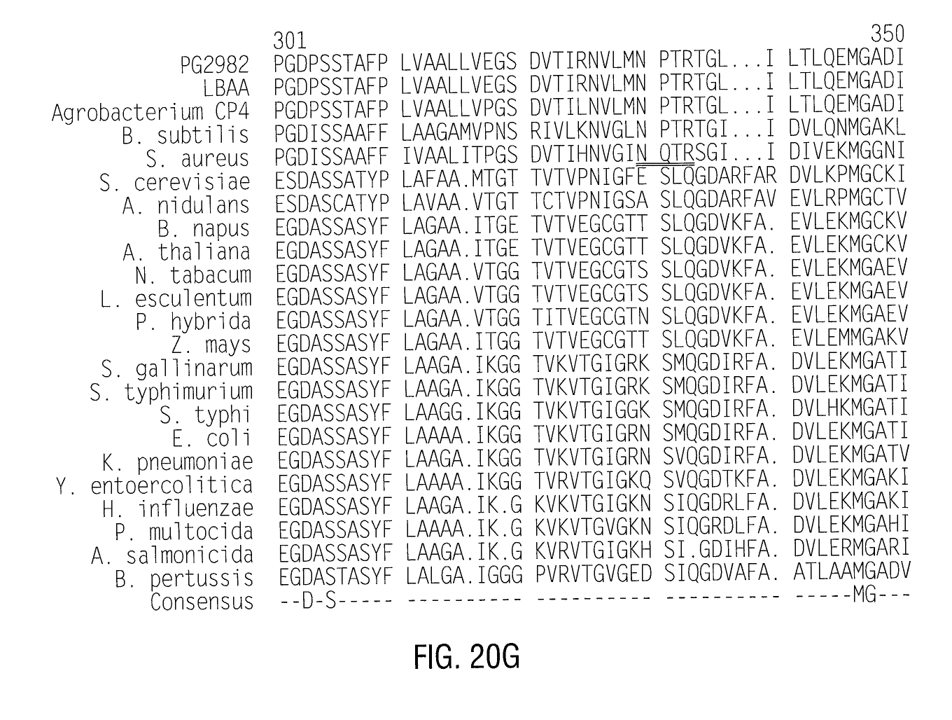

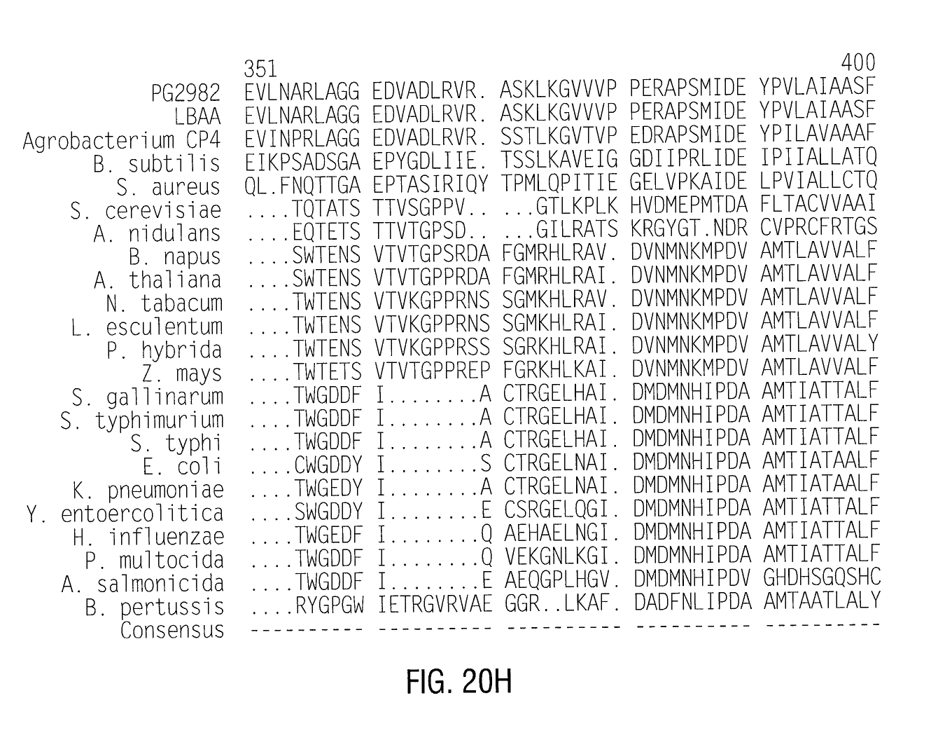

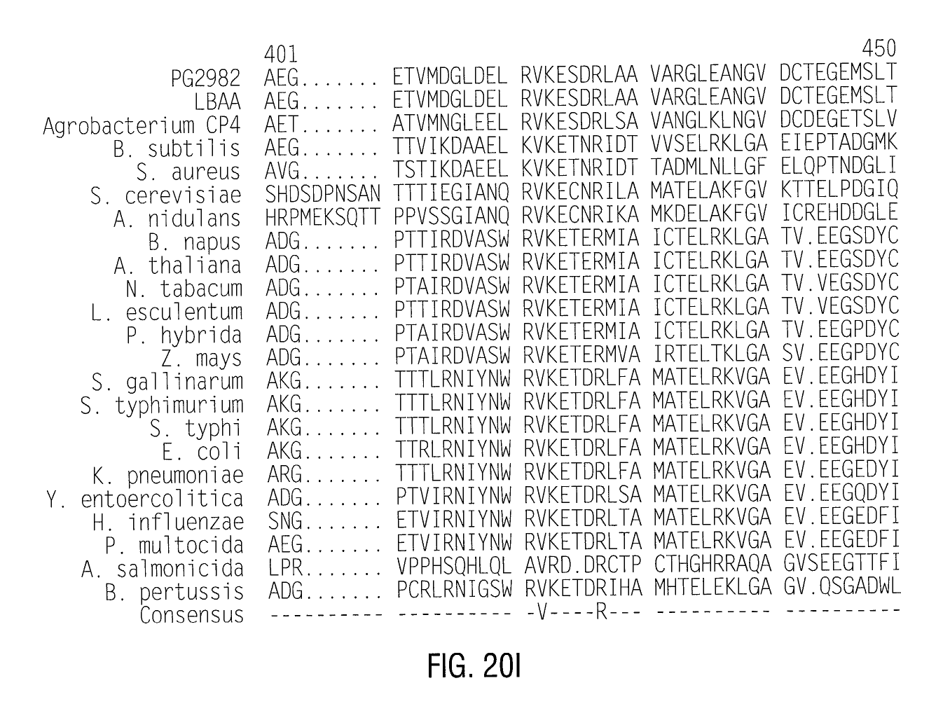

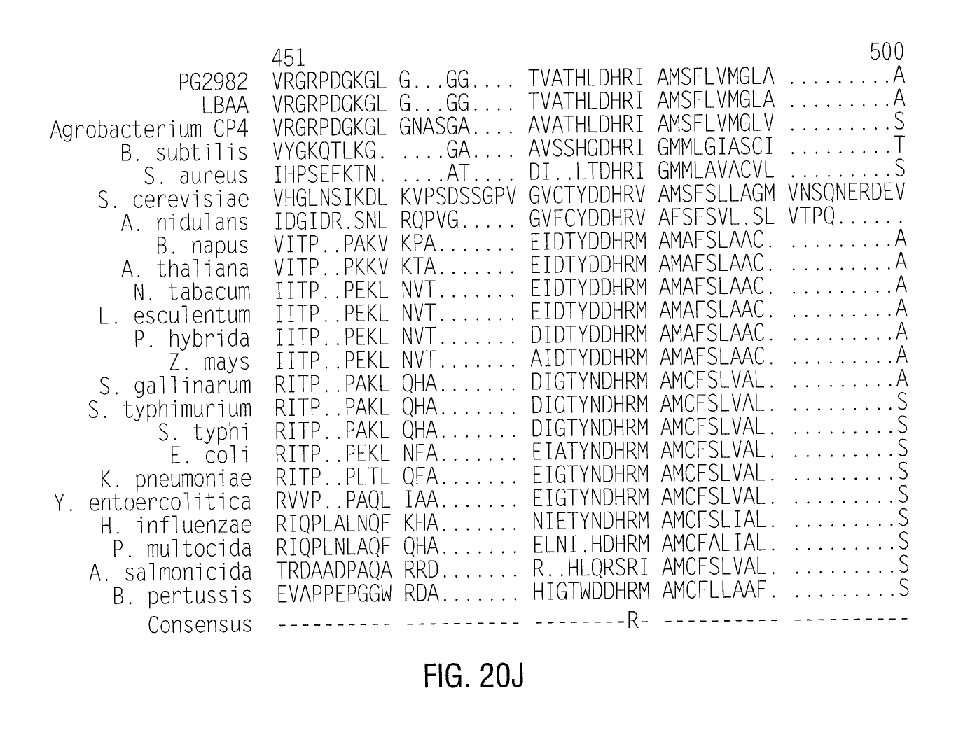

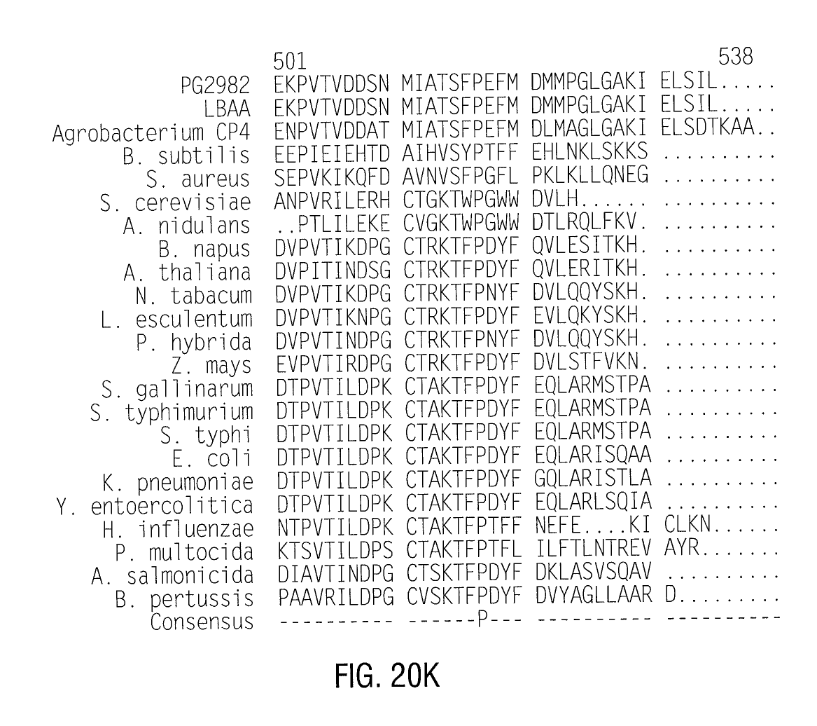

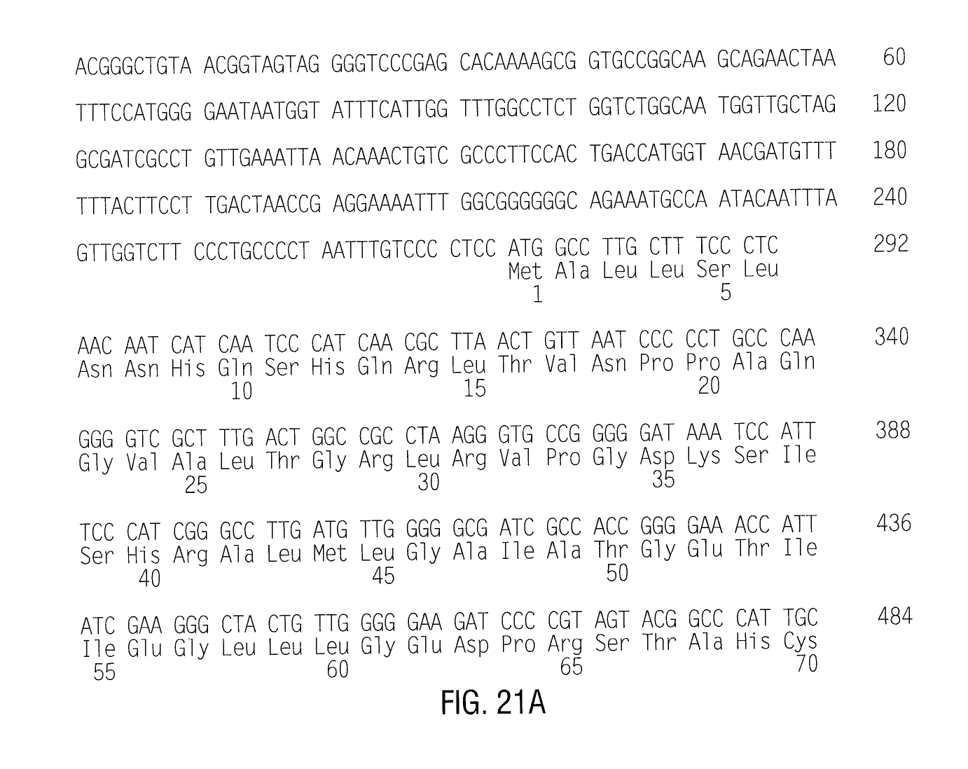

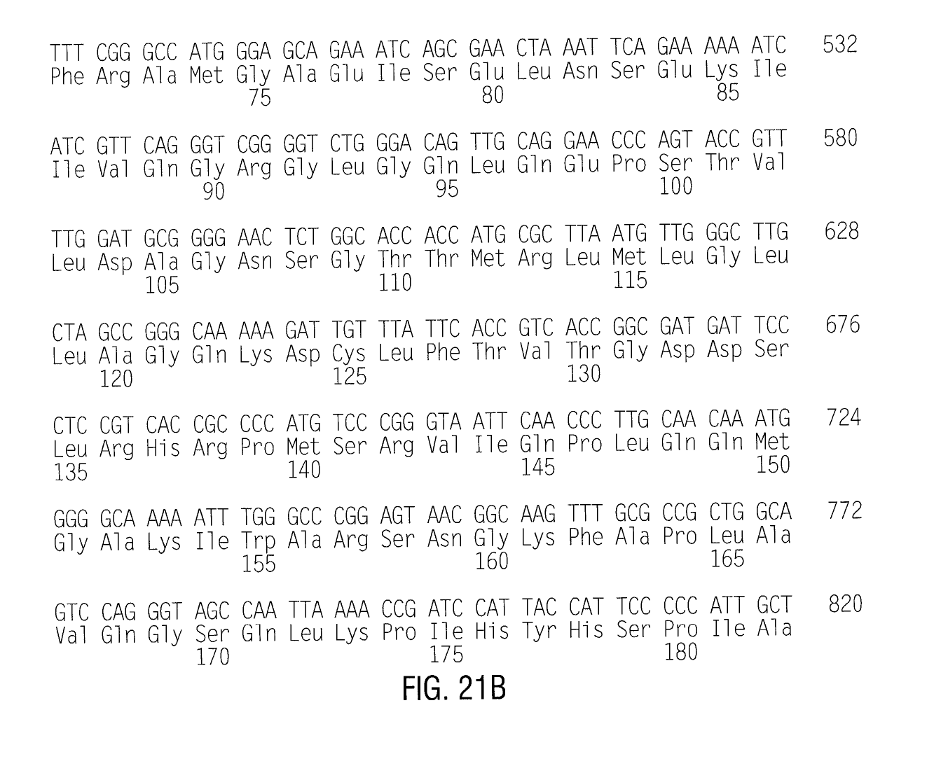

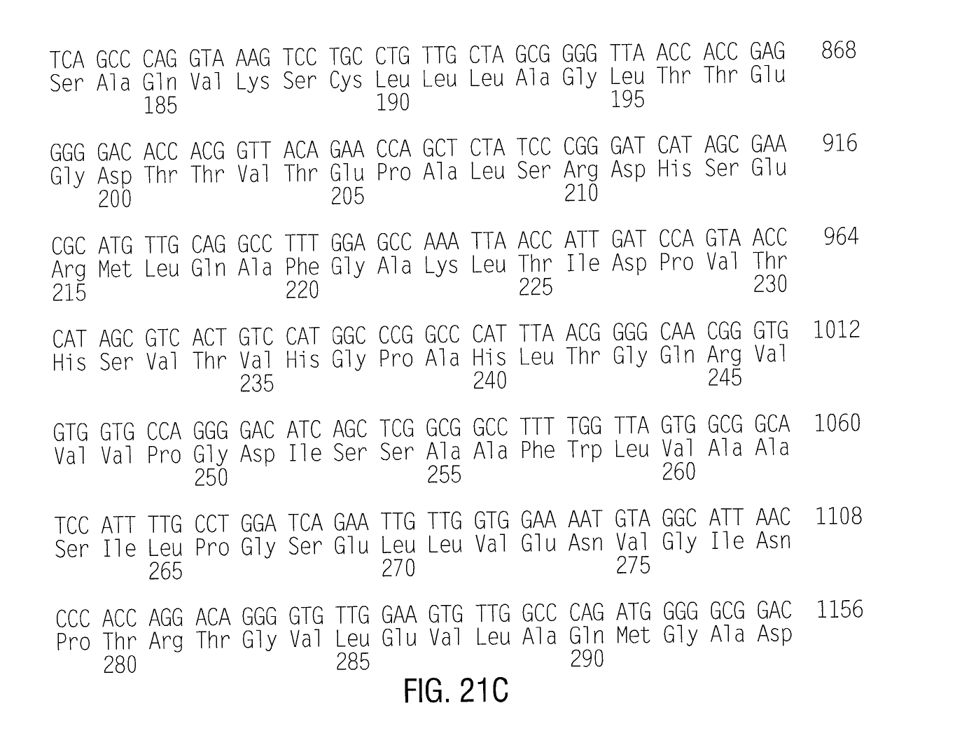

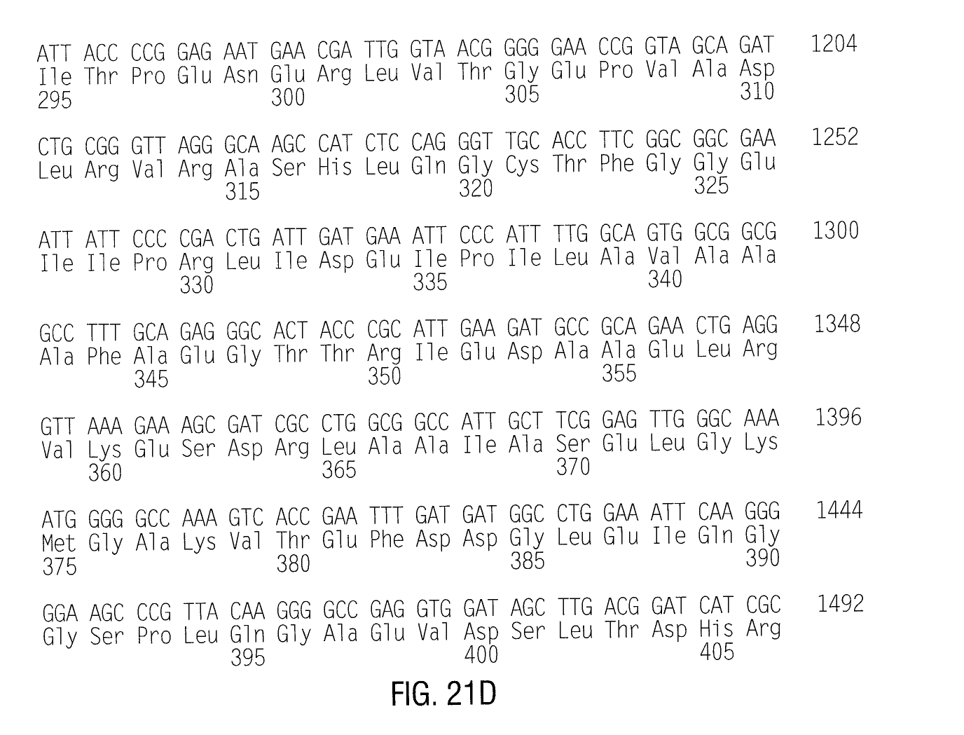

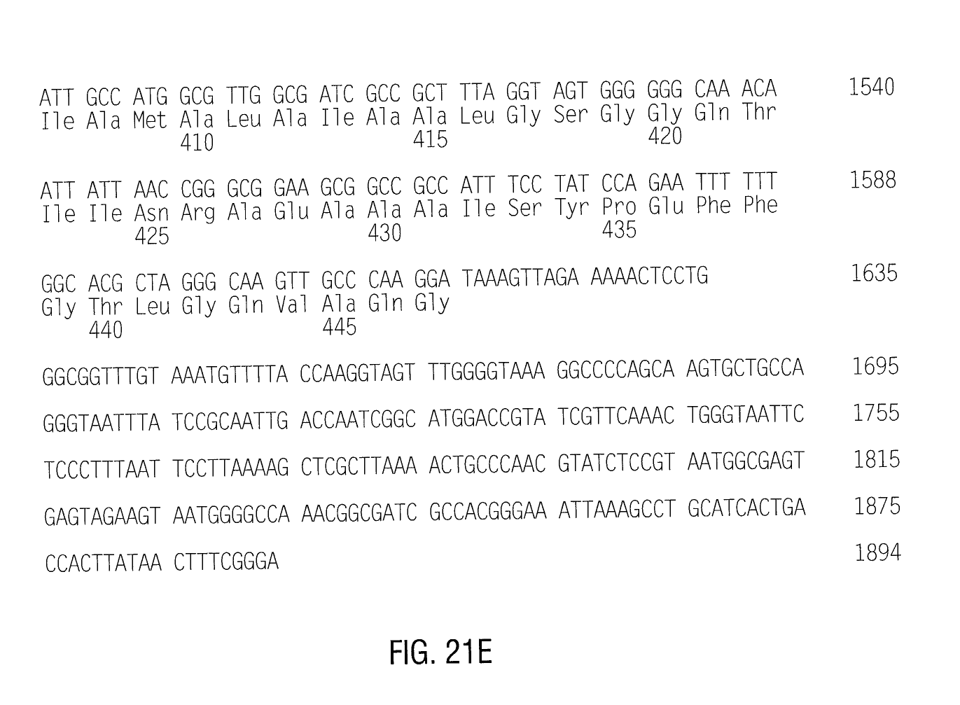

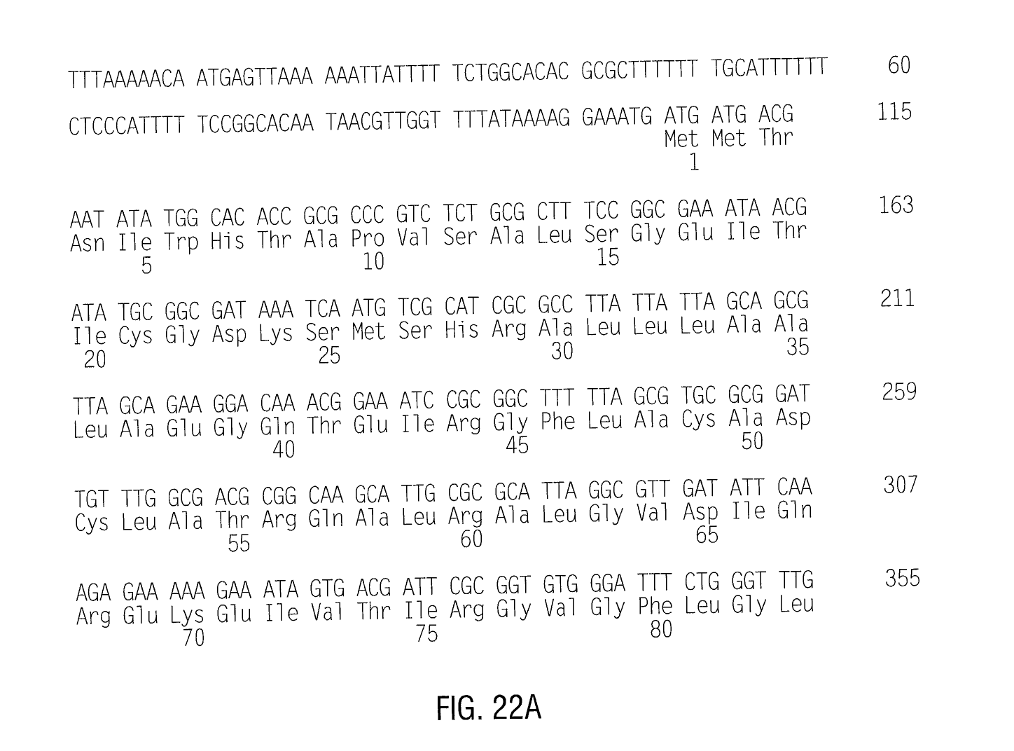

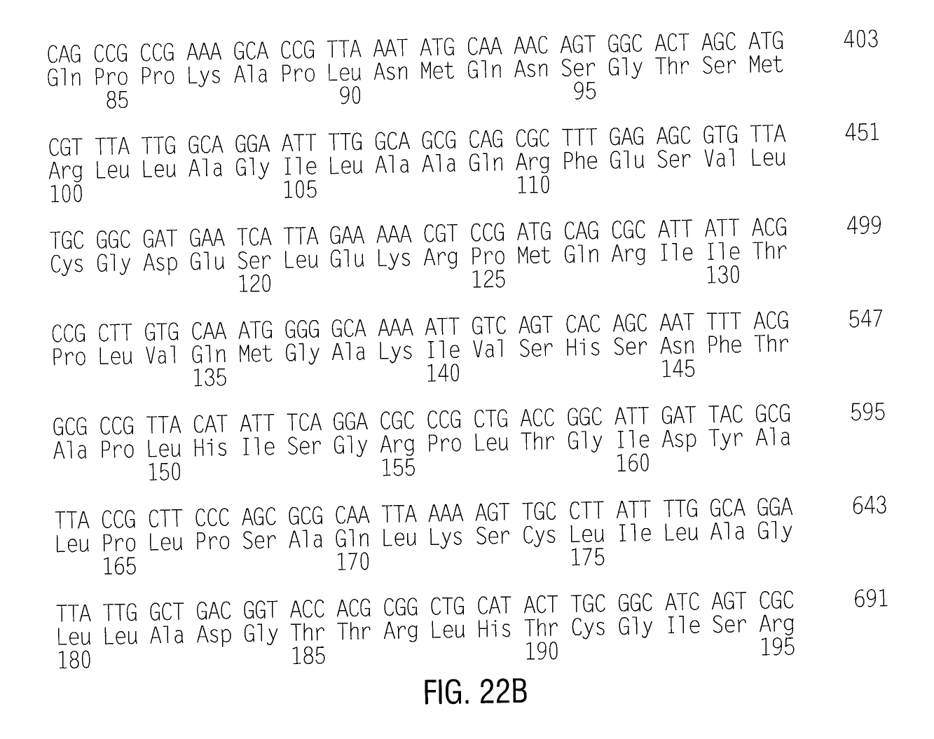

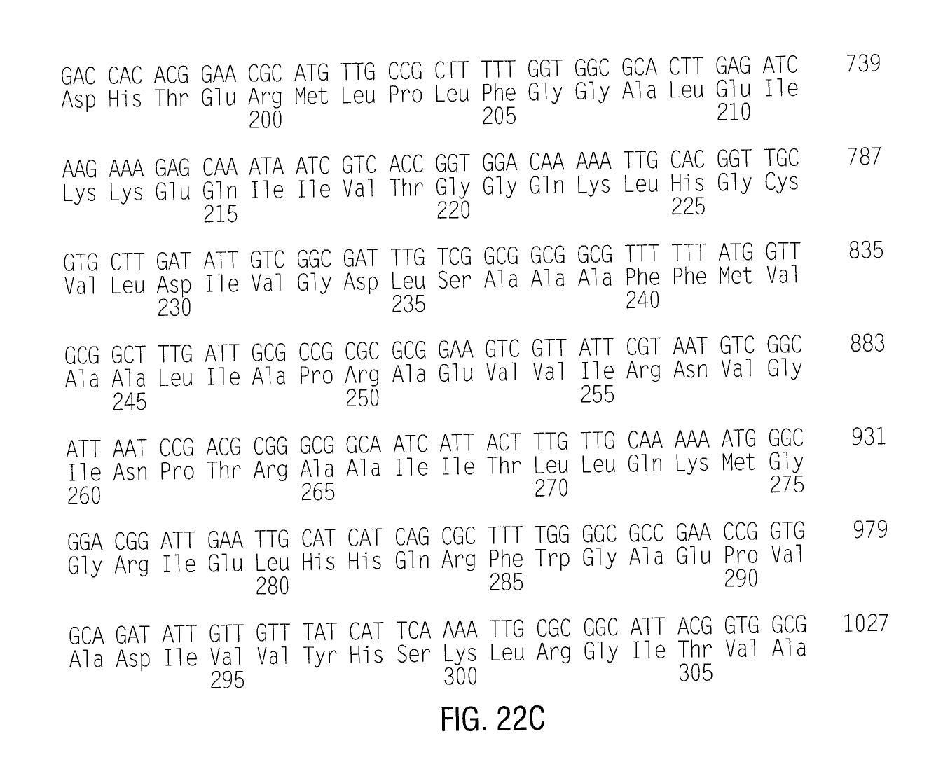

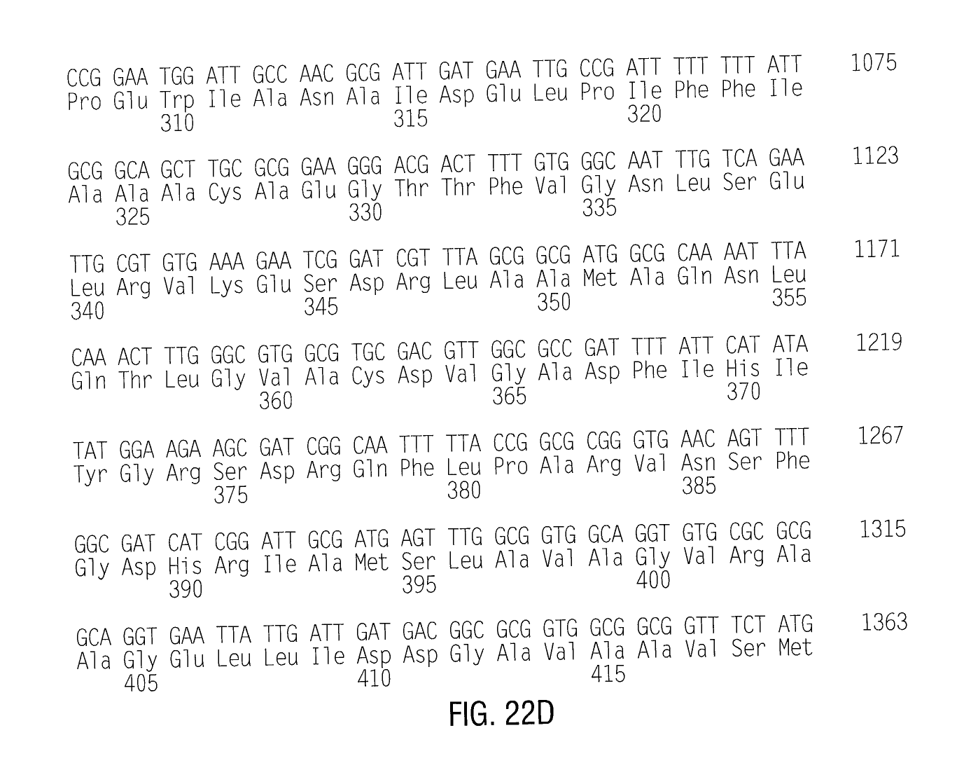

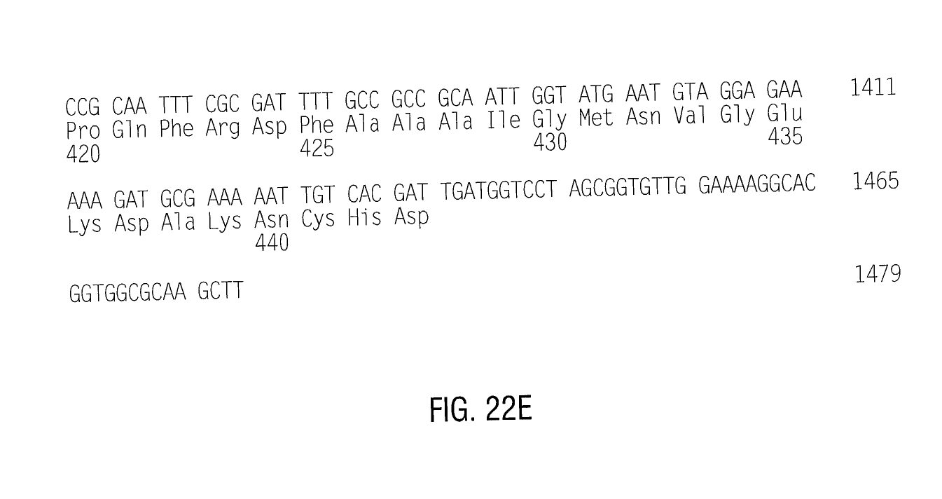

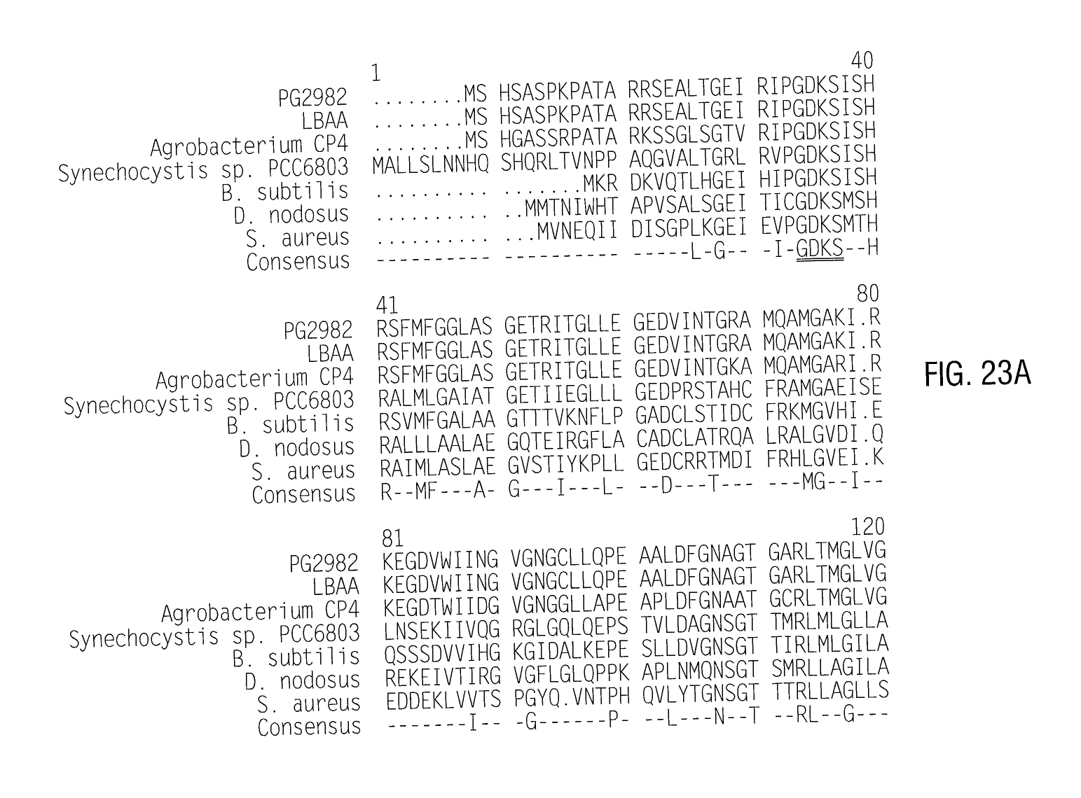

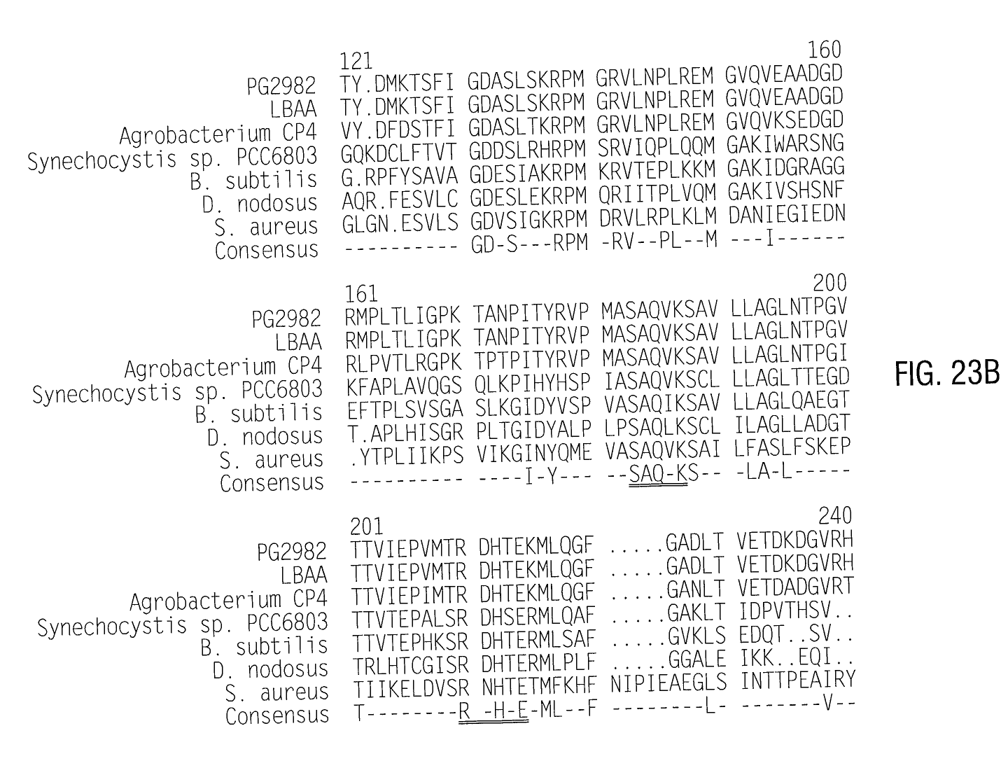

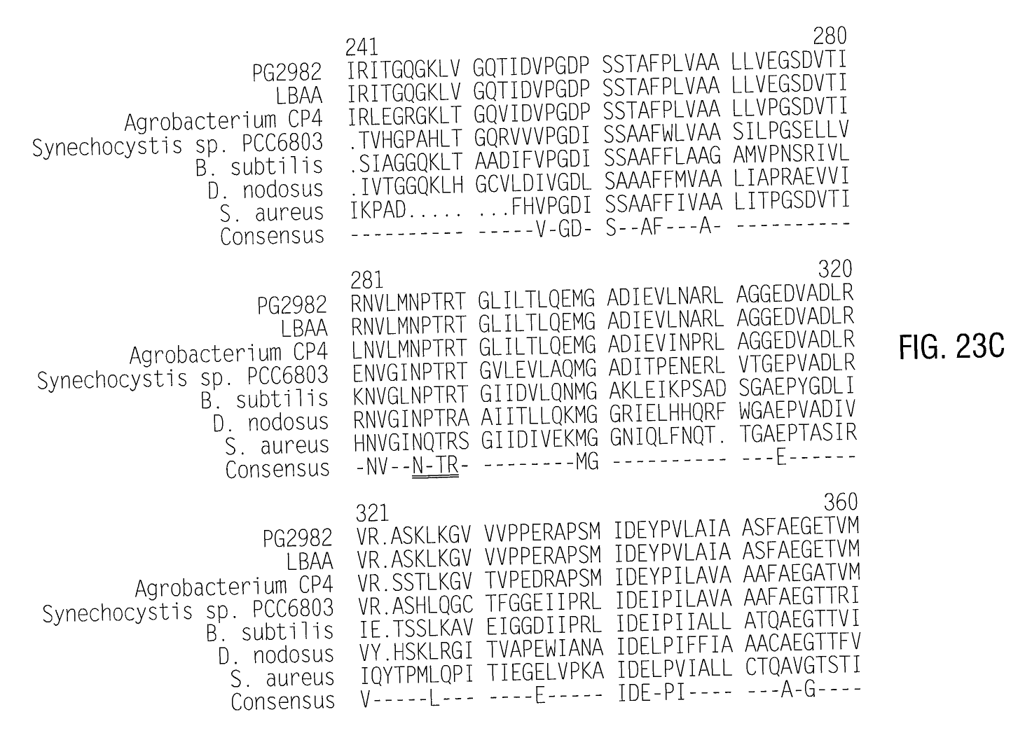

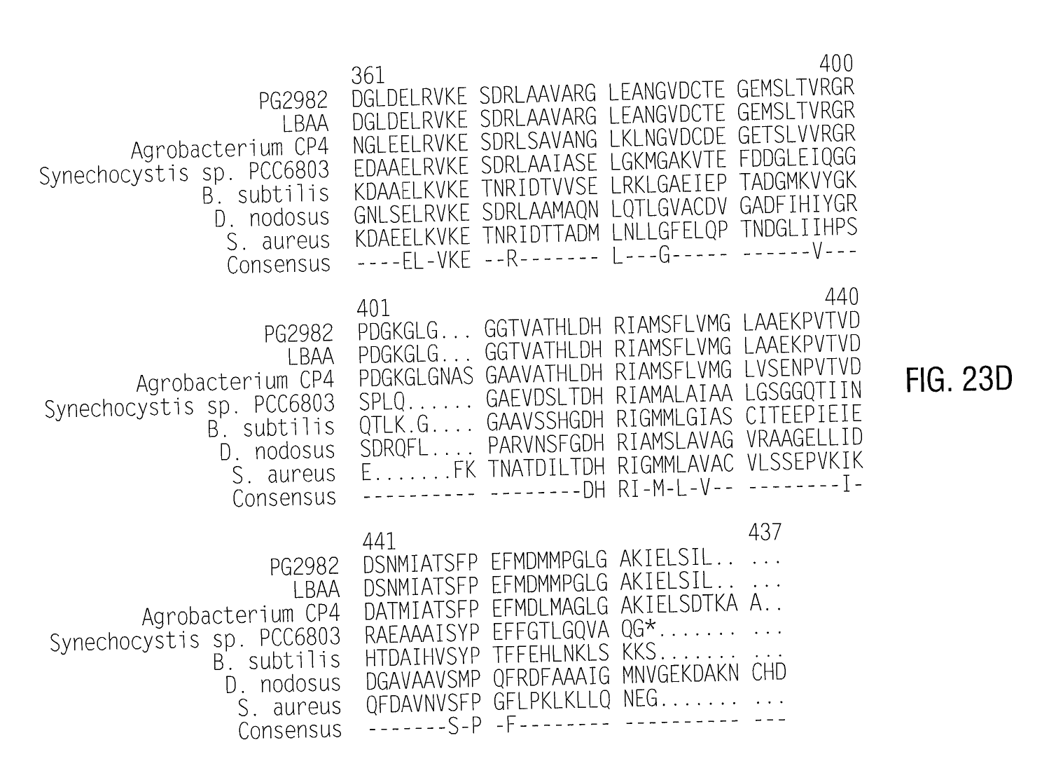

The backbone for this plasmid was a derivative of the high copy plasmid pUC119 (Viera and Messing, 1987). The 1.3 Kb FspI-DraI pUC119 fragment containing the origin of replication was fused to the 1.3 Kb Smal-HindIH filled fragment from pKC7 (Rao and Rogers, 1979) which contains the neomycin phosphotransferase type II gene to confer bacterial kanamycin resistance. This plasmid was used to construct a monocot expression cassette vector containing the 0.6 kb cauliflower mosaic virus (CaMV) 35S RNA promoter with a duplication of the -90 to -300 region (Kay et al., 1987), an 0.8 kb fragment containing an intron from a maize gene in the 5′ untranslated leader region, followed by a polylinker and the 3′ termination sequences from the nopaline synthase (NOS) gene (Fraley et al., 1983). A 1.7 Kb fragment containing the 300 bp chloroplast transit peptide from the Arabidopsis EPSP synthase fused in frame to the 1.4 Kb coding sequence for the bacterial CP4 EPSP synthase was inserted into the monocot expression cassette in the polylinker between the intron and the NOS termination sequence to form the plasmid pMON19653 (FIG. 17).

pMON19653 DNA was introduced into Black Mexican Sweet (BMS) cells by co-bombardment with EC9, a plasmid containing a sulfonylurea-resistant form of the maize acetolactate synthase gene. 2.5 mg of each plasmid was coated onto tungsten particles and introduced into log-phase BMS cells using a PDS-1000 particle gun essentially as described (Klein et al., 1989). Transformants are selected on MS medium containing 20 ppb chlorsulfuron. After initial selection on chlorsulfuron, the calli can be assayed directly by Western blot. Glyphosate tolerance can be assessed by transferring the calli to medium containing 5 mM glyphosate. As shown in Table XI, CP4 EPSPS erance to corn callus.

TABLE XI
Expression of CP4 in BMS Corn Callus - pMON 19653

| | CP expression |
|---|---|
| Line | (% extracted protein) |
| 284 | 0.006% |
| 287 | 0.036 |
| 290 | 0.061 |
| 295 | 0.073 |
| 299 | 0.113 |
| 309 | 0.042 |
| 313 | 0.003 |

To measure CP4 EPSPS expression in corn callus, the following procedure was used: BMS callus (3 g wet weight) was dried on filter paper (Whatman#1) under vacuum, reweighed, and extraction buffer (500 μl/g dry weight; 100 mM Tris, 1 mm EDTA, 10% glycerol) was added. The tissue was homogenized with a Wheaton overhead stirrer for 30 seconds at 2.8 power setting. After centrifugation (3 minutes, Eppendorf microfuge), the supernatant was removed and the protein was quantitated (BioRad Protein Assay). Samples (50 μg/well) were loaded on an SDS PAGE gel (Jule, 3-17%) along with CP4 EPSPS standard (10 ng), electrophoresed, and transferred to nitrocellulose similarly to a previously described method (Padgette, 1987). The nitrocellulose blot was probed with goat anti-CP4 EPSPS IgG, and developed with I-125 Protein G. The radioactive blot was visualized by autoradiography. Results were quantitated by densitometry on an LKB UltraScan XL laser densitomer and are tabulated below in Table X.

TABLE XII
Glyphosate resistance in BMS Corn Callus
using pMON 19653

| | | # chlorsulfuron- | # cross-resistant |
|---|---|---|---|
| Vector | Experiment | resistant lines | to Glyphosate |
| 19653 | 253 | 120 | 81/120 = 67.5% |
| 19653 | 254 | 80 | 37/80 = 46% |
| EC9 control | 253/254 | 8 | 0/8 = 0% |

Improvements in the expression of Class II EPSPS could also be achieved by expressing the gene using stronger plant promoters, using better 3′ polyadenylation signal sequences, optimizing the sequences around the initiation codon for ribosome loading and translation initiation, or by combination of these or other expression or regulatory sequences or factors.

### Example 5B

The plant-expressible genes encoding the CP4 EPSPS and a glyphosate oxidoreductasease enzyme (PCT Pub. No. W092/00377) were introduced into embryogenic corn callus through particle bombardment. Plasmid DNA was prepared using standard procedures (Ausubel et al., 1987), cesium-chloride purified, and re-suspended at 1 mg/ml in TE buffer. DNA was precipitated onto M10 tungsten or 1.0 μgold particles (BioRad) using a calcium chloride/spermidine precipitation protocol, essentially as described by Klein et al. (1987). The PDS1000® gunpowder gun (BioRad) was used. Callus tissue was obtained by isolating 1-2 mm long immature embryos from the “Hi-II” genotype (Armstrong et al., 1991), or Hi-II X B73 crosses, onto a modified N6 medium (Armstrong and Green, 1985; Songstad et al., 1991). Embryogenic callus (“type-II”; Armstrong and Green, 1985) initiated from these embryos was maintained by subculturing at two week intervals, and was bombarded when less than two months old. Each plate of callus tissue was bombarded from 1 to 3 times with either tungsten or gold particles coated with the plasmid DNA(s) of interest. Callus was transferred to a modified N6 medium containing an appropriate selective agent (either glyphosate, or one or more of the antibiotics kanamycin, G418, or paromomycin) 1-8 days following bombardment, and then re-transferred to fresh selection media at 2-3 week intervals. Glyphosate-resistant calli first appeared approximately 6-12 weeks post-bombardment. These resistant calli were propagated on selection medium, and samples were taken for assays gene expression. Plant regeneration from resistant calli was accomplished essentially as described by Petersen et al. (1992).

In some cases, both gene(s) were covalently linked together on the same plasmid DNA molecule. In other instances, the genes were present on separate plasmids, but were introduced into the same plant through a process termed “co-transformation”. The 1 mg/ml plasmid preparations of interest were mixed together in an equal ratio, by volume, and then precipitated onto the tungsten or gold particles. At a high frequency, as described in the literature (e.g., Schocher et al., 1986), the different plasmid molecules integrate into the genome of the same plant cell. Generally the integration is into the same chromosomal location in the plant cell, presumably due to recombination of the plasmids prior to integration. Less frequently, the different plasmids integrate into separate chromosomal locations. In either case, there is integration of both DNA molecules into the same plant cell, and any plants produced from that cell.

Transgenic corn plants were produced as decribed above which contained a plant-expressible CP4 gene and a plant-expressible gene encoding a glyphosate oxidoreductase enzyme.

The plant-expressible CP4 gene comprised a structural DNA sequence encoding a CTP2/CP4 EPSPS fusion protein. The CTP2/CP4 EPSPS is a gene fusion composed of the N-terminal 0.23 Kb chloroplast transit peptide sequence from the Arabidopsis thaiana EPSPS gene (KIlee et al. 1987, referred to herein as CTP2), and the C-terminal 1.36 Kb 5-enolpyruvylshikimate-3-phosphate synthase gene (CP4) from an Agrobacterium species. Plant expression of the gene fusion produces a pre-protein which is rapidly imported into chloroplasts where the CTP is cleaved and degraded (della-Cioppa et al., 1986) releasing the mature CP4 protein.

The plant-expressible gene expressing a glyphosate oxidoreductase enzyme comprised a structual DNA sequence comprising CTPIJGOXsyn gene fusion composed of the N-terminal 0.26 Kb chloroplast transit peptide sequence derived from the Arabidopsis thaliana SSU la gene (Timko et al., 1988 referred to herein as CTP1), and the C-terminal 1.3 Kb synthetic gene sequence encoding a glyphosate oxidoreductase enzyme (GOXsyn, as descibed in PCT Pub. No. W092100377 previously incorporated by reference). The GOXsyn gene encodes the enzyme glyphosate oxidoreductase from an Achromobacter sp. strain LBAA which catalyzes the conversion of glyphosate to herbicidally inactive products, aminomethylphosphonate and glyoxylate. Plant expression of the gene fusion produces a pre-protein which is rapidly imported into chloroplasts where the CTP is cleaved and degraded (della- Cioppa et al., 1986) releasing the mature GOX protein.

Both of the above described genes also include the following regulatory sequences for plant expression: (i) a promoter region comprising a 0.6 Kb 35S cauliflower mosaic virus (CaMV) promoter (Odell et al., 1985) with the duplicated enhancer region (Kay et al., 1987) which also contains a 0.8 Kb fragment containing the first intron from the maize heat shock protein 70 gene (Shah et al., 1985 and PCT Pub. No. W093/19189, the disclosure of which is hereby incorporated by reference); and (ii) a 3′ non-translated region comprising a 0.3 Kb fragment of the 3′ non-translated region of the nopaline synthase gene (Fraley et al., 1983 and Depicker, et al. 1982) which functions to direct polyadenylation of the mRNA.

The above described transgenic corn plants exhibit tolerance to glyphosate herbicide in greenhouse and field trials.

### Example 6

The LBAA Class II EPSPS gene has been introduced into plants and also imparts glyphosate tolerance. Data on tobacco transformed with pMON17206 (infra) are presented in Table XIII.

TABLE XIII
Tobacco Glyphosate Spray Test
(pMON17206: E35S - CTP2-LBAA EPSPS: 0.4 lbs/ac)

| Line | 7 Day Rating |
|---|---|
| 33358 | 9 |
| 34586 | 9 |
| 33328 | 9 |
| 34606 | 9 |
| 33377 | 9 |
| 34611 | 10 |
| 34607 | 10 |
| 34601 | 9 |
| 34589 | 9 |
| Samsun (Control) | 4 |

From the foregoing, it will be recognized that this invention is one well adapted to attain all the ends and objects hereinabove set forth together with advantages which are obvious and which are inherent to the invention. It will be further understood that certain features and subcombinations are of utility and may be employed without reference to other features and subcombinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### BIBLIOGRAPHY

Alm, R. A., Dalrymple, B. P. and Mattick, J. S. 1994. Sequencing and expression of the aroA gene from Dichelobacter nodosus, Gene, 145: 97-101.

Alton, N. K. and Vapnek, D. (1979) Nature 282:864-869.

Ammirato, P. V., et al. Handbook of Plant Cell Culture-Crop Species. Macmillan Publ. Co. (1984).

Armstrong, C. L., and Green, C. E. 1985. Establishment and maintenance of friable, embryogenic maize callus and the involvement of L-proline. Planta 164:207-214.

Armstrong, C. L., Green, C. E., and Phillips, R. L. 1991. Development and availability of germplasm with high Type II culture formation response. Maize Genetics Coooeration NewsLetter 65:92-93.

Arnon, D. I. Plant Physiol. 24:1-15 (1949).

Ausubel, F. M., Brent, R., Kingston. R. E., Moore, D. D., Seidman, J. G., Smith, J. A., and Struhl, K 1987. CURRENT PROTOCOLS IN MOLECULAR BIOLOGY, John Wiley and Sons, Inc. New York.

Bachmann. B. J. et al., Microb. Rev., 44:1-56 (1980).

Barker, R. Idler. K., Thompson. D. and Kemp, J. (1983) Nucleotide sequence of the T-DNA region from the Agrobacterium tumefaciens Ti plasmid pTi15955. Plant Mol Biol 2: 335-350

Barry, G., Kishore, G., Padgette, S., Taylor, M., Kolacz, K, Weldon, M., Re D., Eichholtz., Fincher, K, and Hallas, L. (1992) Inhibitors of amino acid biosynthesis: Strategies for imparting glyphosate tolerance to crop plants. In: Biosynthesis and Molecular Regulation of Amino Acids in Plants. pp. 139-145. [Edited by Singh, B. K., Flores, H. E., and Shannon, J. C.] American Society of Plant Physiologists, Rockville, Ma.

Bartlett, S. G., Grossman, A. R., and Chua, N.H. (1982) in Methods in Chloropiast Molecular Biology pp. 1081-1091. M. Edelman, R.B., Hallick, and Chua, N. H.,eds.

Bevan, M. (1984) Nucleic Acids Res. 12 (22): 8711-8721.

Birnboim, H. C. and Doly, J. (1979) A rapid alkaline extraction procedure for screening recombinant plasmid DNA Nucl. Acids. Res. 7:1513-1525.

Bolivar, F., Rodriguez. R. L., Greene, P. J., Betlach, M. B., Heynecker, H. L., Boyer, H. W., Crosa, J. H., and Falkow, S. (1977) Construction and characterization of new cloning vehicles, II. A multi-purpose cloning system. Gene 2:95-113.

Boyer, H. W. and Rolland-Dussoix, D. (1969) A complementation analysis of the restriction and modification of DNA in Escherichia coli. J. Mol. Biol. 41:459.

Carrer, H. Hockenberry, T. N., Svab, Z., and Maliga, P. (1993) Kanamycin resistance as a selectable marker for plastid transformation in tobacco. Mol. Gen. Genet. 241: 49-56.

Christou, P., D. E. McCabe, and W. F. Swain (1988) Stable transformation of Soybean Callus by DNA-Coated Gold Particles. Plant Physiol. 87:671-674.

Coruzzi, G., Broglie, R., Edwards, C., and Chua, N.H. (1984). Tissue-specific and light-regulated expression of a pea nuclear gene encoding the small subunit of ribulose-1,5-bispyosphate carboxylase. EMBO J 3:1671.

Dalla Chiesa, M., Mayes, S. R., Maskell, D. J., Nixon, P. J. and Barber, J. 1994 An AroA homologue from Synechorystis sp. PCC6803, Gene, 144:145-146.

della-Cioppa, G., Bauer, S. C., Klein, B. K, Shah, D. M., Fraley, R. T. and Kishore G. K. (1986) Translocation of the precursor of 5-enolpyruvylshikimate-3-phosphate synthase into chloroplasts of higher plants in vitro. Proc. Natl. Acad Sci. USA 83: 6873-6877.

della-Cioppa, G., Bauer, S. C., Taylor, M. T., Rochester, D. E., Mein, B. K, Shah, D. M., Fraley, R. T. and Kᵢ shore G. M. (1987) Targeting a herbicide-resistant enzyme from Escherichia coli to chloroplasts of higher plants. Bio/Technology 5: 579-584.

Depicker, A., Stachel, S., Dhaese, P., Zambryski, P., and Goodman, H. M. 1982. Nopaline Synthase: Transcript Mapping and DNA Sequence. J. MOLEC. APPL. GENETICS 1:561-573.

Devereux, J., Haeberli, P. and Smithies, O. (1984) A comprehensive set of sequence analysis programs for the VAX.

Nucl. Acids. Res. 12:387-395.

Ditta, G., Stanfield, S., Corbin, D., and Helinski, D. R. (1980) Broad host range DNA cloning system for Gram—Negative bacteria: construction of a gene bank of Rhizobium meliloti. Proc Natl Acad Sci USA 77, 7347-7351.

Duncan, K., Edwards, R. M., Coggins, J. R. (1987) The pentafinctional aroM enzyme of Saccharomyces cerevisiae is a mosaic of monofinctional domains. Biochem. J. 246: 375-386.

Dunn, J. J. and Studier, F.W., (1983) J. Mol. Biol. 166:477-535.

Fitzgibbon, J. E. (1988) Pseudomonas sp. strain PG2982: uptake of glyphosate and cloning of a gene which confers increasedresistance to glyphosate. Ph. D. Dissertation, Louisiana State University.

Fitzgibbon, E. F. and Braymer, H. D. (1990) Cloning of a gene from Pseudomonas sp. PG2982 conferring increased glyphosate resistance Appl. Environ. Microbiol. 56: 3382-3388.

Fling, M. E. Kopf, J., and Richards, C. (1985). Nucleotide sequence of the transposon Tn7 gene encoding an aminoglycoside-modifying enzyme, 3′(9)-O-nucleotidyltransferase. Nucleic Acids Res. 13 no.19, 7095-7106.

Fraley, R. T., Rogers, S. G., Horsch, R. B., Sanders, P. R. Flick, J. S., Adams, S. P., Bittner, M. L., Brand, L.A., Fink, C. L. Fry, J. S., Galluppi, G. R., Goldberg, S. B., Hoffman, N. L., and Woo. S. C. 1983. Expression of bacterial genes in plant cells. Proc. Natl. Acad. Sci. USA 80:4803-4807.

Fraley, R. T., Rogers, S. G., Horsch, R. B., Eichholtz D. A., Flick, J. S., Fink, C. L., Hoffmann, N. L. and Sanders, P. R. (1985) The SEV system: a new disarmed Ti plasmid vector system for plant transformation. Bio/Technology 3: 629-635.

Fromm, M., (1990) UCLA Symposium on Molecular Strategies for Crop Improvement, Apr. 16-22, 1990. Keystone, Co.

Fry J., Barnason A., and Horsch R. (1987) Plant Cell Reports 6: 321-325.

Gasser, C. S., Winter, J. A., Hironaka, C. M. and Shah, D. M. (1988) Structure, expression, and evolution of the 5-enolpyruvylshikimate 3-phosphate synthase genes of petunia and tomato. J. Biol. Chem. 263: 42804289.

Gowda, S., Wu, F. C., and Shepard, R. J. (1989). Identification of promoter sequences for the major RNA transcripts of figwort mosaic and peanut chlorotic streak viruses (caulimovirus group). Journal of Cellular Biochemistry supplement 13D, 301 (Abstract).

Hallas, L. E., Hahn, E. M. and Korndorfer, C. (1988) Characterization of microbial traits associated with glyphosate biodegradation in industrial activated sludge. J. Industrial Microbiol. 3: 377-385.

Hayford, M. B., Medford, J. I., Hoffmann, N. L., Rogers, S. G. and Ilee, H. J. (1988) Development of a plant transformation selection system based on expression of genes encoding gentamicin acetvltransferases. Plant Physiol. 86: 1216-1222.

Herrera-Estrella, L., et al. (1983) Nature 303:209

Heitkamp, M. A., Hallas, L. and Adams, W. J. (1990) Biotreatment of industrial wastewater with immobilized microorganisms -Presented in Session 11, Paper S40, Society for Industrial Microbiology Annual Meeting, Orlando, Fla., Jul. 29-Aug. 3, 1990.

Henner, J. H., Band, L. and Shimotsu, H. (1984) Nucleotide sequence of the Bacillus subtilis tryptophan operon. Gene, 34: 169-177.

Henner, J. H., Band, L., Flaggs, G. and Chen, E. (1986) The organization and nucleotide sequence of the Bacillus subtilis hisH, tyrA and aroE genes Gene 49: 147-152.

Hohn, B. and Collins J. (1980) A small cosmid for efficient cloning of large DNA fragments. Gene 11: 291-298.

Horsch, R. B. and H. Klee. (1986) Proc. Natl. Acad. Sci. U.S.A. 83:4428-32.

Hunkapiller. M. W., Hewick, R. M., Dreyer, R. J., and Hood, L. (1983) Methods Enzymol. 91, 399-413.

Jefferson, R. A., Kavanaugh, T. A. and Bevan, M. W., 1987, EMBO J., 6:3901-3907.

Kay, R., Chan, A., Daly, M. and McPherson, J. 1987. Duplication of the CaMV 35S promoter sequence creates a strong enhancer for plants. Science 236, 1299-1302.

Kishore, G., Shah, D., Padgette, S., della-Cioppa, G., Gasser, C., Re, D., Hironaka, C., Taylor, M., Wibbenmeyer, J., Eichholtz, D., Hayford, M., Hoffnan, N., Delannay, X, Horsch, R., KIee, H., Rogers, S., Rochester, D., Brundage, L., Sanders, P. and Fraley, R. T. (1988) 5-Enolpyruvylshikimate 3-phosphate synthase: From Biochemistry to genetic engineering of glyphosate tolerance, in Biotechnology for Crop Protection ACS Symposium series No. 379. Eds. Hedlin P. A., Menn, J. J. and Hollingsworth, R. M. pp. 3748.

Kishore, G. and Shah, D. (1988) Ann. Rev. Biochem. 57:627-663.

Kishore, G. M., Brundage, L., Kolk, K., Padgette, S. R., Rochester, D., Huynh, Q. K. and della-Cioppa, G. (1986) Fed. Proc. 45: 1506.

Klee, H. J., et al. (1985) Bio/Technolog 3:637-42.

Klee, H. J., Muskopf, Y. M. and Gasser, C. S. (1987) Cloning of an Arabidopsis thaliana gene encoding 5-enolpyruvyl-shilimate-3-phosphate synthase: sequence analysis and manipulation to obtain glyphosate-tolerant plants. Mol. Gen. Genet. 210: 437-442.

Klee, H. J. and Rogers, S. G. (1989) Plant gene vectors and genetic transformation: plant transformation systems based on the use of Agrobacterium tumefaciens in: Cell Culture and Somatic Cell: Genetics of Pants eds J. Schell and I. K. Vasil . 6: 1-23.

Klein, T. M. Kornstein. L. Sanford, J. C., and Fromm, M. E. 1989. Genetic transformation of maize cells by particle bombardment. Plant Phys. 91:440-444.

Koncz, C. and Schell, J. (1986) The promoter of TL-DNA gene 5 controls the tissue-specific expression of chimeric genes carried by a novel type of Agrobacterium binary vector. Mol. Gen. Genet. 204:383-396.

Kunkel, T. A, Roberts, J. D. and Zakour, R. A. (1987) Rapid and efficient site-specific mutagenesis without phenotypic selection. Methods Enzymol. 154:367.

Laemmli, U.K (1970), “Cleavage of structural proteins during the assembly of the head of the bacteriophage T4” Nature, 227:680.

Maliga, P., Carrer, H., Kanevski, I., Staub, J., and Svab, Z. (1993) Plastid engineering in land plants: a conservative genome is open to change. Philos. Trans. R. Soc. London B Biol. Sci. 342: 203-208.

Maniatis, T., Fritsch, E. F. and Sambrook, J. (1982) Molecular Cloning: a laboratory manual. Cold Spring Harbor Laboratory, Cold Spring Harbor, New York.

Maskell, D. J., Morrissey, P. and Dougan, G. (1988) Cloning and nucleotide sequence of the aroA gene of Bordetelia pertussis. J.Bacteriol. 170:2467-2471.

Miller, J. H. (1972). Experiments in Molecular Genetics. Cold Spring Harbor Laboratory, Cold Spring Harbor, New York.

Moore, J. K, Bravmer, H. D. and Larson. A. D. (1983) Isolation of a Pseudomonas sp. which utilizes the phosphonate herbicide glyphosate. Appl. Environ. Microbiol. 46: 316-320.

Morelli, G., Nagy, F., Fraley, R. T., Rogers, S. G., and Chua, N. H. (1985). A short conserved sequence is involyed in the light-inducibility of a gene encoding ribulose 1,5-bisphosphate carboxylase small subunit of pea. Nature 315, 200-204.

O'Connell, C., Pattee, P. A. and Foster, T. J. (1993) Sequence and mapping of the aroA gene of Staphylococcus aureus 83254. J. Gen. Micr. 139: 1449-1460.

Odell, J. T., Nagy, F., and Chua, N.H. (1985). Identification of DNA sequences required for activity of the cauliflower mosaic virus 35S promoter. Nature 313, 810-812.

Olins, P. O., Devine, C. S., Rangwala, S. H. and Kavka, K. S. (1988) Gene 73: 227-235.

O'Neill, C., Horvath, G. V., Horvath, E., Dix, P. J. and Medgyesy, P. (1993) Chloroplast transformation in plants: polyethylene glycol (PEG) treatment of protoplasts is an alternative to biolistic delivery systems. Plant J. 3: 729-738.

Padgette, S. R., Huynh, Q. K, Borgmeyer, J., Shah, D. M., Brand, L. A., Re, D. B., Bishop, B. F., Rogers. S. G., Fraley, R. T., and Kishore, G. (1987) Bacterial expression and isolation of Petunia hybrida 5enol-pyruvylshikimate-3-phosphate synthase. Arch. Biochem. Bionhys. 258, 564-573.

Padgette, S. R., Huynh. Q. K. Aykent, S., Sammons, R. D., Sikorski, J. A, and Kishore, G. M. (1988) J. Biol. Chem. 263, 1798-1802.

Petersen, W.L., Sulc, S., and Armstrong, C. L. 1992. Effect of nurse cultures on the production of macro-calli and fertile plants from maize embryogenic suspension protoplasts. Plant Cell Reports 10:591-594.

Quinn, J. P., Peden, J. M. M. and Dick, E. (1988) Glyphosate tolerance and utilization by the microflora of soils treated with the herbicide. Appl. Microbiol. Biotechnol. 29: 511-516.

Rao, R. N. and Rogers, S. G. (1979). Plasmid pKC7: A vector containing ten restriction endonuclease sites suitable for cloning DNA segments. Gene 7:79.

Richins, R. D., Scholthof, H. B., and Shepard, R. J. (1987) Sequence of the figwort mosaic virus DNA (caulimovirus group). Nucl. Acids Res. 15: 8451-8466.

Rogers, S. G., Brand, L. A. Holder, S. B. Sharps, E. S. and Brackin, M. J. (1983) Amplification of the aroA gene from E. coli results in tolerance to the herbicide glyphosate. Appl. Environ. Microbiol. 46:37-43.

Rogers, S. G. and Klee, H. J. (1987). “Pathways to genetic manipulation employing Agrobacterium.” in Plant Gene Research, Plant DNA Infectious Agents, Vol IV, Hohn, T. and Schell, J., eds. Springer-Verlag, Vienna, pp. 179-203.

Sambrook, J., Fritsch, E. F. and Maniatis, T., (1989) Molecular Cloning A Laboratory Manual, Cold Spring Harbor Laboratory Press, Cold Spring Harbor. New York.

Schocher, R. J., Shillito, R. D., Saul, M. W., Paszkowski, J., and Potrykus, l. (1986). Co-transformation of unlinked foreign genes into plants by direct gene transfer. Bio/Technoloyg 4:1093-1097.

Songstad, D. D., Armstrong, C. L., and Petersen, W. L. (1991). AgNO₃ increases type II callus production from immature embryos of maize inbred B73 and its derivatives. Plant Cell Reports 9: 699-702.

Schuler, M. A, Schmitt, E. S. and Beachy, R. N. (1982) Nucleic Acids Res. 10:8225-8244.

Schulz, A., Kruper, A. and Amrhein, N. (1985) Differential sensitivity of bacterial 5-enolpyruvylshikimate-3-phosphate synthases to the herbicide glyphosate. FEMS Microbiol. Lett. 28: 297-301.

Schulz, A., Sost, D. and Amrhein, D. (1984) Arch. Microbiol. 137: 121-123.

Shah, D., Horsch, R., KIee, H., Kishore, G., Winter, J., Tumer, N., Hironaka, C., Sanders, P., Gasser, C., Aykent, S. Siegal, N., Rogers, S., and Fraley, R. (1986). Engineering herbicide tolerance in transgenic plants. Science 233, 478-481.

Shah, D. M., Rochester, D. E., Krivi, G., Hironaka, C., Mozer, T. J., Fraley, R. T., and D. C. Tiemeier. 1985. Structure and expression of the maize hsp70gene. Cell. and Mol. Biol. of Plant Stress. Alan R. Liss, Inc. pp. 181-200.

Shimamoto, K. et al. (1989) Nature 338:274-276.

Sost, D., Schulz. A. and Amrhein, N (1984) FEBS Lett. 173: 238-241.

Sost, D. and Amrhein, N. (1990) Substitution of Gly-96 to Ala in the 5-enolpyruvylshikimate 3-phosphate synthase of Klebsiella pneumoniae results in greatly reduced affty for the herbicide glyphosate. Arch. Biochem. Biophys. 282: 433-436.

Stalker, D. M., Thomas, C. M., and Helinski, D.R. (1981). Nudeotide sequence of the region of the origin of replication of the broad host range plasmid RK2. Mol Gen Genet 181: 8-12.

Stalker, D. M., Hiatt, W. R. and Comai, L. (1985) A single amino acid substitution in the enzyme 5-enolpyruvylshikimate 3-phosphate synthase confers resistance to glyphosate. J. Biol. Chem. 260: 4724-4728.

Stallings, W. C., Abdel-Meguid, S. S., Lim, L. W., Shieh, Huey-Sheng, Dayringer, H. E., Leimgruber, N. K., Stegeman, R. A., Anderson, K. S., Sikorski, J. A., Padgette S. R., Kishore, G. M. (1991). Structure and Topological Symmetry of the Glyphosate Target 5-enol-pyruvylshikindate-³-phosphate synthase. Proc. Natl. Acad. Sci. USA 88. 5046-5050.

Svab, Z., Hajdukiewicz, P., and Maliga, P. (1990) Stable transformation of plastids in higher plants. Proc. Natl. Acad. Sci. USA 87: 8526-8530.

Svab, Z. and Maliga, P. (1993) High frequency plastid transformation in tobacco by selection for a chimeric aadA gene. Proc. Natl. Acad Sci. USA 90:913-917.

Tabor, S. and Richardson. C. C. (1985) A bacteriophage T7 RNA polymerase/promoter system for controlled exclusive expression of specific genes. Proc. Natl. Acad. Sci. USA 82: 1074-1078.

Talbot, H. W., Johnson, L. M. and Munnecke, D. M. (1984) Glyphosate utilization by Pseudomonas sp. and Alcaligenes sp. isolated from environmental sources. Current Microbiol. 10: 255-260.

Talmadge, K, and Gilbert, W., (1980) “Construction of plasmid vectors with unique PstI cloning sites in the signal sequence coding region” Gene, 12: 235-241.

Timko, M. P., Herdies, L., de Almeida, E., Cashmore, A.R., Leemans, J., and Krebbers, E. 1988. Genetic Engineering of Nuclear-Encoded Components of the Photosynthetic Apparatus in Arabidopsis in “The Impact of Chemistry on Biotechnology,” ACS Books, 279-295.

Vasil, V., F. Redway and I. Vasil. (1990), Bio/Technology 8:429434.

Vieira, J. and Messing J. (1987) Production of single-stranded plasmid DNA. Methods Enzvmol. 153: 3-11.

Yanisch-Perron, C., Vieira, J. and Messing, J. (1985). Improved M13 phage cloning vectors and host strains: nucleotide sequences of the M13mp18 and pUC19 vectors. Gene 33, 103-119

1. An isolaed DNA sequence other than the structural coding sequence listed in SEQ ID NO:41, SEQ ID NO:43 SEQ ID NO:66 and SEQ ID NO:68, encoding an EPSPS enzyme having the sequence domains:
—R—X₁—H—X₂—E—(SEQ ID 50:37), in which
X₁ is G, S, T, C, Y, N, Q, D or E;
X₂ is S or T; and
—GD—K—X₃—(SEQ ID NO:38), in which
X₃ is S or T; and
—S—A—Q—X₄—K—(SEQ ID NO:39), in which
X₄is A, R, N, D, C, Q, E, G, H, I, L, K, M, F, P, S, T, W, Y or V; and
—N—X,—T—R—(SEQ ID NO:40), in which
X₆ is A, R, N, D, C, Q, E, G, I, L, K, M, F, P, S, T, W, Y or V.

2. A DNA molecule of claim 1 in which the Kₘ forphosphoenolpyruvate is between 2 and 25 μM.

3. A DNA molecule of claim 1 in which the K₁/Kₘ ratio is between 25 and 500.

4. A DNA molecule of claim 1 in which X₁ is D or N; X₂ is S or T; X₃ is S or T; X₄is V, I or L; and X₅ is P or Q.

5. A DNA molecule of claim 4 which encodes an EPSPS enzyme having the sequence of SEQ ID NO:3.

6. A DNA molecule of claim 5 having the sequence of SEQ ID NO:2.

7. A DNA molecule of claim 5 having the sequence of SEQ IDNO:9.

8. A recombinant, double-stranded DNA molecule comprising in sequence:
a) a promoter which functions in plant cells to cause the production of an RNA sequence;
b) a structural DNt sequence that causes the production of an RNA sequence which encodes a EPSPS enzyme having the sequence domains:
—R—X₁—H—X₂—E—(SEQ ID NO:37), in which
X₁ is G, S, T, C, Y, N, Q, D or E;
X₂ is S or T; and
—GD—K—X₃—(SEQ ID NE :386 which
X₃ is S or T; and
—S—A—Q—X₄—K—(SEQ ID NO:39), in which
X₄is A, R, N, D, C, Q, E, G, H, I, L, K, M, F, P, S, T, W, Y or V; and
—N—X₅—T—R—(SEQ ID NO:40), in which
X₅is A, R, N, D, C, Q, E, G, H, I, L, K, M, F, P, S, T, W, Y or V; and
c) a 3′ non-translated region which functions in plant cells to cause the addition of a stretch of polyadenyl nucleotides to the 3′ end of the RNA sequence;
where the promoter is heterologous with respect to the structural DNA sequence and adapted to cause sufficient expression of the encoded EPSPS enzyme to enhance the glyphosate tolerance of a plant cell transformed with the DNA molecule.

9. A DNA molecule of claim 8 in which the structural DNA sequence encodes a fusion polypeptide comprising an amino-terminal chloroplast transit pep de and the EPSPS enzyme.

10. A DNA molecule of claim 8 in which X₁ is D or N; X₂ is S or T; X₃ is S or T; X₄is V, I or L; and X₅ is P or Q.

11. A DNA molecule of claim 10 in which the ftructural DNA sequence encodes an EPSPS enzyme selected from the group consisting of SEQ ID NO:2, SEQ ID NO:4, SEQ ID NO:6, SEQ ID NO:41 and SEQ ID NO:43.

12. A DNA molecule of claim 9 in which X₁ is D or N; X₂ is S or T; X₃ is S or T; X₄ is V, I or L; and X₅ is P or Q.

13. A DNA molecule, of claim 12 in which the structural DNA sequence encodes an EPSPS enzyme selected from the group consisting of SEQ ID NO:2. SEQ ID NO:4, SEQ ID NO:6, SEQ ID NO:41 and SEQ ID NO:43.

14. A DNA molecule of claim 12 in which the EPSPS sequence is SEQ ID NO:3.

15. A DNA molecule of claim 14 in which the promoter is a plant DNA virus promoter.

16. A DNA molecule of claim 15 in which the promoter is selected from the group consisting of CaMV35S and FMV35S promoters.

17. A DNA molecule of claim 14 in which the the structural DNA sequence encodes a chioroplast transit peptide selected from the group consisting of SEQ ID 0:11 and SEQ ID NO:15.

18. A DNA molecule of claim 17 in which the 3′ non-translated region is selected from the group consisting of the NOS 3′ and the E9 3′ non-translated regions.

19. A method of producing genetically transformed plants which are tolerant toward glyphosate herbicide, comprising the steps of:
a) inserting into the genome of a plant cell a recombinant, double-stranded DNA molecule comprising:
i) a promoter which functions in plant cells to cause the production of an RNA sequence,
ii) a structural DNA sequence that causes the production of an RNA sequence which encodes an EPSPS enzyme having the sequence domains:
—R—X₁—H—X₂—E—(SEQ ID NO:37), in which
X₁ is G, S, T, C, Y, N, Q, D or E;
X₂ is S or T; and
—G—D—K—X₃—(SEQ ID NO:38), in which
X₃ is S or T; and
—S—A—Q—X₄—K—(SEQ ID NO:39), in which
X₄ is A, R, N, D, C, Q, E, G, H, I, L, K, M, F, P, S, T, W, Y or V; and
—N—X₅—T—R—(SEQ ID NO:40), in which
X is A, R, N, D, C, Q, E, G, H, L, K, M, F. P, S, T, W, Y or V; and
iii) a 3′ non-translated DNA sequence which functions in plant cells to cause the addition of a stretch of polyadenyl nucleotides to the 3′ end of the RNA sequence;
where the promoter is herologous with respect to the structural DNA sequence and adapted to cause sufficient expression of the polypeptide to enhance the glphosate tol rance of a plant cell transformed with the DNA molecule;
b) obtaining a transformed plant cell; and
c) regenerating frog the transformed plant cell a genetically transformed plant which has increased tolerance to glyphosate herbicide.

20. A method of claim 17 in which X₁ is D or N; X₂ is S or T; X₃ is S or T; X₄ is V, I or L; and X₅ is P or Q.

21. A method of claim 20 in which the structural DNA sequence encodes an EPSPS enzyme selected from the group consisting of SEQ ID NO:2, SEQ ID NO:4, SEQ ID NO:6, SEQ ID NO:41 and SEQ ID NO:43.

22. A method of claim 19 in which the structural DNA sequence encodes a fusion polypeptide comprising an amino-terminal chloroplast transit peptide and the EPSPS enzyme.

23. A method of claim 22 in which X₁ is D or N; X₂ is S or T; X₃ is S or T; X₄ is V, I or L; and X₅ is P or Q.

24. A method of claim 23 in which the structural DNA sequence encodes an EPSPS enzyme selected from the group consisting of SEQ ID NO:2, SEQ ID NO:4, SEQ ID NO:6, SEQ ID NO:42 and SEQ ID NO:44.

25. A method of claim 23 in which the EPSPS enzyme is that set forth in SEQ ID NO:3.

26. A method of claim 25 in which the promoter is from a plant DNA virus.

27. A method of claim 26 in which the promoter is selected from the group consisting of CaMV35S and FE W35S promoters.

28. A glyphosate-tolerant plant cell comprising a DNA molecule of claims 9, 12 or 14.

29. A glyphosate-tolerant plant cell of claim 28 in which the promoter is a plant DNA virus promoter.

30. A glyphosate-tolerant plant cell of claim 29 in which the promoter is selected from the group consisting of CaMV35S and FMV35S promoters.

31. A glyphosate-tolerant plant cell of claim 28 selected from the group consisting of corn, wheat, rice, barley, soybean, cotton, sugarbeet, oilseed rape, canola, flax, sunflower, potato, tobacco, tomato, alfalfa, poplar, pine, eukalyptus, apple. lettuce, peas, lentils, grape and turf grasses.

32. A glyphosate-tolerant plant comprising plant cells of claim 31.

33. A glyphosate-tolerant plant of claim 32 in which the promoter is from a DNA plant virus promoter.

34. A glyphosate-tolerant plant of claim 33 in which the promoter is selected from the group csisting of CaMV35S and FMV35S promoters.

35. A glyphosate-tolerant plant of claim 34 selected from the group consisting of corn, wheat, rice, barley, soybean, cotton, sugarbeet, oilseed rape, canola, flax, sunflower, potato, tobacco, tomato, alfalfa, poplar, pine, eukalyptus, apple, lettuce, peas, lentils, grape and turf grasses.

36. A method for selectively controlling weeds in a field containing a crop having planted crop seeds or plants comprising the steps of:
a) planting the crop seeds or plants which are glyphosate-tolerant as a result of a recombinant doubstranded DNA molecule being inserted into the crop seed or plant, the DNt molecule having:
i) a promoter whic ftntions in plant cells to cause the production of an RNA sequence,
ii) a structural DNA sequence that causes the production of an RNA sequence which encodes an EPSPS enzyme having the sequence domains:
—R—X₁—H—X₂—E—(SEQ ID NO:37), in which
X₁ is G, S. T, C, Y, N, Q, D or E;
X₂ is S or T; and
—GD—K—X₃—(SEQ ID NO:38), in which
X is S or T; and
—S—A—Q—X₄—K—(SEQ ID NO:39), in which
X₄ is A, R, N, D, C, Q, E, G, H, I, L, K, M, F, P, S, T, W, Y or V; and
—N—X₅—T—R—(SEQ ID NO:40), in which
X₅ is A, R, N, D, C, Q, E, G, H, L, L, K, F, P, S, T, W, Y or V; and
iii) a 3′ non-translated DNA sequence which functions in plant cells to cause the addition of a stretch of polyadenyl nucleotides to the 3′ end of the RNA sequence
where the promoter is eterologous with respect to the structural DNA sequence and adapted to cause sufficient expression of the EPSPS enzyme to enhance the glyphosate tolerance of the crop plant transformed with the DNA molecule; and
b) applying to the crop and weeds in the field a sufficient amount of glyphosate herbicide to control the weeds without significantly affecting the crop.

37. A method of claim 36 in which X₁ is D or N; X₂ is S or T; X₃ is or T; X₄ is V, I or L; and X₅ is P or Q.

38. A method of in which the structural DNA sequence encodes an EPSPS enzyme selected from the sequences as set forth in SEQ ID NO:2, SEQ ID NO:4, SEQ ID NO:6, SEQ ID NO:42 and SEQ ID NO:44.

39. A method of claim 36 in which the structural DNA sequence encodes a fusion polypeptide comprising an amino-terminal chioroplast transit peptide and the EPSPS enzyme.

40. A method of claim 39 in which X₁ is D or N; X₂ is S or T; X₃ is S or T; X₄ is V, I or L; and X is P or Q.

41. A method of claim 40 in which the structural DNA sequence encodes an EPSPS enzyme selected from the sequences as set forth in SEQ ID NO:2, SEQ ID NO:4, SEQ ID NO:6, SEQ ID,NO:41 and SEQ ID NO:43.

42. A method of claim 40 in which the DNA molecule encodes an EPSPS enzyme as set forth in SEQ ID NO:3.

43. A method of claim 42 in which the DNA molecule further comprises a promoter selected from the group consisting of the CAMV35S and FMV35S promoters.

44. A method of claim 43 in which the crop plant is selected from the group consisting of corn, wheat, rice, barley, soybean, cotton, sugarbeet, oilseed rape, canola, flax, sunflower, potato, tobacco, tomato, alfalfa, poplar, pine, eukalyptus, apple, lettuce, peas, lils, grape and turf grasses.

45. A DNA molecule of claim 9 in which the structural DNA sequence encodes a chloroplast itfansit peptide selected from the group consisting of SEQ ID NO:11, SE9 ID NO:13, SEQ ID NO:15 and SEQ ID NO:17.

46. A DNA molecule of claim 45 in which the chloroplast transit peptide is encoded by a DNA sequence selected from the group consisting of SEQ ID NO:10, SEQ ID NO:12, SEQ ID NO:14 and SEQ ID NO:16.

47. A DNA molecule of claim 9 in which the structural DNA sequence encodes a chloroplast transit peptide selected from the group consisting of SEQ ID NO:11 and SEQ ID NO:15.

48. A DNA molecule of claim 47 in which the chloroplast transit peptide is encoded by a DNA sequence selected from the group consisting of SEQ ID NO:10 and SEQ ID NO:14.

49. A DNA molecule of claim 45 in which the promoter is selected from the group consisting o;M 35S and FMV 35S promoters.

50. A DNA molecue of claim 46 in which the promoter is selected from the group consisting of daMV35S and FMV 355 promoters.

51. A DNA molecule of claim 47 in which the promoter is seledted from the group consisting of CAMV 35S and FMV 35S promoters.

52. A DNA molecule of claim 48 in which the promoter is selected from the group consisting of CAMV 3S and FMV 35S promoters.

53. A DNA molecule of Cla 49 in which the 3′ non-translated region is selected from the group consistig of the NOS 3′ and the E9 3′ non-translated regions.

54. A DNA molecule of claim 50 in which the 3′ non-translated region is selected from the group consisting of the NOS 3′ and the E9 3′ non-translated regions.

55. A DNA molecule of claim 51 in which the 3′ non-translated region is selected from the group consisting of the NOS 3′ and the E9 3′ non-translated regions.

56. A DNA molecule of claim 52 in which the 3′ non-translated region is selected from the group consisting of the NOS 3′ and the E9 3′ non-translated regions.

57. A DNA molecule of claim 53 in which the structural DNA sequence encodes an EPSPS enzyme selected from the group consisting of SEQ ID NO:3, SEQ ID NO:5, SEQ ID NO:7, SEQ ID NO:42 and SSEQ ID NO:44.

58. A DNA molecule of claim 54 in which the structural DNA sequence encodes an EPSPS enzyme selected from the group consisting of SEQ ID NO:3, SEQ ID NO:5, SEQ ID NO:7, SEQ ID NO:42 and SEQ ID NO:44.

59. A DNA molecule of claim 55 in which the structural DNA sequence encodes an EPSPS enzyme selected from the group consisting of SEQ ID NO:3, SEQ ID NO:5, SEQ ID NO:7, SEQ ID NO:42 and SEQ ID NO:44.

60. A DNA molecule of claim 56 in which the structural DNA sequence encodes an EPSPS enzyme selected from the group consisting of SEQ ID NO:3, SEQ ID NO:5, SEQ ID NO:7, SEQ ID NO:42 and SEQ ID NO:44.

61. A DNA molecule of claim 57 in which the structural DNA sequence contains an EPSPS encoding sequence selected from the group consisting of SEQ ID NO:2, SEQ ID NO:4, SEQ ID NO:6, SEQ ID NO:41 and SEQ ID NO:43.

62. A DNA molecule of claim 58 in which the structural DNA sequence contains an EPSPS encoding sequence selected from the group consisting of SEQ ID NO:2, SEQ ID NO:4, SEQ ID NO:6, SEQ ID NO:41 and SEQ ID NO:43.

63. A DNA molecule of claim 59 in which the structural DNA sequence contains an EPSPS encoding sequence selected from the group consisting of SEQ ID NO:2, EQ ID NO:4, SEQ ID NO:6, SEQ ID NOA1 and SEQ ID NO:43.

64. A DNA molecue of claim 60 in which the structural DNA sequence contains an EPSPS encoding sequence selected from the group consisting of SEQ ID NO:2, SEQ ID NO4, SEQ ID NO:6, SEQ ID NO:41 and SEQ ID NO:43.

65. A DNA molecule of claim 53 in which the structural DNA sequence encodes an EPSPS enzyme having the sequence of SEQ ID NO:3.

66. A DNA molecule am 54 in which the structural DNA sequence encodes an EPSPS enzyme the sequence of SEQ ID NO:3.

67. A DNA molecule of claim 55 in which the structural DNA sequence encodes an EPSPS enzyme having the sequence of SEQ ID NO:3.

68. A DNA molecule of claim 56 in which the structural DNA sequence encodes an EPSPS enzyme having the sequence of SEQ ID NO:3.

69. A DNA molecule of claim 65 in which the structural DNA sequence contains an EPSPS encoding sequence selected from the group consisting of SEQ ID NO:2 and SEQ ID NO:9.

70. A DNA molecule of claim 66 in which the structural DNA sequence contains an EPSPS encoding sequence selected from the group consisting of SEQ ID NO:2 and SEQ ID NO:9.

71. A DNA molecule of claim 67 in which the structural DNA sequence contains an EPSPS encoding sequence selected from the group consisting of SEQ ID NO:2 ad SEQ ID NO:9.

72. A DNA molecule of claim 68 in which the structural DNA sequence contains an EPSPS encoding sequence selected from the group consisting of SEQ ID NO:2 and SEQ ID NO:9.

73. A glyphosate-tolerant plant cell of claim 29 in which:
(a) the promoter is selected from the group consisting of CaMV 35S and FMV 35S promoters;
(b) the structural DNA sequence encodes
(i) a chloroplast,/Cansit peptide selected from the group consisting of SEQ ID NO 11, SEQ ID NO:13, SEQ ID NO:15 and SEQ ID NO:17;
(ii) an EPSPS selected from the group consisting of SEQ ID NO:3, SEQ ID NO:5; SEQ ID NO:7, SEQ ID NO:42 and SEQ ID NO:44; and
(c) the 3′ non-translated region is selected from the group consisting of the NOS 3′ and the E9 3′ non-translated regions.

75. A glyphosate-tolerant plant cell of claim 73 in which the structural DNA sequence comprises:
(a) a chloroplast transit peptide encoding DNA sequence selected from the group consisting of SEQ ID NO:10, SEQ ID NO:12, SEQ ID NO:14 and SEQ ID NO:16; and
(b) an EPSPS encoding sequence selected from the group consisting of SEQ ID NO:2, SEQ ID NO:4, SEQ ID NO:6, SEQ ID NO:41 and SEQ ID NO:43.

76. A glyphosatl-tolerant plant cell of claim 73 in which the structural DNA sequence comprises:
(a) a chloroplast ransit peptide encoding DNA sequence selected from the group consisting of EQ ID NO:10 and SEQ ID NO:14; and
(b) a DNA sequence encoding an EPSPS enzyme having the sequence of SEQ ID NO:3.

77. A glyphosate-tolerant plant cell of claim 74 in which the structural DNA sequence comprises an EPSPS encoding sequence selected from the group consisting of SEQ ID NO:2 and SEQ ID NO:9.

78. A glyphosate-tolerant plant cell of claim 75 selected from the group consisting of corn, wheat rice barley, soybean, cotton, sugarbeet, oilseed rape, canola, flax, sunflower, tobacco, tomato, alfalfa, poplar, pine, eukalyptus, apple, lettuce, peas, lentl₇i grape and turf grasses.

79. A glyphosate-tolerant plant comprising a DNA molecule of claims 9, 12 or 14 in which:
(a) the promoter is selected from the group consisting of CaMV 35S and FMV 35S promoters;
(b) the structural DNA sequence encodes:
(i) a chloroplast transit peptide selected from the group consisting of SEQ ID NO:11, SEQ ID NO:13, SEQ ID NO:15 and SEQ ID NO:17; and
(ii) an EPSPS enzyme selected from the group consisting of SEQ ID NO:3, SEQ ID NO:5, SEQ ID NO:7, SEQ ID NO:42 and SEQ ID NO:44: and
(c) the 3′ non-translated region is selected from the group consisting of the NOS 3′ and the E9 3′ non-translated regions.

80. A glyphosate-tolerant plant of claim 79 in which the structural DNA sequence comprises:
(a) a chioroplast transit peptide encoding DNA sequence selected from the group consisting of SEQ ID NO:10, SEQ ID NO:12, SEQ ID NO:14 and SEQ ID NO:16; and
(b) an EPSPS encoding sequence selected from the group consisting of SEQ ID NO:2, SEQ ID NOA4, SEQ ID NO:6, SEQ ID NO:41 and SEQ ID NO:43.

81. A glyphosate-tolerantjlant of claim 80 in which the structural DNA sequence comprises:
(a) a chloroplast tran⁴i peptide encoding DNA sequence selected from the group consisting of SEQ/ID NO:10 and SEQ ID NO:14; and
(b) a DNA sequence encoding an EPSPS enzyme having the sequence of SEQ ID NO:3.

82. A glyphosate-tolarant plant of claim 81 in which the structural DNA sequence comprises an EPSPS encoding sequence selected from the group consisting of SEQ ID NO:2 and SEQID NO:9.

83. A glyphosate-tolerant plant of claim 82 selected from the group consisting of corn, wheat, rice, barley, soybean, cotton, sugarbeet, oilseed rape, canola, flax, sunflower, potato, tobacco, tomato. alfalfa, poplar, pine, eukalyptus, apple, lettuce, peas, lentils, grape and turf grasses.

84. A seed of a glyphosate-tolerant plant of claim 32.

85. A seed of a glyphosate-tolerant plant of claim 35.

86. A seed o glyphosate-tolerant plant of claim 79.

87. A seed of a glyphosate-tolerant plant of claim 80.

88. A seed of a glyphosate-tolerant plant of claim 81.

89. A seed of a glyphosate-tolerant plant of claim 82.

90. A seed of a glyphosate-tolerant plant of claim 83.

91. A transgenic soybean plant which contains a heterologous gene which encodes an EPSPS enzyme having a Kₘ for phosphoenolpyruvate (PEP) between 1 and 150 μM and a Kₘ (glyphosate)/Km(PEP) ratio between about 2 and 500, said plant exhibiting tolerance to N-phosphonomethylglycine herbicide at a rate of 1 lb/acre without significant yield reduction due to herbicide application.

92. Seed of a soybean plant of claim 91.

93. In a method for the transformation and regeneration of transgenic plants, the improvement which comprises the use of a glyphosate-resistance marker gene comprising:
i) a promoter which functions in plant cells to cause the production of an RNA sequence,
ii) a structural DNA sequence that causes the production of an RNA sequence which encodes an EPSPS enzyme having the sequence domains:
—R—X₁—H—X₂—E—(SEQ ID NO:37), in which
X₁ is G, S, T, C, Y, N, Q, D or E;
X₂ is S or T; and
—GD—K—X₃—(SEQ ID NO:38), in which
X₃is S or T; and
—S—A—Q—X₄—K—(SEQ ID NO:39), in which
X₄is A, R, N, D, C, Q, E, G, H, I, L, M, F, P, S, T, W, Y or V; and
—N—X₅—T—R—(SEQ ID NO:40), in which
X₅ is A, R, N, D, C, Q, E, G, H, I, L, K, M, F, P, S, T, W, Y or V; and
iii) a 3′ non-translated DNA sequence which functions in plant cells to cause the addition of a stretch of polyadenyl nucleotides to the 3′ end of the RNA sequence;
where the promoter is heterologous with respect to the structural DNA sequence and adapted to cause sufficient expression of the polypeptide to render a plant cell transformed witW the DNA molecule tolerance to a toxic level of glyphosate.

94. A method of claim 93 in which X₁ is D or N; X₂ is S or T; X₃ is S or T; X₄ is V, I or L; and X₅ is P or Q,

95. A method of claim 94 in which the structural DNA sequence encodes an EPSPS enzyme selected from the group consisting of SEQ ID NO:2, SEQ ID NO:4, SEQ ID NO:6, SEQ ID NO:41 and SEQ ID NO:43.

96. A method of claim 93 in which the structural DNA sequence encodes a fusion polypeptide comprising an amino-terminal chloroplast transit peptide and the EPSPS enzyme.

97. A method of claim 96 in which X₁ is D or N; X₂ is S or T; X₃ is S or T; X₄ is V, I or L; and X₅ is P or Q.

98. A method of claim 97 in which the structural DNA sequence encodes an EPSPS enzyme selected from the group consisting of SEQ ID NO:2, SEQ ID NO:4, SEQ ID NO:0:6, SEQ ID NO:42 and SEQ ID NO:44.

99. A method of claim 93 in which the EPSPS enzyme is that set forth in SEQ ID NO:3.

100. A method of claim 99 in which the promoter is selected from the group consisting of CaMV35S and F 3MV53S promoters.
